(19) United States
(12) Patent Application Publication
(10) Pub. No.: US 2023/0122540 A1
(43) Pub. Date: Apr. 20, 2023

(54) SYSTEMS AND METHODS TO TRACK THE EVOLUTION OF SINGLE CELLS

(71) Applicant: Genentech, Inc., South San Francisco, CA (US)

(72) Inventors: Xin YE, South San Francisco, CA (US); Matthew Tsn-Wei CHANG, South San Francisco, CA (US)

(21) Appl. No.: 17/932,885

(22) Filed: Sep. 16, 2022

Related U.S. Application Data
(63) Continuation of application No. PCT/US2021/023274, filed on Mar. 19, 2021.
(60) Provisional application No. 63/161,390, filed on Mar. 15, 2021.
(60) Provisional application No. 62/992,498, filed on Mar. 20, 2020.

(51) Int. Cl. C12N 15/10; C12N 5/09; C12Q 1/6869

(52) U.S. Cl. CPC C12N 15/1065; C12N 5/0693; C12Q 1/6869; C12N 2503/02

(57) ABSTRACT

Cells in a given population often display heterogeneity that may affect how each cell responds to a particular treatment or growth condition. The methods described herein allow determination of which cells from an initial population survive a treatment or condition, and how surviving cells evolve over time. For example, the methods described herein may be used to model drug resistance, response and/or adaptation in a cell population.

### CROSS-REFERENCE TO RELATED APPLICATIONS

This application is a continuation of International Application No. PCT/US2021/023274, filed Mar. 19, 2021, which claims priority to U.S. provisional application No. 63/161,390 filed on Mar. 15, 2021, and 62/992,498 filed Mar. 20, 2020, each of which are incorporated herein by reference in their entireties.

### BACKGROUND

Although there have been remarkable advances in cancer therapy in recent years, tumor cells often develop resistance to therapies that allow them to escape death. These resistant cells then seed relapsed tumors that require a secondary treatment, if one is available. Co-treatment with multiple therapies have been clinically shown to provide additional benefits to patients, partly by overcoming resistance. It is desirable to understand the mechanisms of resistance to various treatments in order to develop more efficient treatments and treatment combinations for cancer.

### SUMMARY

Cells in a given population often display heterogeneity that may affect how each cell responds to a particular treatment or growth condition. The methods described herein allow determination of which cells from an initial population survive a treatment or condition, and how such cells survive the treatment and/or evolve over time. For example, the methods described herein may be used to model drug resistance, response and/or adaptation in a cell population.

The instant technology generally allows tracking of single cell clones over time and comparison of their fitness under a selective pressure of interest. The fitness of each single cell clone can be determined by the relative size of that clone over time, which may be measured for example by the abundance of a unique barcode that was genetically introduced into that clone. At the same time, each single cell clone can be analyzed, e.g. by single cell RNA-sequencing (scRNA-seq) to obtain transcription status at single cell resolution before, during and after the selection. The transcription program associated with “winner” clones (cells with enriched barcodes) can be compared to the “loser” clones (cells with depleted barcodes) to identify pre-existing traits as well as adaptive changes that enable survival under a selective pressure of interest, such as but not limited to drug treatment, genomic engineering, and engraftment into a host.

In an aspect, a method for screening cells for a trait is provided. The method may include: (a) obtaining a plurality of barcoded cells, wherein each barcoded cell includes a single, unique barcode, or barcode combinations; (b) performing a first sequencing of RNA and/or DNA on a subset of the plurality of barcoded cells; (c) culturing the plurality of barcoded cells in the presence of a selection pressure for a first period of time, thereby forming a first plurality of cells; (d) performing a second sequencing of RNA and/or DNA on a subset of the first plurality of cells; (e) culturing the first plurality of cells in the presence of the selection pressure for a second period of time, thereby forming a second plurality of cells; and (f) performing a third sequencing of RNA and/or DNA on at least a subset of the second plurality of cells. The method may also include: (g) determining the relative abundance of a barcode sequenced in the first sequencing, second sequencing, and/or third sequencing.

In another aspect, a method for screening cells for a response trait to a therapeutic agent is provided. The method may include: (a) obtaining a plurality of barcoded cells, wherein each barcoded cell comprises a single, unique barcode; (b) performing a first sequencing of RNA and/or DNA on a subset of the plurality of barcoded cells; (c) culturing the plurality of barcoded cells in the presence of the therapeutic agent for a first period of time, thereby forming a first plurality of cells; (d) performing a second sequencing of RNA and/or DNA on a subset of the first plurality of cells; (e) culturing the first plurality of cells with the therapeutic agent for a second period of time, thereby forming a second plurality of cells; and (f) performing a third sequencing of RNA and/or DNA on a subset of the second plurality of cells. The method may also include: (g) determining the relative abundance of a barcode sequenced in the first sequencing, second sequencing, and/or third sequencing.

In another aspect is provided a method for comparing responses to selective pressures. The method may include: (a) obtaining a first plurality of barcoded cells, wherein each barcoded cell comprises a single, unique barcode; (b) obtaining a second plurality of barcoded cells that is substantially similar to the first plurality of barcoded cells; (c) performing a first sequencing of RNA and/or DNA from the first plurality of barcoded cells and/or the second plurality of barcoded cells; (d) culturing the first plurality of barcoded cells in the presence of a first selection pressure, thereby forming a first plurality of cells; (e) culturing the second plurality of barcoded cells in the presence of a second selection pressure, thereby forming a second plurality of cells; (f) performing a second sequencing of RNA and/or DNA from the first plurality of cells and/or the second plurality of cells; (g) culturing the first plurality of cells in the presence of the first selection pressure, thereby forming a third plurality of cells; (h) culturing the second plurality of cells in the presence of the second selection pressure, thereby forming a fourth plurality of cells; and (i) performing a third sequencing of RNA and/or DNA from the third plurality of cells and/or the fourth plurality of cells. The method may also include: (j) determining the relative abundance of one or more barcodes sequenced in the first sequencing, second sequencing, and/or third sequencing.

In an aspect is provided method of screening cells for a trait in a cell. The method may include: (a) providing a mixture of cells comprising multiple clonal populations wherein each clonal population comprises an identifier that is unique to the respective clonal populations, and wherein initial genetic, transcriptomic, and/or proteomic information of at least one representative member of each clonal population is known; (b) culturing the mixture of cells in the presence of a first selective pressure for a first period of time, and at the end of the first period of time, obtaining second genetic, transcriptomic, and/or proteomic information for at least one member of a surviving clonal population from within the mixture of cells; (c) subjecting the mixture of cells that were subjected to the first selective pressure to a second selective pressure for a second period of time, and at the end of the second period of time, obtaining third genetic, transcriptomic, and/or proteomic information of at least one member of a surviving clonal population from within the mixture of cells; and (d) determining the relative abundance of each clonal population present in the final mixture of cells based upon the unique identifier for the clonal population. The method may also include (e) identifying an adaptive trait, wherein the adaptive trait is a genetic and/or proteomic trait present in or absent from a clonal population in the final mixture of cells. The method may also include (f) comparing information from the initial genetic, transcriptomic, and/or proteomic information, second genetic, transcriptomic, and/or proteomic information, and/or third genetic, transcriptomic, and/or proteomic information, and/or one or more subsequent genetic, transcriptomic, and/or proteomic information. The method may also include (g) comparing the initial genetic, transcriptomic, and/or proteomic information, second genetic, transcriptomic, and/or proteomic information, and/or third genetic, transcriptomic, and/or proteomic information, and/or one or more subsequent genetic, transcriptomic, and/or proteomic information to genetic, transcriptomic and/or proteomic information of a different clonal population of cells having a different unique barcode that was subjected to the selective pressure.

In another aspect, a method of identifying a cellular program that facilitates adaptation to a pressure is provided. The method may include: (a) transducing cells with a plurality of barcodes such that each cell contains a single, unique barcode, or barcode combinations; (b) expanding the cells in culture to create a starting cell pool of clones of cells containing each barcode; (c) obtaining first genetic, transcriptomic, and/or proteomic information from a first subset of the starting cell pool; (d) culturing a second subset of the starting cell pool in the presence of a selective pressure to expand the starting cell pool and form an intermediate cell pool; (e) obtaining second genetic, transcriptomic, and/or proteomic information from a first subset of the intermediate cell pool; (f) continuing to culture a second subset of the intermediate cell pool in the presence of the selective pressure to expand the intermediate cell pool and form a final cell pool; (g) obtaining third genetic, transcriptomic, and/or proteomic information from at least a subset of the final cell pool; and (h) quantifying a level of each barcode in the final cell pool, intermediate cell pool, and/or starting cell pool. The method may also include: (i) assigning cells with barcodes enriched in the final cell pool as winning clones and/or assigning cells with barcodes depleted in the final cell pool as losing clones. The method may also include: (j) determining a genetic mutation, transcription program, and/or protein expression associated with at least one winning clone and/or at least one losing clone.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic showing an example overview of longitudinal transcriptomics of barcoded cells (bottom), which combines traditional endpoint studies (top left) with clonal analysis (top right) to allow analysis of response and adaptation of individual cells/clones over time in response to a selective pressure.

FIG. 2 is a schematic showing an example of expression-based high complexity barcoding coupled to single cell analysis for analysis of sensitivity and resistance to a drug treatment (or other selective pressure) over time.

FIG. 3 illustrates an example scRNA-seq compatible barcoding construct with the barcode insertion site, T2A (self-cleaving peptide), and EGFP and puromycin selection genes labeled. Insertion of the construct into a cell results in expression of EGFP transcript containing the barcode. EGFP-barcode transcripts can be sequenced by RNA-seq.

FIGS. 4A and 4B are graphs showing abundance (as log 2) of each barcode on Day 4 (Y axis) compared with at the endpoint (X axis) of treatment with Erlotinib (FIG. 4A) or Degrader (FIG. 4B). Barcodes associated with high levels of abundance at both time points are labeled. Points corresponding to select barcodes are labeled.

FIG. 5 is graph from FIG. 4A, with clones expected to survive (left circle) and clones expected to both survive and adapt (right circle) to Erlotinib circled.

FIG. 6 illustrates an experimental workflow for using this technology to determine pre-existing non-genetic features of EGFR inhibitor resistant cells in PC9 cells (non-small cell lung carcinoma driven by EGFR mutation).

FIG. 7 is a series of phase contrast images over time, monitoring kinetics for apoptosis induction of PC9 cells treated with Erlotinib

FIG. 8 is (left) a heatmap of sample barcode abundance before and after Erlotininb treatment. Barcodes were classified based on their relative abundance changes into resistant and sensitive classes. Differential gene expression analysis were conducted to determine differentially expressed genes between the boxed two barcode classes. Pathway enrichment analysis was then conducted using the differential expressed gene list to determine (right) pathways and their gene members that were statistically significantly enriched in resistant clones.

FIG. 9 is (left) a heatmap of barcode abundance, barcodes were clustered based on their relative abundance changes subject to Erlotininb and EGFR Degrader treatment. Differential gene expression and pathway enrichment analysis between the two boxed barcode classes identified (right) pathways and their gene members that were statistically significantly enriched in EGFR Degrader sensitive clones.

FIG. 10A is (left) a heatmap of barcode abundance, barcodes were clustered based on their relative abundance changes subject to Erlotininb and EGFR degrader treatment. Barcodes that displayed differential responses to Erlotinib vs EGFR Degrader (box barcodes, Degrader resistant but much less Erlotinib resistant) were selected for differential gene expression analysis that compares the transcription responses of these cells to treatment with Erlotinib or Degrader, with (right) gene-associated pathways identified.

FIG. 10B are (left) violin plots illustrating ATF4 and SLC7A5 in untreated, Erlotinib-treated, and Degrader-treated cells, and (right) a potential mechanism of action of Erlotinib in Degrader resistant clones.

FIG. 11A illustrates an experimental strategy for determining the effect of EGFR protein depletion on Erlotinib efficacy using PC9 cells.

FIG. 11B is a bar graph indicating relative gene expression of Integrated Stress Response genes and MAPK genes in PC9 cells upon treatment with siNTC+Erlotinib (100 nM), siEGFR+DMSO, or siEGFR+Erlotinib (100 nM). Gene expression is normalized to siNTC+DMSO. The legend from top to bottom is representative of each group of three bars from left to right.

FIG. 11C is a violin plot illustrating MAPK gene expression in untreated (day 1), Erlotinib treated (day 5 of treatment), or Degrader treated (day 5 of treatment) cells.

FIG. 11D shows images of PC9 cells stained with crystal violet to assess for cell viability upon treatment with siNTC+Erlotinib (100 nM), siEGFR+DMSO, or siEGFR+Erlotinib (100 nM).

FIG. 12 shows images of H1975 cells stained with crystal violet to assess for cell viability upon treatment with 100 nM Osimertinib (left), 100 nM Osimertinib and 1 uM Control compound (middle), or 100 nM Osimertinib+1 uM Degrader (right).

FIG. 13A is a schematic showing pharmacological modulators (Tg: thapsigargin; Tm: tunicamycin) of ER stress leading to induction of the integrated stress response (ISR). Integrated stress response inhibitor (ISRIB) inhibits pelF2α, resulting in inhibition of the ISR.

FIG. 13B is (left) a bar graph indicating % Survival of cells treated with Degrader, Degrader+Tm, or Degrader+Tg and (right) images of cells stained with crystal violet to assess cell viability upon treatment.

FIG. 13C is (left) a bar graph indicating % Survival of cells treated with Degrader, Erlotinib (Erl), or Erl+ISRIB and (right) images of cells stained with crystal violet to assess cell viability upon treatment.

FIG. 14 is a heatmap of cell clone abundance as measured by NGS analysis of barcodes. Blue boxes indicate Degrader-resistant or Erlotinib-resistant clones.

FIG. 15 is a bar graph of ISR gene and MAPK target gene expression (relative to control) as measured by qPCR in cells upon treatment with Erlotinib, Erlotinib and ISRIB, Degrader, or ISRIB alone.

FIG. 16 is a schematic showing validation of mechanistic pathways for EGFR kinase inhibitor efficacy.

FIGS. 17A-17G TraCe-seq reveals loss of efficacy of dual EGFR inhibitor-degrader compared to conventional EGFR kinase inhibitors. FIG. 17A shows a schematic of TraCe-seq workflow that allows for clonal tracking and fitness mapping as a function of time. This allows for longitudinal assessment of gene expression to enable direct comparison of efficacy and mechanisms of response or resistance to different treatments at single-cell resolution. FIG. 17B shows a schematic of TraCe-seq barcoding construct with 8-nt sub-library index and 30-nt GC-optimized barcodes. FIG. 17C shows chemical structures of erlotinib, erlotinib-derived EGFR degrader GNE-104, and non-degrader control GNE-069. FIG. 17D shows a schematic of the TraCe-seq experimental design to compare efficacy and mechanisms of response and resistance to EGFR kinase inhibitors (erlotinib and GNE-069) versus EGFR degrader (GNE-104). Number of cells captured by single-cell RNA sequencing under each condition are shown. FIG. 17E shows a bar graph showing TraCe-seq barcode diversity (Shannon index) before (baseline) and under treatments. Numbers of TraCe-seq barcodes recovered from single-cell RNA-seq reads are shown in each bar. FIG. 17F shows a violin plot showing expression of MAPK pathway signature genes obtained from single cell RNA-seq before (baseline) and under treatments (*p<10⁻¹⁰⁰) FIG. 17G is a bar graph showing fraction of cells in GO cell cycle state, inferred by gene expression, before (baseline) and under treatments.

FIGS. 18A-18H TraCe-seq reveals distinct transcriptional states associated with response and resistance to erlotinib versus erlotinib-derived degrader GNE-104. FIG. 18A shows a heatmap of treatment resistant clones showing their relative abundance before (baseline) and under erlotinib, GNE-069, or GNE-104 treatments. FIG. 18B shows violin plots showing normalized pre-existing expression levels of VIM and AXL in cells belonging to TraCe-seq barcode categories prior to drug treatment. FIG. 18C shows a gene set enrichment analysis showing protein processing in ER genes are significantly depleted in degrader-resistant cells compared to degrader-sensitive cells prior to treatment. FIG. 18D shows a UMAP visualization of all cells treated by erlotinib, GNE-069, and GNE-104. Arrowed lines indicate trajectories derived by Slingshot. FIG. 18E shows a schematic showing relationships between UMAP clusters and trajectories inferred by Slingshot. FIG. 18F shows the trend in various pathway expression outputs plotted as a function of pseudotime along each trajectory. FIG. 18G shows a bar graph showing normalized distribution of kinase-inhibitor resistant versus degrader resistant cells and end-clusters of each trajectory. FIG. 18H shows gene enrichment analysis showing protein processing in ER genes are significantly upregulated under erlotinib treatment compared to GNE-104 treatment in degrader-resistant cells.

FIGS. 19A-19I Validation of the TraCe-seq prediction reveals EGFR protein and activation of integrated stress response genes downstream of ER stress contribute to cytotoxic activity of EGFR kinase inhibitors. FIG. 19A shows a clonogenic assay showing siEGFR promoted survival of PC9 cells under EGFR kinas inhibitors erlotinib and osimertinib treatments. FIG. 19B shows a clonogenic assay showing allosteric degrader GNE-641 but not the non-degrader control GNE-640 promoted survival of NCI-H1975 cells under osimertinib treatment. EGFR and pEGFR levels were analyzed by western blot under the indicated treatment conditions. Cells were pre-treated with osimertinib for 4 hours to ensure near saturation of EGFR proteins by this covalent inhibitor prior to co-treatment with GNE-640 or GNE-641 for 24 hrs. FIG. 19C shows a schematic model showing how inhibitor bound EGFR protein contributes to cytotoxic activity of EGFR kinase inhibitors and how EGFR degradations attenuates such activity. FIGS. 19D, 19E show qRT-PCR quantifications of key pro-death genes downstream of ER stress in PC9 cells, and NCI-H1975 cells, respectively, treated under the indicated conditions for three days. FIG. 19F shows a clonogenic assay showing co-treatment with ISRIB promoted PC9 cell survival under erlotinib treatment. FIGS. 19G, 19H show clonogenic assays showing PERK activator CCT020312 greatly enhanced efficacy of FDA-approved EGFR inhibitors erlotinib and osimertinib in PC9 (19G) and NCI-H1975 (19H) cells. FIG. 19I shows a bar graph comparing growth inhibition by EGFRi+CCT020312 versus the single agents. All error bars represent SD.

FIGS. 20A-20E Quality control metrics for TraCe-seq barcode recovery and assignment. FIG. 20A shows single-cell RNA-seq results obtained from a mixture of five cell lines labeled with five different TraCe-seq barcodes respectively were visualized using UMAP. Clustering was performed based on transcriptomic differences between cells (shown left, UMAP plot labeled by transcriptomic clusters) and annotated by TraCe-seq barcode assigned (shown right, UMAP plot labeled by TraCe-seq barcode). As expected majority of cells of a given TraCe-seq barcode label corresponded to a specific cell line/cluster. FIG. 20B shows heatmaps of top 3 marker genes form each cluster based on TraCe-seq annotation in bulk RNA-seq (left panel) and scRNA (right panel) support TraCe-seq labeling can recover major transcriptional features of each barcoded population. FIG. 20C shows violin plots showing marker gene expression of individual genes in each cell line among the mixture. FIG. 20D left panel shows FACS enrichment for the top 50% eGFP-expressing cells. The four panels on the right show dropout rate of TraCe-seq barcodes compared to endogenously expressed genes before and after FACS sorting in NCI-H1373 and PC9 cells. In these plots, the red points represent the TraCe-seq barcodes, and the blue points represent groups of endogenous genes with the same median dropout rate for the endogenous genes. FIG. 20E is a box plot showing cells with mis-assigned TraCe-seq barcodes had significantly lower barcode-expression levels. The bottom side of the box represents the first quartile, and the top side, the third quartile. The line represents the median.

FIGS. 21A-21D. FIG. 21A shows a western blot showing the does-dependent EGFR degradation induced by GNE-104 in HCC827 cells. FIG. 21B is a western blot showing effect on EGFR and pEGFR by GNE-104 versus GNE-069 in HCC87 cells. FIG. 21C is a western blot showing excess free VHL ligand inhibits GNE-104 induced EGFR protein degradation in HCC827 cells, while no effects were observed for GNE-069. FIG. 21D is a characterization of the biochemical potency (towards common EGFR mutant variants) selectivity (against a panel of 218 kinases) of GNE-104 and GNE-069 in an in vitro kinase inhibition assay.

FIGS. 22A-22D Characterization of the response of PC9 cells to erlotinib, GNE-069, and GNE-104. FIG. 22A shows a dendogram showing TraCe-seq barcode enrichment patterns in replicates across 500 different PC9 clones subject to erlotinib (2 μM) or GNE (1 μM) treatment over two months across replicates. Barcode enrichment were highly reproducible within individual treatment and differed between erlotinib and GNE-104 treatments. FIGS. 22B-22D are a comparison of the anti-growth effects of erlotinib, GNE-069, and GNE-104 in PC9 cells by clonogenic assay (22B), cell counting (22C) and Incucyte imagining (22D). All three compounds were applied at 1 μM. Erlotinib and GNE-069 had comparable activity by all three measures, whereas GNE-104 was less efficacious.

FIGS. 23A-23D TraCe-seq barcode enrichment and depletion analysis. FIG. 23A shows deep NGS sequencing of TraCe-seq clonal abundance derived from genomic DNA (x-axis) correlates with scRNA-seq derived TraCe-seq clonal abundance (y-axis). Cutoff (line) shown of minimum TraCe-seq barcode for downstream clonal depletion analysis shown in FIG. 23D. FIG. 23B shows a comparison of TrCe-seq clonal abundance under different treatments. Points are number of cells with a give TraCe-seq barcode under indicated conditions. Pearson correlation coefficient of the barcode abundance distributions are shown. FIG. 23C shows a distribution of log 2-fold change of TraCe-seq barcode in each treatment condition compared to baseline. FIG. 23D is a heatmap showing relative abundance of depleted TraCe-seq barcodes upon erlotinib, GNE-069, or GNE-104 treatments.

FIGS. 24A-24C. FIG. 24A Clonogenic assay confirming differential activity of EGFR kinase inhibitors versus degrader GNE-104. FIG. 24A is a clonogenic assay showing differential anti-growth effects of GNE-104 compared to erlotinib or GNE-069 across four different EGFR-mutant lung cancer cell lines. FIG. 24B shows quantification of relative viability of the same four EGFR-mutant lung cancer cell lines shown in panel (a) under degrader GNE-104 or non-degrader control GNE-069 treatment relative to erlotinib over 14 days using CellTiter-Glo luminescent cell viability assay. FIG. 24C shows clonogenic assay showing that high concentration of free VHL ligand (10 μM) did not affect cellular response to erlotinib or GNE-069 in PC9 cells. The VHL inactive enantiomer was induced as a further control.

FIGS. 25A-25C Abundance of treatment resistant versus sensitive TraCe-seq barcodes among inferred trajectories. FIG. 25A is a density plot showing distribution of cells with kinase inhibitor resistant versus kinase inhibitor sensitive TraCe-seq barcodes in the UMAP space subject to erlotinib/GNE-069 treatment. Each grey dot represents an individual cell. FIG. 25B shows a comparison of resistant versus sensitive barcode category distributions at the end UMAP cluster of the four inferred trajectories. Path a, b, and c were each dominated by at least one resistant clone category (inferred to represent adaptation/resistance), while Path d had the highest relative abundance of drug sensitive clones thus were inferred to represent response. FIG. 25C is a density plot showing distribution of cells with degrader resistant barcodes subject to GNE-104 treatment versus kinase inhibitor erlotinib/GNE-069 treatments. Each grey dot represents an individual cell.

FIGS. 26A-26G. Additional characterization of cells treated with siEGFR and kinase inhibitors or osimertinib plus allosteric EGFR degrader. FIG. 26A shows a schematic showing the siEGFR experimental setup in PC9 and HCC4006 cells. FIG. 26B is a western blot analysis of total EGFR and pEGFR subject to the indicated treatment for two days. FIG. 26C is qRT-PCR analysis of key MAPK transcriptional targets in PC9 cells treated under indicated conditions for three days. FIG. 26D is qRT-PCT analysis of key transcriptional targets of the MAPK pathway in HCC4006 cells under indicated conditions for five days. FIG. 26E is a clonogenic assay showing siEGFR promoted survival of HCC4006 cells under EGFR kinase inhibitors erlotinib and osimertinib treatments. FIG. 26F shows qRT-PCR analysis of key pro-death genes downstream of ER stress in HCC4006 cells under indicated conditions on day five. FIG. 26G shows qRT-PCR analysis of key transcriptional targets of the MAPK pathway in NCI-H1975 cells under indicated conditions on day 3. N.D.: not detected. All error bars represent S.D.

FIGS. 27A-27E Characterization of GNE-640 and GNE-641. FIG. 27A shows the chemical structures of allosteric EGFR ligand EAI-045, GNE-641 (active degrader), and GNE-640 (inactive degrader). FIG. 27B is a western blot showing that addition of the free VHL ligand can rescue the EGFR degradation upon GNE-641 treatment in NCI-H1975 cells. GNE-640 was included as a further control. FIG. 27C shows a clonogenic assay showing very modest single agent activity of EAI-045, GNE-640, or GNE-641 in NCI-H1975 cells compared to osimertinib. FIG. 27D is quantification showing relative viability of NCI-H1975 cells co-treated with 0.1 μM osimertinib alone over 14 days using CellTiter-Glo luminescent cell viability assay. Viability is normalized to osimertinib only. FIG. 27E is a clonogenic assay showing allosteric degrader GNE-641 but not the degrader control GNE-640 promoted survival of NCI-H3255 cells under osimertinib treatment.

FIGS. 28A-28G Pharmacological modulation of ER stress alters response to EGFR kinase inhibitors and degrader. FIG. 28A is a schematic showing pharmacological modulators of ER stress pathway. FIG. 28B is the qRT-PCR quantification of pro-death genes downstream of ER stress and key transcriptional targets of the MAPK pathway in PC9 cells under indicated treatment conditions on day four. FIG. 28C is a clonogenic assay showing attenuation of osimertinib activity by ISRIB in NCI-H1975 cells. FIG. 28D is qRT-PCR quantification of pro-death integrated stress response genes downstream of ER stress and key transcriptional targets of the MAPK pathway in NCI-H1975 cells under indicated treatment conditions on day three. N.D.: not detected. FIG. 28F is the quantitative comparisons of efficacy changes of erlotinib versus GNE-104 subject to co-treatment with indicated pharmacological ER stress modulators in PC9 cells using CellTiter-Glo luminescent cell viability assay on day five. FIG. 28G is qRT-PCR quantifications of key pro-death genes downstream of ER stress in PC9 cells treated under the indicated conditions on day three. All error bars are S.D.

FIGS. 29A-29D PERK activator CCT020312 potentiates activity of FDA-approved EGFR kinase inhibitors. FIGS. 29A-29C are qRT-PCR quantification of pro-death genes downstream of ER stress and MAPK target genes in PC9 cells (29A-29B) and NCI-H1975 cells (29C) under the indicated conditions on day three. FIG. 29D is quantification showing EGFRi+CCT020312 combinations were more effective in inducing cell death compared to the single agents using Caspase-Glo3/7 assay system. All error bars represent S.D.

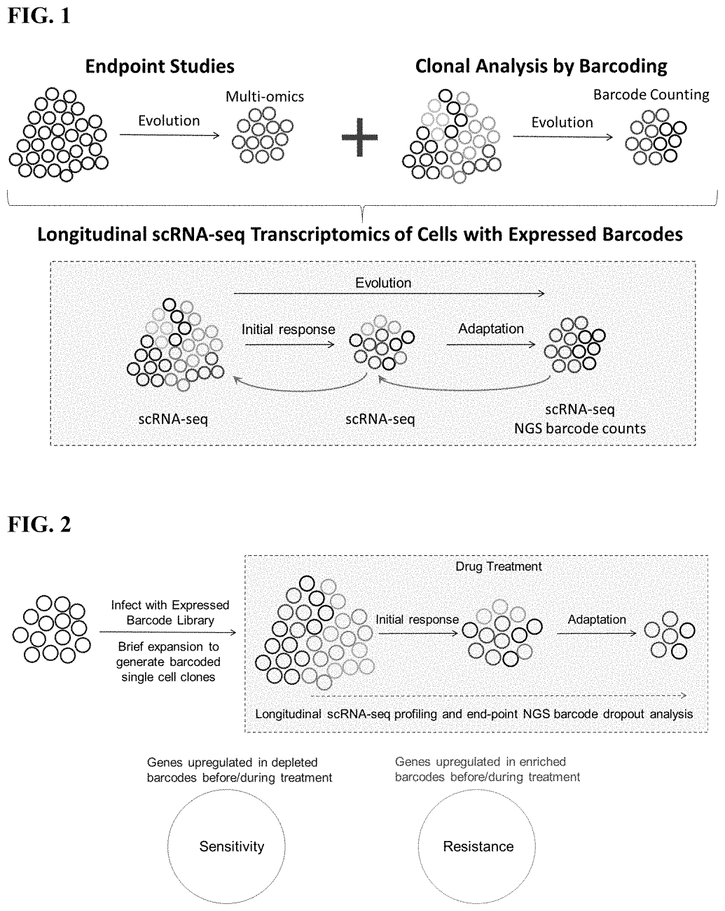

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure

A “barcode” refers to one or more nucleotide sequences that are used to identify a cell or clonal population with which the barcode is associated. Barcodes can be 3-1000 or more nucleotides in length, preferably 10-250 nucleotides in length, and more preferably 10-30 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. A barcode is “unique” when the barcode is (statistically) present in about one cell in a population of cells. The cell containing the bar code can then be expanded to make a clonal population, such that each cell of the clonal population contains the same barcode. For example, “a plurality of barcoded cells, wherein each barcoded cell comprises a single, unique barcode” may refer to a population of cells which contains (statistically) a single cell containing a given barcode or a unique combination of barcodes. Alternatively, it may refer to a population of cells which contains a plurality of clonal populations of cells, each cell of each clonal population containing the same barcode, but cells of different clonal populations containing different barcodes.

The term “sensitive” or “sensitivity” is used herein to refer to the responsiveness of a cell or a population of cells to a selection pressure or therapeutic agent. Cell responsiveness may be growth arrest, quiescence, senescence, apoptosis, or other forms of programmed cell death. Cell responsiveness may be the intended response to the selection pressure or therapeutic agent; for example, cell apoptosis in response to a cytotoxic agent. Cell responsiveness may be changes in cellular properties induced by the selection pressure, including cell fate. Cell responsiveness may be cell plasticity, cellular reprogramming, growth kinetics or metastatic potential. In embodiments, the selection pressure may be treatment with a therapeutic agent, contact with a contaminant, genomic engineering, engraftment into a host, a culture condition, a growth condition, contact with a stimulus, or contact with other cells. In embodiments, the therapeutic agent may be a cancer therapeutic (e.g. a kinase inhibitor or other chemotherapeutic agent).

The term “resistance” is used herein to refer to lack of intended response of a cell or a population of cells to a selection pressure or therapeutic agent. In embodiments, resistance may be adaptation to a selection pressure or therapeutic agent. In embodiments, resistance may be due to one or more pre-existing features of a cell or population of cells. In embodiments, resistance may be acquired, for example by activation of a survival pathway in response to a selection pressure or therapeutic agent. For example, resistance may include adaptation of a cancer cell to a cancer therapeutic, resulting in cancer cell survival.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Methods

Without being bound by theory, it is expected that the methods described herein will capture cellular population heterogeneity with clonal granularity. The methods herein may allow simultaneous monitoring of sensitive and resistant clonal responses to treatments or conditions, and/or identification of pre-existing features that confer resistance without the need of establishing fully resistant cells. Further, the methods described herein may allow comparison of the mechanism of action (MOA) of different molecular entities in heterogeneous populations at cell clone resolution, and/or tracking of evolutionary trajectories of cells. In addition to oncology, the methods may be applied to other disease areas for characterization of cell evolution processes of interest, including but not limited to cellular reprogramming and cell engineering.

For example, a plurality of cells, each cell containing a label (e.g., barcode) to identify a clone or clonal population of cells, are sequenced at a first time-point before undergoing a selective pressure. The cells are then subjected to the selective pressure for a period of time, after which the surviving cells are sequenced. The cells are further subjected to the selective pressure for another period of time, after which a third sequencing procedure is performed. The cells may be subjected to the same selective pressure, followed by sequencing, one or more additional times. The cells may be subjected to a different selective pressure (with or without additional sequencing steps) after the first. For example, cells that survive the first selective pressure may be subjected to a second selective pressure to determine whether the surviving cells are sensitive or resistant to the second selective pressure. The cells may be subjected to multiple subjective pressures at the same time, for example to test a co-treatment therapy.

The data from the sequencing steps can then be analyzed to determine what traits of the cells make them prone to survival, adaptation, and/or sensitivity to the selective pressure(s). For example, a cell having increased (or decreased) expression of a particular RNA (or protein, or presence of a gene mutation/allele/epigenetic profile, etc.) may be more likely to survive and/or thrive in the presence of the selective pressure. The barcode corresponding to that cell may be expected to be present in higher abundance in the surviving cells after exposure to the selective pressure than the barcode of a cell that does not contain that level of expression (or presence). Conversely, the barcode corresponding to a cell that contains a trait that makes it less likely to survive and/or thrive in the presence of the selective pressure may be expected to be present in lower abundance (or absent) in the surviving cells after exposure to the selective pressure.

In an aspect is provided a method for screening cells for a trait. The method may include: (a) obtaining a plurality of barcoded cells, wherein each barcoded cell includes a single, unique barcode; (b) performing a first sequencing of RNA and/or DNA on a subset of the plurality of barcoded cells; (c) culturing the plurality of barcoded cells in the presence of a selection pressure for a first period of time, thereby forming a first plurality of cells; (d) performing a second sequencing of RNA and/or DNA on a subset of the first plurality of cells; (e) culturing the first plurality of cells in the presence of the selection pressure for a second period of time, thereby forming a second plurality of cells; (f) performing a third sequencing of RNA and/or DNA on at least a subset of the second plurality of cells; and (g) determining a level of a barcode sequenced in the first sequencing, second sequencing, and/or third sequencing. In embodiments, the single, unique barcode is a unique combination of barcodes.

In another aspect is provided a method for screening cells for a response trait to a therapeutic agent. The method may include: (a) obtaining a plurality of barcoded cells, wherein each barcoded cell comprises a single, unique barcode; (b) performing a first sequencing of RNA and/or DNA on a subset of the plurality of barcoded cells; (c) culturing the plurality of barcoded cells in the presence of the therapeutic agent for a first period of time, thereby forming a first plurality of cells; (d) performing a second sequencing of RNA and/or DNA on a subset of the first plurality of cells; (e) culturing the first plurality of cells with the therapeutic agent for a second period of time, thereby forming a second plurality of cells; (0 performing a third sequencing of RNA and/or DNA on a subset of the second plurality of cells; and (g) determining a level of a barcode sequenced in the first sequencing, second sequencing, and/or third sequencing. In embodiments, the single, unique barcode is a unique combination of barcodes.

For the methods provided herein, in embodiments, the plurality of barcoded cells of step (a) is expanded in culture prior to step (b) or step (c). In embodiments, the plurality of barcoded cells had been expanded in culture prior to step (a).

In embodiments, steps (e) and (f) are repeated for one or more iterations, thereby forming one or more subsequent pluralities of cells and performing one or more subsequent sequencings on a subset(s) of the subsequent pluralities of cells. In embodiments, step (g) further includes determining levels of barcodes sequenced in the one or more subsequent sequencings.

In embodiments, the plurality of barcoded cells includes a plurality of clonal populations, wherein each cell within a single clonal population includes the same single, unique barcode. In embodiments, the relative abundance of cells in each clonal population is approximately equal to the number of cells in each other clonal population in step (a). In embodiments, the relative abundance of cells in each clonal population is determined as relative to the number of cells containing each barcode as determined in the first sequencing step. In embodiments, the single, unique barcode is a unique combination of barcodes.

For the methods provided herein, a level of two or more barcodes sequenced in the first sequencing, second sequencing, and or third sequencing may be determined. In embodiments, a level of two or more barcodes sequenced in the first sequencing, second sequencing, and/or third sequencing are determined. In embodiments, a level of two or more barcodes sequenced in the first sequencing and second sequencing are determined. In embodiments, a level of two or more barcodes sequenced in the second sequencing and third sequencing are determined. In embodiments, a level of two or more barcodes sequenced in the first sequencing and third sequencing are determined. A level of a barcode may be any determination of an amount of the barcode, for example abundance of the barcode, relative abundance, etc.

In embodiments, the methods provided herein further include identifying a barcode(s) that is enriched in the first plurality of cells and/or the second plurality of cells and/or one or more subsequent pluralities of cells. In embodiments, the barcode(s) is enriched compared to one or more other barcodes in the first plurality of cells and/or the second plurality of cells and/or one or more subsequent pluralities of cells. In embodiments, the barcode(s) is enriched compared to the amount of the barcode(s) in the plurality of barcoded cells.

In embodiments, the methods provided herein further include identifying one or more genes having higher levels of expression (and/or presence of a trait such as a gene mutation/allele/epigenetic profile, etc.) in cells comprising the enriched barcode(s). In embodiments, the identifying the one or more genes (or presence of the trait) includes determining that the level of expression of at least one gene is higher (or the trait is present or more likely to be present) in the first sequencing, second sequencing, third sequencing, and/or one or more subsequent sequencings in the cells containing the enriched barcode(s) compared to cells containing a different barcode(s) that had a different level of enrichment or no enrichment. In embodiments, the identifying includes identifying an adaptive trait based upon higher expression of the at least one gene (or increased presence of the trait). For example, a cellular pathway may be implicated by the up- and/or down-regulation of multiple genes involved in that pathway. In embodiments, the identifying includes identifying that the gene (or trait) is involved in adaptation of the cell to the selection pressure or therapeutic agent based upon higher expression of the at least one gene (or increased presence of the trait).

In embodiments, the methods provided herein further include identifying one or more genes having lower levels of expression (and/or absence of a trait such as a gene mutation/allele/epigenetic profile, etc.) in cells containing the enriched barcode(s) compared to cells containing a different barcode(s) that had a different level of enrichment. In embodiments, the identifying one or more genes having lower levels of expression (or absence of the trait) includes determining the level of expression of at least one gene is lower (or the trait is absent or less likely to be present) in the first sequencing, second sequencing, third sequencing, and/or one or more subsequent sequencings in the cells containing the enriched barcode(s) compared to cells comprising a different barcode(s) that had a different level of enrichment or no enrichment. In embodiments, the identifying includes identifying an adaptive trait based upon lower expression of the at least one gene (or absence of the trait). In embodiments, the identifying includes determining that the gene (or trait) is involved in adaptation of the cell to the selection pressure or therapeutic agent based upon lower expression of the at least one gene (or absence of the trait).

In embodiments, the methods provided herein further include: (h) identifying a barcode(s) that is enriched in the first plurality of cells and/or the second plurality of cells and/or one or more subsequent pluralities of cells; (i) identifying from the first sequencing a first gene having a higher level of expression in cells containing the enriched barcode(s) than in cells containing a barcode(s) that is not enriched in the first plurality of cells and/or the second plurality of cells and/or one or more subsequent pluralities of cells, such that the higher expression of the first gene indicates that the first gene is a candidate pre-existing trait for resistance or sensitivity to the selective pressure or therapeutic agent. In embodiments, the presence or absence of a trait is determined rather than expression of a gene.

In embodiments, the methods provided herein further include: (j) identifying a barcode(s) that is enriched in the first plurality of cells and/or the second plurality of cells and/or one or more subsequent pluralities of cells; (k) identifying from the first sequencing a second gene having a lower level of expression in cells containing the enriched barcode(s) than in cells containing a barcode(s) that is not enriched in the first plurality of cells and/or the second plurality of cells and or one or more subsequent pluralities of cells, such that the lower expression of the second gene indicates that the second gene is a candidate pre-existing trait for resistance or sensitivity to the selective pressure or therapeutic agent. In embodiments, the presence or absence of a trait is determined rather than expression of a gene.

In embodiments, the methods provided herein further include: (l) identifying a barcode that is enriched in the first plurality of cells and/or the second plurality of cells and/or one or more subsequent pluralities of cells; (m) identifying from the second sequencing a third gene having a higher level of expression in cells containing the enriched barcode(s) than in cells containing a barcode that is not enriched in the first plurality of cells and/or the second plurality of cells and/or one or more subsequent pluralities of cells, such that the higher expression of the third gene indicates that the third gene is a candidate adaptive trait for resistance or sensitivity to the selective pressure or therapeutic agent. In embodiments, the expression of the third gene is not higher in cells containing a barcode(s) that is not enriched in the first plurality of cells and/or the second plurality of cells and/or one more subsequent pluralities of cells. In embodiments, the presence or absence of a trait is determined rather than expression of a gene.

In embodiments, the methods provided herein further include: (n) identifying a barcode(s) that is enriched in the first plurality of cells and/or the second plurality of cells and/or one more subsequent pluralities of cells; (o) identifying from the second sequencing and/or the third sequencing a fourth gene having a lower level of expression in cells containing the enriched barcode(s) than in cells containing a barcode(s) that is not enriched in the first plurality of cells and/or the second plurality of cells and/or one or more subsequent pluralities of cells, such that the lower expression of the fourth gene indicates that the first gene is a candidate adaptive trait for resistance or sensitivity to the selective pressure or therapeutic agent. In embodiments, the expression of the fourth gene is not lower in cells containing a barcode(s) that is not enriched in the first plurality of cells and/or the second plurality of cells. In embodiments, the presence or absence of a trait is determined rather than expression of a gene.

In embodiments, for the methods provided herein, the one or more genes comprise a gene set. In embodiments, the gene set may be a gene set for tyrosine kinase inhibitor resistance, gap junction, pathological Escherichia coli infection, Hepatocellular carcinoma, DNA replication, carbon metabolism, cell cycle, glyoxylate and dicarboxylate metabolism, estrogen signaling pathway, fluid shear stress and atherosclerosis, histidine metabolism, Eptein-Barr virus infection, protein processing in the ER, metabolic pathways, lysosome, focal adhesion, ECM-receptor interaction, small cell lung cancer, apoptosis and/or integrated stress response.

In embodiments, for the methods provided herein, the unique barcode may or may not be integrated into the genome of the barcoded cell. In embodiments, the unique barcode is integrated into the genome of the barcoded cell. In embodiments, the unique barcode is not integrated into the genome of the barcoded cell. In embodiments, the barcode is expressed by the cell. For example, the barcode may be adjacent to a coding sequence such that it is expressed along with the coding sequence. The barcode may be expressed as an RNA. In embodiments, the amount of a barcode in a population of cells is determined based on the abundance of barcode sequences as determined by NGS. In embodiments, the barcode is not expressed by the cell. For example, the amount of a barcode in a population of cells may be determined based on DNA sequencing or other DNA analysis (e.g., PCR) for the presence/absence/amount of the barcode DNA in the cell population. For example, the amount of a barcode in a population may be determined based on a DNA analysis that is not DNA sequencing or conduction DNA sequencing. In embodiments, the unique barcode is a unique combination of barcodes.

For the methods provided herein, the first sequencing, second sequencing, third sequencing, and/or one or more subsequent sequencings may be performed using RNA-seq, DNA sequencing, epigenetic sequencing, or protein sequencing. In embodiments, the first sequencing, second sequencing, third sequencing, and/or one or more subsequent sequencings is performed using next-generation sequencing (NGS). In embodiments, the first sequencing, second sequencing, third sequencing, and/or one or more subsequent sequencings is performed using RNA-seq. In embodiments, the RNA-seq is single cell RNA-seq. In embodiments, the first sequencing, second sequencing, third sequencing, and/or one or more subsequent sequencings is DNA sequencing. In embodiments, the first sequencing, second sequencing, third sequencing, and/or one or more subsequent sequencings is epigenetic sequencing. In embodiments, the first sequencing, second sequencing, third sequencing, and/or one or more subsequent sequencings is protein sequencing. In embodiments, for the methods provided herein, the first sequencing, second sequencing, third sequencing, and/or one or more subsequent sequencings may exclude any one of the sequencing methods recited herein. In embodiments, the first sequencing, second sequencing, third sequencing, and/or one or more subsequent sequencings is not performed using one or more of RNA-seq, DNA sequencing, epigenetic sequencing, or protein sequencing. In embodiments, the first sequencing, second sequencing, third sequencing, and/or one or more subsequent sequencings is not RNA-seq. In embodiments, the first sequencing, second sequencing, third sequencing, and/or one or more subsequent sequencings is not DNA sequencing. In embodiments, the first sequencing, second sequencing, third sequencing, and/or one or more subsequent sequencings is not epigenetic sequencing. In embodiments, the first sequencing, second sequencing, third sequencing, and/or one or more subsequent sequencings is not protein sequencing. In embodiments, the first sequencing, second sequencing, third sequencing, and one or more subsequence sequencings is not conduction DNA sequencing. In embodiments, the first sequencing is not conduction DNA sequencing. In embodiments the second sequencing is not conduction DNA sequencing. In embodiments, the third sequencing is not conduction DNA sequencing. In embodiments, the one or more subsequent sequencings is not conduction DNA sequencing.

In embodiments, for the methods provided herein the selective pressure is treatment with a therapeutic agent, contact with a contaminant, genomic engineering, engraftment into a host, a culture condition, a growth condition, contact with a stimulus, or contact with other cells. In embodiments, the selective pressure is treatment with a therapeutic agent. In embodiments, the selective pressure is contact with a contaminant. In embodiments, the selective pressure is genomic engineering. In embodiments, the selective pressure is engraftment into a host. In embodiments, the selective pressure is a culture condition. In embodiments, the selective pressure is a growth condition. In embodiments, the selective pressure is contact with a stimulus. In embodiments, the selective pressure is contact with other cells. In embodiments, one or more selective pressures may be excluded.

In embodiments, for the methods provided herein, the first period of time is between about 30 minutes and about 1 month. In embodiments, the first period of time is about 30 minutes. In embodiments, the first period of time is about 1 hour. In embodiments, the first period of time is about 5 hours. In embodiments, the first period of time is about 10 hours. In embodiments, the first period of time is about 15 hours. In embodiments, the first period of time is about 20 hours. In embodiments, the first period of time is about 1 day. In embodiments, the first period of time is about 2 days. In embodiments, the first period of time is about 3 days. In embodiments, the first period of time is about 4 days. In embodiments, the first period of time is about 5 days. In embodiments, the first period of time is about 6 days. In embodiments, the first period of time is about 7 days. In embodiments, the first period of time is about 8 days. In embodiments, the first period of time is about 9 days. In embodiments, the first period of time is about 10 days. In embodiments, the first period of time is about 11 days. In embodiments, the first period of time is about 12 days. In embodiments, the first period of time is about 13 days. In embodiments, the first period of time is about 14 days. In embodiments, the first period of time is about 15 days. In embodiments, the first period of time is about 16 days. In embodiments, the first period of time is about 17 days. In embodiments, the first period of time is about 18 days. In embodiments, the first period of time is about 19 days. In embodiments, the first period of time is about 20 days. In embodiments, the first period of time is about 21 days. In embodiments, the first period of time is about 22 days. In embodiments, the first period of time is about 23 days. In embodiments, the first period of time is about 24 days. In embodiments, the first period of time is about 25 days. In embodiments, the first period of time is about 26 days. In embodiments, the first period of time is about 27 days. In embodiments, the first period of time is about 28 days. In embodiments, the first period of time is about 29 days. In embodiments, the first period of time is about 30 days. In embodiments, the first period of time is about 1 month. In embodiments, the first period of time is more than 1 month. In embodiments, the first period of time is about 30 minutes, about 1 hour, about 5 hours, about 10 hours, about 15 hours, about 20 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 1 month. The amount of time may be any value or subrange within ranges provided herein, including endpoints.

In embodiments, for the methods provided herein, the second period of time is between about 12 hours and 12 months. In embodiments, the second period of time is about 12 hours. In embodiments, the second period of time is about 1 day. In embodiments, the second period of time is about 5 days. In embodiments, the second period of time is about 10 days. In embodiments, the second period of time is about 15 days. In embodiments, the second period of time is about 20 days. In embodiments, the second period of time is about 25 days. In embodiments, the second period of time is about 1 month. In embodiments, the second period of time is about 1.5 months. In embodiments, the second period of time is about 2 months. In embodiments, the second period of time is about 2.5 months. In embodiments, the second period of time is about 3 months. In embodiments, the second period of time is about 3.5 months. In embodiments, the second period of time is about 4 months. In embodiments, the second period of time is about 4.5 months. In embodiments, the second period of time is about 5 months. In embodiments, the second period of time is about 5.5 months. In embodiments, the second period of time is about 6 months. In embodiments, the second period of time is about 6.5 months. In embodiments, the second period of time is about 7 months. In embodiments, the second period of time is about 7.5 months. In embodiments, the second period of time is about 8 months. In embodiments, the second period of time is about 8.5 months. In embodiments, the second period of time is about 9 months. In embodiments, the second period of time is about 9.5 months. In embodiments, the second period of time is about 10 months. In embodiments, the second period of time is about 10.5 months. In embodiments, the second period of time is about 11 months. In embodiments, the second period of time is about 11.5 months. In embodiments, the second period of time is about 12 months. In embodiments, the second period of time is about 12 hours, about 1 day, about 5 days, about 10 days, about 15 days, about 20 days, about 25 days, about 1 month, about 1.5 month, about 2 months, about 2.5 months, about 3 months, about 3.5 months, about 4 months, about 4.5 months, about 5 months, about 5.5 months, about 6 months, about 6.5 months, about 7 months, about 7.5 months, about 8 months, about 8.5 months, about 9 months, about 9.5 months, about 10 months, about 10.5 months, about 11 months, about 11.5 months, or about 12 months. In embodiments, the second period of time is more than 1 year. The amount of time may be any value or subrange within ranges provided herein, including endpoints.

In an aspect is provided a method for comparing responses to selective pressures. The method includes: (a) obtaining a first plurality of barcoded cells, wherein each barcoded cell includes a single, unique barcode; (b) obtaining a second plurality of barcoded cells that is substantially similar to the first plurality of barcoded cells; (c) performing a first sequencing of RNA and/or DNA from the first plurality of barcoded cells and/or the second plurality of barcoded cells; (d) culturing the first plurality of barcoded cells in the presence of a first selection pressure, thereby forming a first plurality of cells; (e) culturing the second plurality of barcoded cells in the presence of a second selection pressure, thereby forming a second plurality of cells; (f) performing a second sequencing of RNA and/or DNA from the first plurality of cells and/or the second plurality of cells; (g) culturing the first plurality of cells in the presence of the first selection pressure, thereby forming a third plurality of cells; (h) culturing the second plurality of cells in the presence of the second selection pressure, thereby forming a fourth plurality of cells; (i) performing a third sequencing of RNA and/or DNA from the third plurality of cells and/or the fourth plurality of cells; and (j) determining a level of one or more barcodes sequenced in the first sequencing, second sequencing, and/or third sequencing. In embodiments, steps (g) to (i) are repeated for one or more iterations, thereby forming one or more subsequent pluralities of cells and one or more subsequent sequencings. In embodiments, step (j) further comprises determining a level of one or more barcodes in the one or more subsequent sequencing steps. In embodiments, the single, unique barcode is a unique combination of barcodes. In embodiments, for the methods provided herein, the first sequencing, second sequencing, third sequencing, and/or one or more subsequent sequencings may exclude any one of the sequencing methods recited herein. In embodiments, the first sequencing, second sequencing, third sequencing, and/or one or more subsequent sequencings is not performed using one or more of RNA-seq, DNA sequencing, epigenetic sequencing, or protein sequencing. In embodiments, the first sequencing, second sequencing, third sequencing, and/or one or more subsequent sequencings is not RNA-seq. In embodiments, the first sequencing, second sequencing, third sequencing, and/or one or more subsequent sequencings is not DNA sequencing. In embodiments, the first sequencing, second sequencing, third sequencing, and/or one or more subsequent sequencings is not epigenetic sequencing. In embodiments, the first sequencing, second sequencing, third sequencing, and/or one or more subsequent sequencings is not protein sequencing. In embodiments, the first sequencing, second sequencing, third sequencing, and one or more subsequence sequencings is not conduction DNA sequencing. In embodiments, the first sequencing is not conduction DNA sequencing. In embodiments the second sequencing is not conduction DNA sequencing. In embodiments, the third sequencing is not conduction DNA sequencing. In embodiments, the one or more subsequent sequencings is not conduction DNA sequencing.

In embodiments, for the method provided herein, the first plurality of barcoded cells and the second plurality of barcoded cells include a plurality of clonal populations, wherein each cell within a single clonal population includes the same single, unique barcode. In embodiments, the relative abundance of cells in each clonal population is approximately equal to the number of cells in each other clonal population in steps (a) and (b). In embodiments, from the first sequencing step the relative abundance of cells in a clonal population is determined relative to the number of cells containing the barcode(s). In embodiments, the single, unique barcode is a unique combination of barcodes.

In embodiments, the method provided herein further includes determining a first level of expression of a gene in cells having a barcode(s) enriched in the first plurality of cells and/or third plurality of cells and/or one or more subsequent pluralities of cells based on the second sequencing and/or third sequencing and/or one or more subsequent sequencings, and a second level of expression of the gene in the second plurality of barcoded cells, and/or fourth plurality of cells and/or one or more subsequent pluralities of cells based on the first sequencing, second sequencing, and/or third sequencing and/or one or more subsequent sequencings. In embodiments, the method further includes comparing the first level of expression of the gene to the second level of expression of the gene. In embodiments, the presence or absence of a trait is determined rather than expression of a gene.

In an aspect, a method of screening cells for a trait in a cell is provided. The method includes: (a) providing a mixture of cells comprising multiple clonal populations wherein each clonal population comprises an identifier that is unique to the respective clonal populations, and wherein initial genetic, transcriptomic, and/or proteomic information of at least one representative member of each clonal population is known; (b) culturing the mixture of cells in the presence of a first selective pressure for a first period of time, and at the end of the first period of time, obtaining second genetic, transcriptomic, and/or proteomic information for at least one member of a surviving clonal population from within the mixture of cells; (c) subjecting the mixture of cells that were subjected to the first selective pressure to a second selective pressure for a second period of time, and at the end of the second period of time, obtaining third genetic, transcriptomic, and/or proteomic information of at least one member of a surviving clonal population from within the mixture of cells; and (d) determining the level of a clonal population present in the final mixture of cells based upon the unique identifier for the clonal population. In embodiments, step (b) is repeated for one or more iterations. In embodiments, step (c) is repeated for one or more iterations. In embodiments, steps (b) and (c) are repeated for one or more iterations.

In embodiments, the method provided herein further includes identifying an adaptive trait, wherein the adaptive trait is a genetic and/or proteomic trait present in or absent from a clonal population in the final mixture of cells. In embodiments, the adaptive trait is a presence or absence of a gene, allele, genetic modification, transcript, or protein; or a change in a gene, allele, transcript, or protein when comparing the first, second and/or third and/or one or more subsequent genetic, transcriptomic, and/or proteomic information obtained. In embodiments, the adaptive trait is the presence of a gene, allele, genetic modification, transcript, or protein. In embodiments, the adaptive trait is an absence of a gene, allele, genetic modification, transcript, or protein. In embodiments, the adaptive trait is a change in a gene, allele, transcript, or protein when comparing the first, second and/or third and/or one or more subsequent genetic, transcriptomic, and/or proteomic information obtained.

In embodiments, step (b) includes obtaining fourth genetic, transcriptomic, and/or proteomic information of at least one member of a second surviving clonal population from within the mixture of cells. In embodiments, step (c) comprises obtaining fifth genetic, transcriptomic, and/or proteomic information of at least one member of a second surviving clonal population from within the mixture of cells.

In embodiments, the method further includes comparing information from the initial genetic, transcriptomic, and/or proteomic information, second genetic, transcriptomic, and/or proteomic information, and/or third genetic, transcriptomic, and/or proteomic information, and/or one or more subsequent genetic, transcriptomic, and/or proteomic information. In embodiments, the method further includes comparing the initial genetic, transcriptomic, and/or proteomic information, second genetic, transcriptomic, and/or proteomic information, and/or third genetic, transcriptomic, and/or proteomic information, and/or one or more subsequent genetic, transcriptomic, and/or proteomic information to genetic, transcriptomic and/or proteomic information of a different clonal population of cells having a different unique barcode that was subjected to the selective pressure. In embodiments, the method further includes comparing the initial genetic, transcriptomic, and/or proteomic information, second genetic, transcriptomic, and/or proteomic information, and/or third genetic, transcriptomic, and/or proteomic information, and/or one or more subsequent genetic, transcriptomic, and/or proteomic information from a clonal population of cells having a unique barcode, to genetic, transcriptomic and/or proteomic information from a different determination step for the clonal population of cells. In embodiments, the unique barcode is a unique combination of barcodes.

In embodiments, for the method provided herein, obtaining the genetic, transcriptomic, and/or proteomic information includes RNA-seq, DNA sequencing, epigenetic sequencing, or protein sequencing. In embodiments, obtaining the genetic, transcriptomic, and/or proteomic information includes NGS. In embodiments, obtaining the genetic, transcriptomic, and/or proteomic information includes RNA-seq. In embodiments, the RNA-seq is single cell RNA-seq. In embodiments, obtaining the genetic, transcriptomic, and/or proteomic information includes DNA sequencing. In embodiments, obtaining the genetic, transcriptomic, and/or proteomic information includes epigenetic sequencing. In embodiments, obtaining the genetic, transcriptomic, and/or proteomic information includes protein sequencing.

In embodiments, for the methods provided herein, the first sequencing, second sequencing, third sequencing, and/or one or more subsequent sequencings may exclude any one of the sequencing methods recited herein. In embodiments, the first sequencing, second sequencing, third sequencing, and/or one or more subsequent sequencings is not performed using one or more of RNA-seq, DNA sequencing, epigenetic sequencing, or protein sequencing. In embodiments, the first sequencing, second sequencing, third sequencing, and/or one or more subsequent sequencings is not RNA-seq. In embodiments, the first sequencing, second sequencing, third sequencing, and/or one or more subsequent sequencings is not DNA sequencing. In embodiments, the first sequencing, second sequencing, third sequencing, and/or one or more subsequent sequencings is not epigenetic sequencing. In embodiments, the first sequencing, second sequencing, third sequencing, and/or one or more subsequent sequencings is not protein sequencing. In embodiments, the first sequencing, second sequencing, third sequencing, and one or more subsequence sequencings is not conduction DNA sequencing. In embodiments, the first sequencing is not conduction DNA sequencing. In embodiments the second sequencing is not conduction DNA sequencing. In embodiments, the third sequencing is not conduction DNA sequencing. In embodiments, the one or more subsequent sequencings is not conduction DNA sequencing.

In embodiments, for the method provided herein, the first selective pressure and/or the second selective pressure comprises treatment with a therapeutic agent, contact with a contaminant, genomic engineering, engraftment into a host, a culture condition, a growth condition, contact with a stimulus, or contact with other cells.

In an aspect is provided a method of identifying a cellular program that facilitates adaptation to a pressure. The method includes: (a) transducing cells with a plurality of barcodes such that each cell contains a single, unique barcode; (b) expanding the cells in culture to create a starting cell pool of clones of cells containing each barcode; (c) obtaining first genetic, transcriptomic, and/or proteomic information from a first subset of the starting cell pool; (d) culturing a second subset of the starting cell pool in the presence of a selective pressure to expand the starting cell pool and form an intermediate cell pool; (e) obtaining second genetic, transcriptomic, and/or proteomic information from a first subset of the intermediate cell pool; (f) continuing to culture a second subset of the intermediate cell pool in the presence of the selective pressure to expand the intermediate cell pool and form a final cell pool; (g) obtaining third genetic, transcriptomic, and/or proteomic information from at least a subset of the final cell pool; (h) quantifying a level of each barcode in the final cell pool, intermediate cell pool, and/or starting cell pool; (i) assigning cells with barcodes enriched in the final cell pool as winning clones and/or assigning cells with barcodes depleted in the final cell pool as losing clones; and (j) determining a genetic mutation, transcription program, and/or protein expression (and/or other trait) associated with at least one winning clone and/or at least one losing clone. In embodiments, the single, unique barcode is a unique combination of barcodes. In embodiments, approximately equal numbers of clones of cells containing each barcode are used to create the starting cell pool. In embodiments, the relative abundance of clones of cells are normalized relative to the numbers of cells comprising each barcode as obtained in step (c). In embodiments, steps (d) and (e) are repeated for one or more iterations thereby obtaining one or more additional intermediate cell pools and one or more additional intermediate genetic, transcriptomic, and/or proteomic information. In embodiments, step (h) further includes quantifying a level of each barcode in the one or more additional intermediate cell pools.

For the methods provided herein, in embodiments, the selective pressure includes treatment with a therapeutic agent, contact with a contaminant, genomic engineering, engraftment into a host, a culture condition, a growth condition, contact with a stimulus, or contact with other cells. In embodiments, the selective pressure includes treatment with a therapeutic agent. In embodiments, the selective pressure includes contact with a contaminant. In embodiments, the selective pressure includes genomic engineering. In embodiments, the selective pressure includes engraftment into a host. In embodiments, the selective pressure includes a culture condition. In embodiments, the selective pressure includes a growth condition. In embodiments, the selective pressure includes contact with a stimulus. In embodiments, the selective pressure includes contact with other cells.

As described above, the methods provided herein may include obtaining genetic, transcriptomic, and/or proteomic information. In embodiments, obtaining the genetic, transcriptomic, and/or proteomic information includes NGS. In embodiments, obtaining the genetic, transcriptomic, and/or proteomic information includes RNA-seq. In embodiments, the RNA-seq is single cell RNA-seq. In embodiments, obtaining the genetic, transcriptomic, and/or proteomic information comprises DNA sequencing. In embodiments, obtaining the genetic, transcriptomic, and/or proteomic information includes epigenetic sequencing. In embodiments, obtaining the genetic, transcriptomic, and/or proteomic information includes protein sequencing. In embodiments, for the methods provided herein, obtaining the genetic, transcriptomic, and/or proteomic information may exclude any one of the sequencing methods recited herein. In embodiments, obtaining the genetic, transcriptomic, and/or proteomic information does not include NGS. In embodiments, obtaining the genetic, transcriptomic, and/or proteomic information does not include RNA-seq. In embodiments, obtaining the genetic, transcriptomic, and/or proteomic information does not include DNA sequencing. In embodiments, obtaining the genetic, transcriptomic, and/or proteomic information does not include epigenetic sequencing. In embodiments, obtaining the genetic, transcriptomic, and/or proteomic information does not include protein sequencing.

### EXAMPLES

One skilled in the art would understand that descriptions of making and using the particles described herein is for the sole purpose of illustration, and that the present disclosure is not limited by this illustration.

### Example 1. Example Workflow for Longitudinal scRNA-seq Transcriptomic Analysis

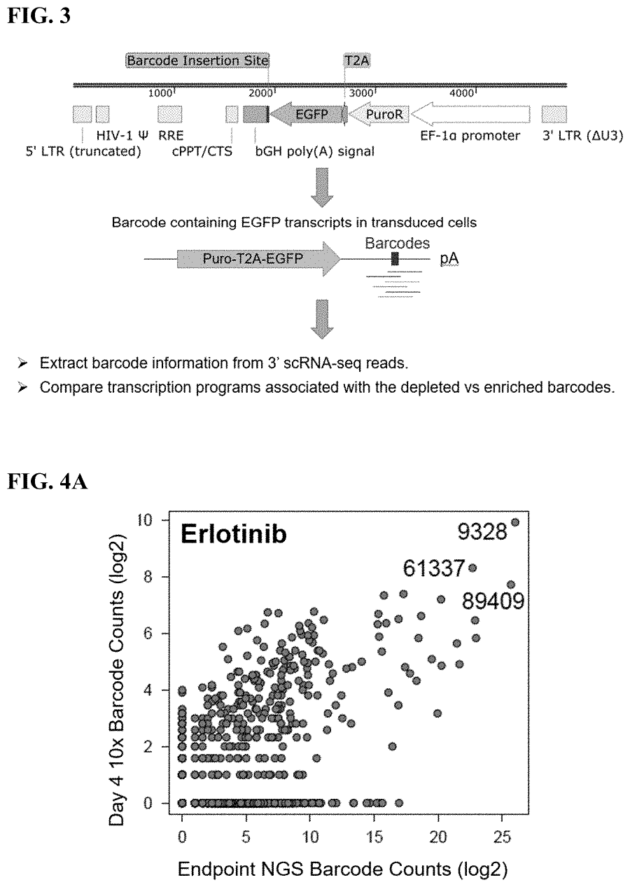

FIG. 2 shows an example workflow. A lentiviral vector, for example as shown in FIG. 3 and containing a barcode insertion site in the 3′ UTR of a Puro-IRES-GFP transgene, can be used to produce a barcoded vector library with a desired number (e.g., 1 million) different barcodes. Lentiviral particles can be produced using standard virus production protocol, and cell populations of interest can be transduced at a multiplicity of infection (MOI) of less than or equal to 0.1. This MOI was previously optimized to result in a single barcode per cell. Generally, infection of 20 million to 50 million cells results in a total of 2 million to 5 million infected cells. The infected cells can be double selected by culturing the cells in the presence of puromycin, followed by florescence-activated cell sorting (FACS) for EGFP.

To establish a barcoded pool with clones labeled by unique barcodes, the transduced cells can be dissociated to single cells and counted. N (less than or equal to 1% of the infected cells in the second step) cells can be seeded into a cell culture dish at about one cell per well and expanded to desired numbers to yield a plurality of cells composed of N single cell clones. For example, the starting cell pool may contain approximately N single cell clones that are uniquely barcoded. Greater or equal to 20N cells of the starting pool can be profiled, e.g. by single cell RNA-sequencing (scRNA-seq, such as using the 10×Technology platform).

At the same time, greater than or equal to 20N cells of the starting pool can be subjected to a selection pressure of interest, preferably with at least three technical replicates per condition. The pool of greater or equal than 20N cells can be expanded under the selection of interest and profiled at additional time points by scRNA-seq. After the selection is complete, the cells surviving to the end can be profiled again by scRNA-seq. The surviving cells may also be subjected to final barcode number quantification by genomic DNA sequencing, e.g. by next-generation sequencing (NGS) analysis. Cells with barcodes enriched at the endpoint can be assigned as “winning” clones. Cells with barcodes depleted at the endpoint can be assigned as “losing” clones. Cells with barcodes that expand the most during selection can be assigned as “super adapters.”

The transcription program associated with the winning clones, losing clones, and super adapter clones can be determined by aggregating the scRNA-seq data obtained before, during and after the selection. The transcription features associated with the winning clones before the treatment can be identified as candidate pre-existing programs that facilitate survival and adaptation with respect to the tested selection. The transcription features associated with the super adapter clones during the treatment can be identified as candidate adaptive programs that facilitate adaptation.

Further, the same starting pool can be subjected to different treatments to compare the selective pressure exerted by these treatments and/or to compare the responses and adaptive processes induced by these different treatments.

### Example 2: Identification of Pre-Existing Non-Genetic Features of EGFRi Resistant Cells

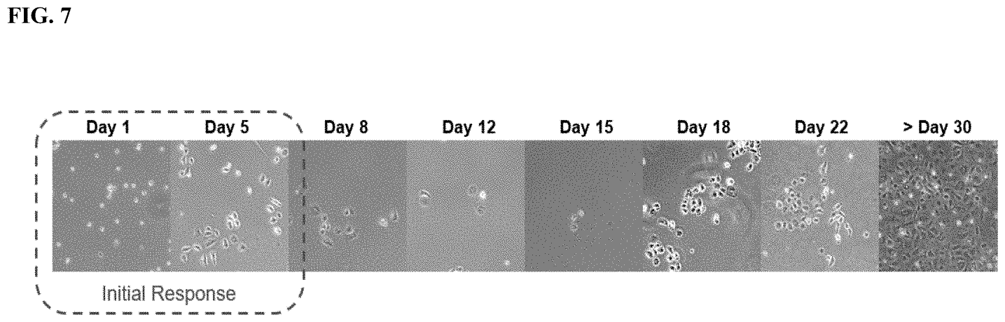

The work-flow described in Example 1 was used to identify pre-existing non-genetic features of EGFR inhibitor resistant PC9 (non-small cell lung carcinoma) cells without the requirement for purifying the resistant populations. The experimental design is shown in FIG. 6. Six hundred PC9 cells containing unique barcodes were expanded separately for 12 days. The clones were then pooled into groups of cells with replicate barcodes and subjected to a first scRNA-seq on Day 1. Subsequently, the groups of cells were treated with either DMSO (vehicle control) or Erlotinib. On Day 5 of the treatment, a second scRNA-seq was performed to evaluate an initial response of the cells to the treatments. As shown in FIG. 7, the initial response of the PC9 cells to Erlotinib is robust, with greater than 98% reduction in viability within five days compared to the DMSO control. Treatment with Erlotinib was continued for approximately two more months. At the endpoint of the treatment, a final scRNA-seq was performed on the surviving cells, which numbered greater than 4×10⁶ cells.

Pre-existing traits of cells having barcodes enriched at Day 5 were evaluated by analyzing the Day 1 sequencing data of cells having the associated barcodes as shown in FIG. 8. The pre-existing EGFR inhibitor resistant populations are known in the art, and include a genetic pre-existing T790M mutation that occurs approximately in 1 in 50,000 cells and a non-genetic metastable population that occurs approximately in 1 in 50 cells. In this experimental condition, these same non-genetic features and other genes upregulated in resistant clones prior to treatment were identified by the transcriptome data analysis from Day 1 scRNA-seq but not the much rarer genetic T790M mutation. These data indicate that pre-existing features conferring either sensitivity or resistance to different drug treatments can be rapidly identified using the methods described herein.

### Example 3: Adaptation of Lung Cancer Cells to Erlotinib or Degrader G-104

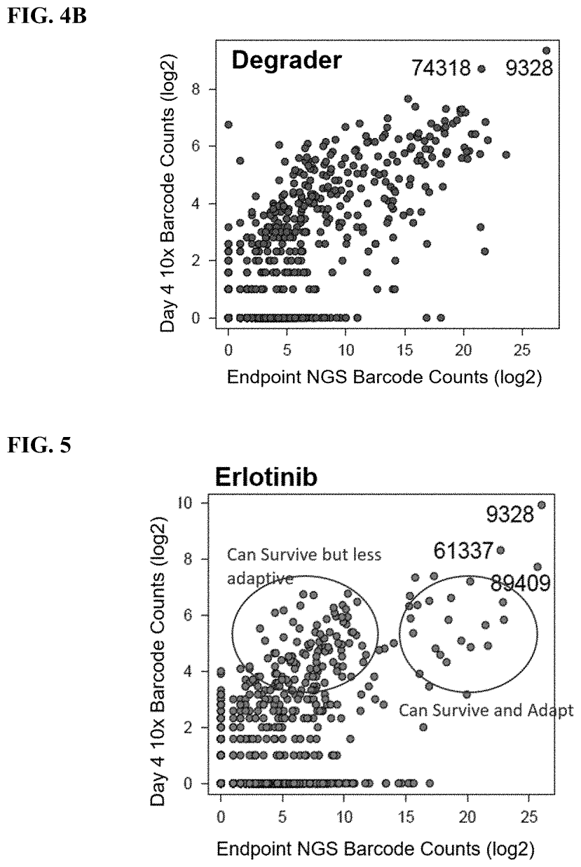

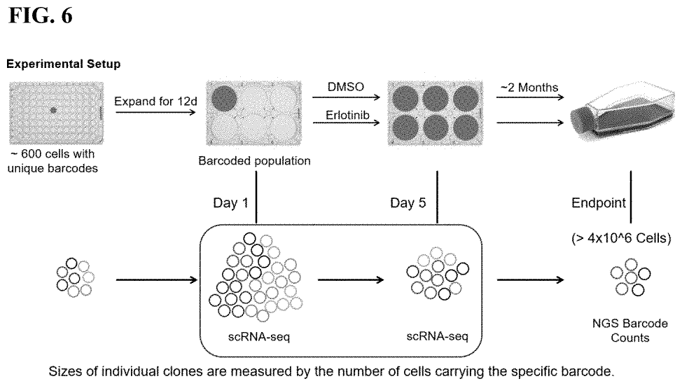

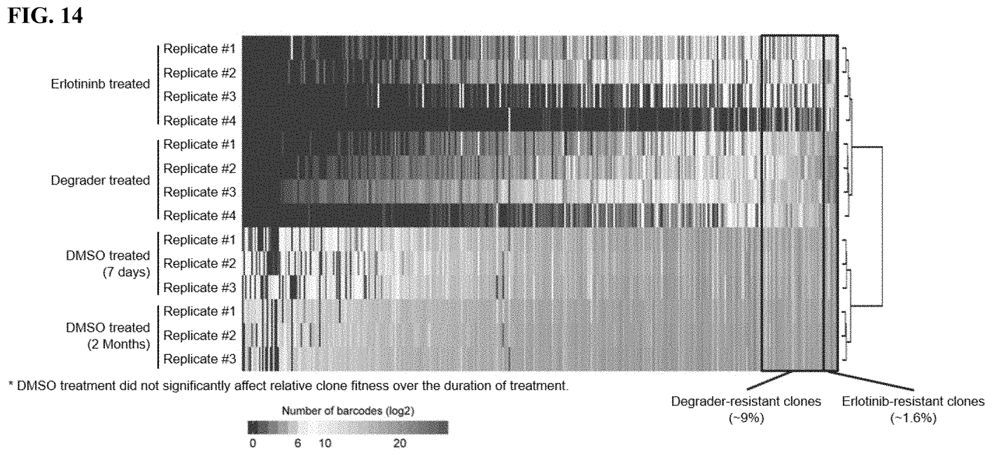

The above-described workflow was used to evaluate adaptation of mutated EGFR lung cancer cells to treatment strategies including EGFR kinase inhibition (Erlotinib) and EGFR protein degradation (Degrader G-104 or Degrader) using the PC9 (non-small cell lung carcinoma) cell line model. A representative experimental design is shown in FIG. 6. Six hundred PC9 cells containing unique barcodes were expanded separately for 12 days. The clones were then pooled into groups of cells with replicate barcodes and subjected to a first scRNA-seq on Day 1. Subsequently, the groups of cells were treated with either DMSO (vehicle control), Erlotinib, or Degrader. On Day 5 of the treatment, a second scRNA-seq was performed to evaluate an initial response of the cells to the treatments. Treatment with Erlotinib or Degrader was continued for approximately two more months. At the endpoint of the treatment, a final scRNA-seq was performed on the surviving cells. scRNA-seq data was analyzed with Next Generation Sequencing technologies, and barcode quantities were determined for both treatments, as illustrated in FIGS. 4A-4B. Analysis of barcode quantification showed that subsets of cell clones had differential survival when subjected to Erlotinib treatment compared to Degrader treatment. As illustrated in FIG. 14, a subset of barcodes were preferentially depleted by Erlotinib versus Degrader.

Pre-existing traits of cells having barcodes enriched at Day 5 were evaluated by analyzing the Day 1 sequencing data of cells having the associated barcodes. Day 1 transcriptome data showed higher expression levels of genes involved in the Epithelial Mesenchymal Transition (EMT) and EGFRi Resistance Signature for cells with barcodes enriched in both Erlotinib and Degrader treatment groups. Surviving cells in the Degrader treatment group had pre-existing low expression levels of proteasome genes and endoplasmic reticulum (ER) protein processing genes. These results indicate that cells having higher levels of expression of genes associated with migratory and invasive properties confer insensitivity to treatment with both Erlotinib and Degrader. Further, the results are consistent with knowledge in the art that treatment with Degrader induces receptor internalization, wherein cells with low expression levels of proteasome and ER protein processing genes may recruit enzymes for protein degradation to relieve ER stress, thereby bypassing cell death and adapting to Degrader.

Heat map analyses of Day 5 transcriptome data from clones having barcodes enriched at Day 5 show that compared to cells treated with Degrader, treatment with Erlotinib induced high expression levels of genes involved in the Unfolded Protein Response and ER protein processing genes in cells that were adapting at Day 5. This is consistent with knowledge in the art that subsets of cancer cells restore protein processing and relieve ER stress as a survival mechanism. In contrast, higher expression levels of the same combination of genes sensitized cells to Degrader, resulting in a reduction in barcodes associated with high levels of expression.

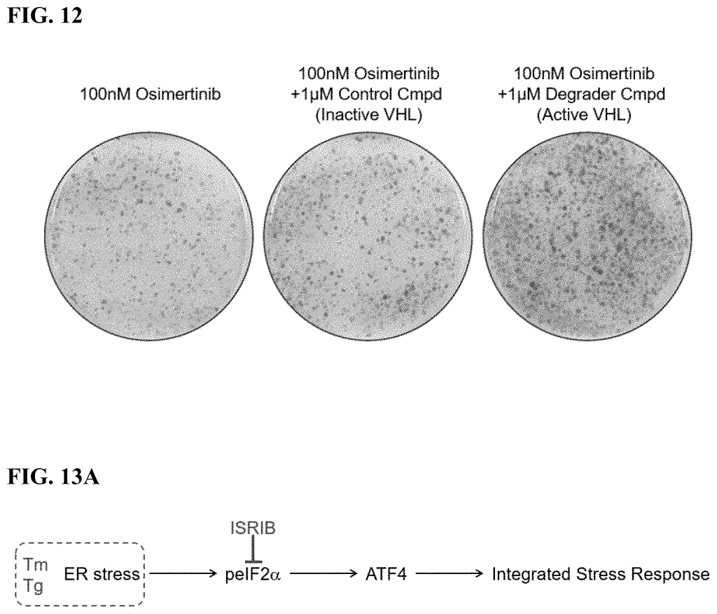

Studies with lung cancer cell lines treated with Degrader and kinase inhibitor showed that Degrader facilitates resistance development to EGFR inhibition and additionally attenuates kinase inhibitor activity. H1975 cells were treated first for 4 hours with 100 nM of the kinase inhibitor Osimertinib, then subjected to co-treatment with 100 nM Osimertinib+1 uM of Degrader for 24 hours. As illustrated in FIG. 12, addition of Degrader resulted in attenuated kinase inhibitor activity in the H1975 cells. These results indicate MOA of conventional kinase inhibitors and Degraders vary in mediating therapeutic efficacy and confirm differential survival of clonal populations treated with Degrader verses Erlotinib.

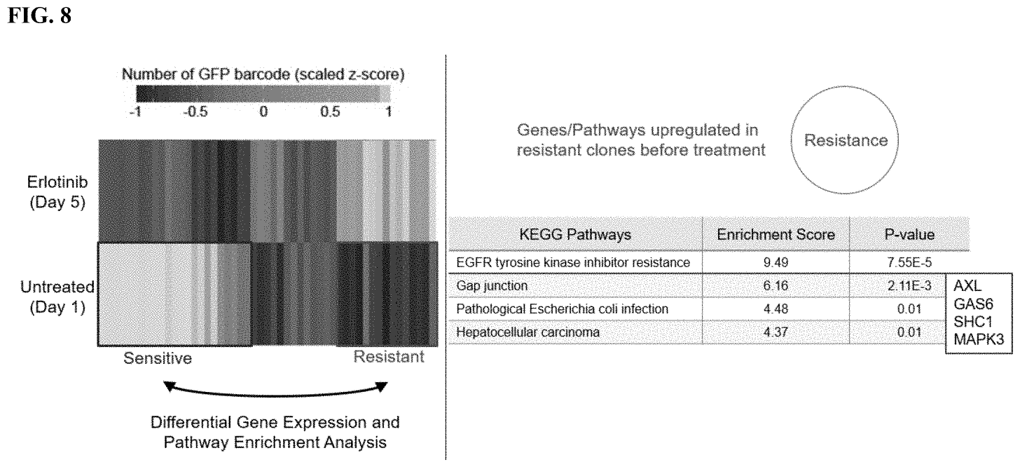

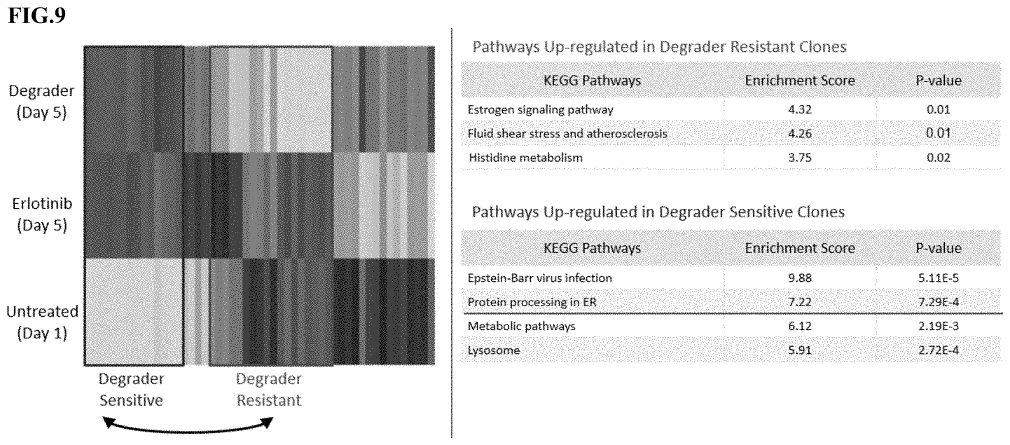

Using an experimental set-up similar to that shown in FIG. 6, Degrader resistant and sensitive clonal populations were identified. FIG. 9 illustrates heat map analysis of Day 5 transcriptome data from clones having depleted or enriched barcodes. Analysis of single cell RNA-seq transcriptome data allowed identification of specific pathways upregulated in Degrader resistant clones, and confirmed that Degrader resistance is driven by certain mechanisms distinct from those known to confer resistance to EGFR kinase inhibitors.

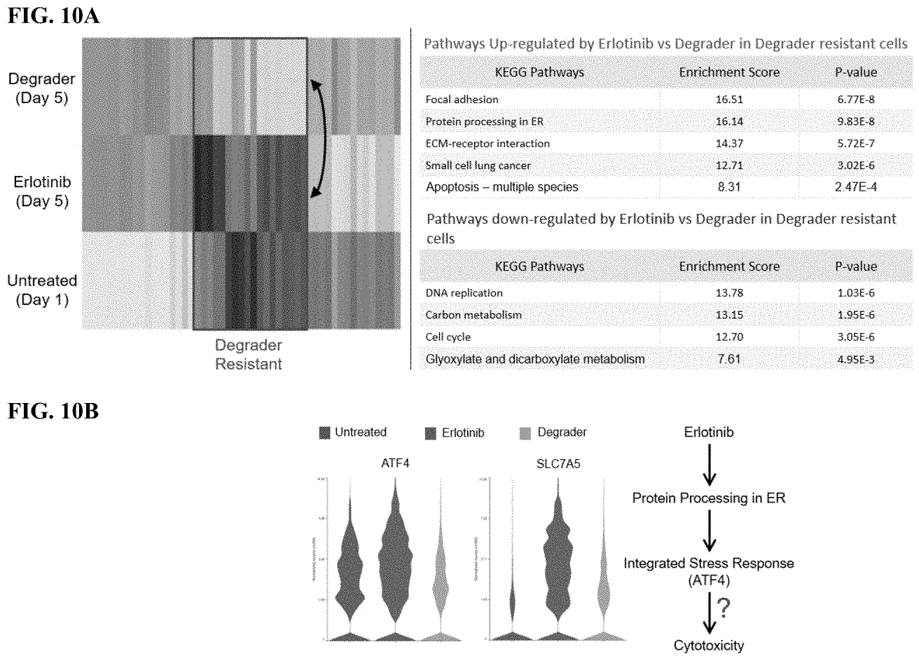

Single cell RNA-seq data was further analyzed to evaluate how Degrader resistant clones respond differently to Erlotinib versus Degrader. FIG. 10A shows heat map analysis of Day 5 transcriptome data comparing Erlotinib affected pathways to Degrader affected pathways in the Degrader resistant clones. Compared to Degrader, Erlotinib activated genes involved in ER protein processing pathways. Expression of genes ATF4 and SLC7A5, which are associated with ER stress-mediated cell death and lead to Integrated Stress Response (ISR) induction, were additionally upregulated on Day 5 of Erlotinib treatment, as shown in FIG. 10B. These results indicate Erlotinib may function as a cytotoxic agent by activating the ER protein processing and ISR pathways. Further, Erlotinib was shown to down-regulate pathways including DNA replication, carbon metabolism and cell cycle, which are consistent with and reflect growth arrest induced by Erlotinib.

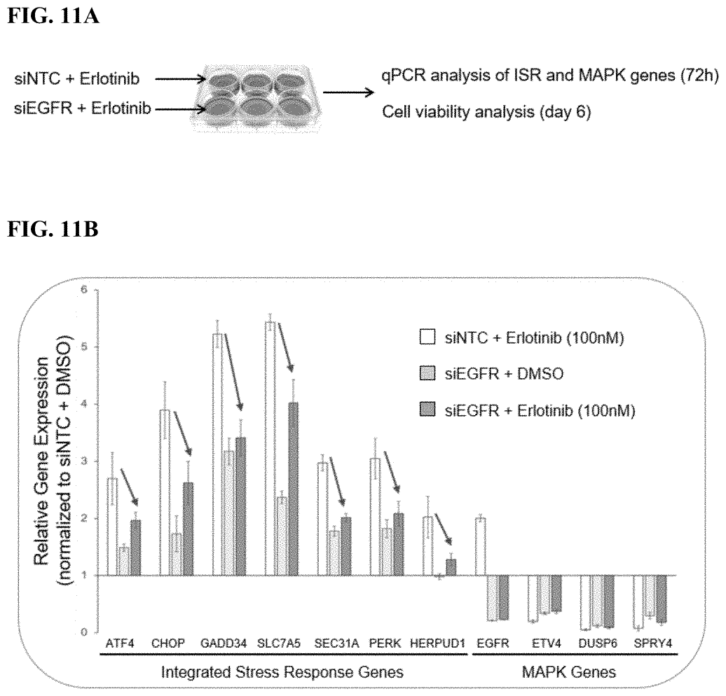

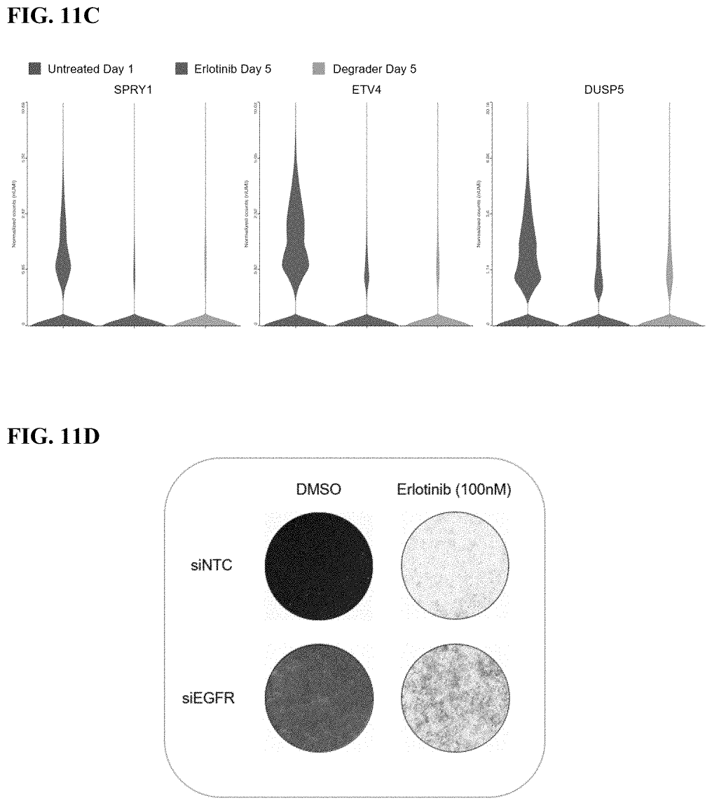

Since the ISR pathway was shown to be activated by treatment with Erlotinib, but not by Degrader, depletion of EGFR protein production was investigated to model protein degradation. Thus, PC9 cells were treated with either siNTC+Erlotinib, siEGFR+Erlotinib, or siEGFR+DMSO, then analyzed for ISR and MAPK genes after 72 hrs (via scRNA-seq), and assessed for cell viability at Day 6. The experimental setup is shown in FIG. 11A. Results illustrated in FIG. 11B show that acute depletion of newly synthesized EGFR protein causes downregulation of gene expression associated with ER stress and ISR pathway induction. MAPK gene expression was used as a control, as both Erlotinib and Degrader strongly suppressed canonical MAPK target genes, as shown in FIG. 11C. Results shown in FIG. 11D illustrate decreased efficacy of Erlotinib when combined with siEGFR, as indicated by greater numbers of viable cells in the Erlotinib+siEGFR treatment group compared to treatment by only Erlotinib. Collectively, these results indicate inhibition of EGFR protein production by Degrader attenuates ISR induction and efficacy of EGFR inhibitors.

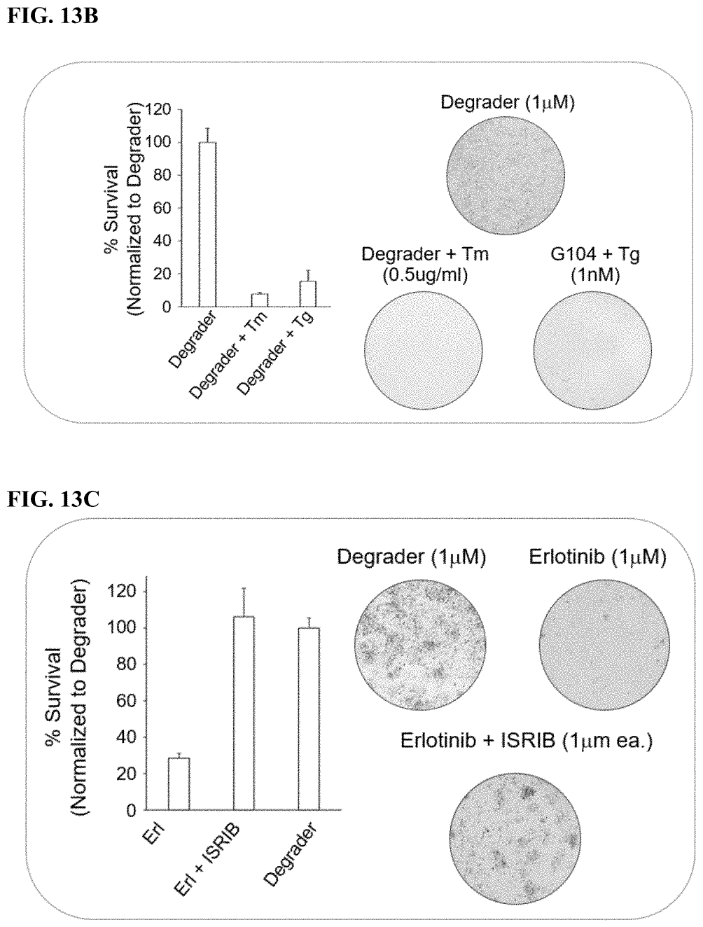

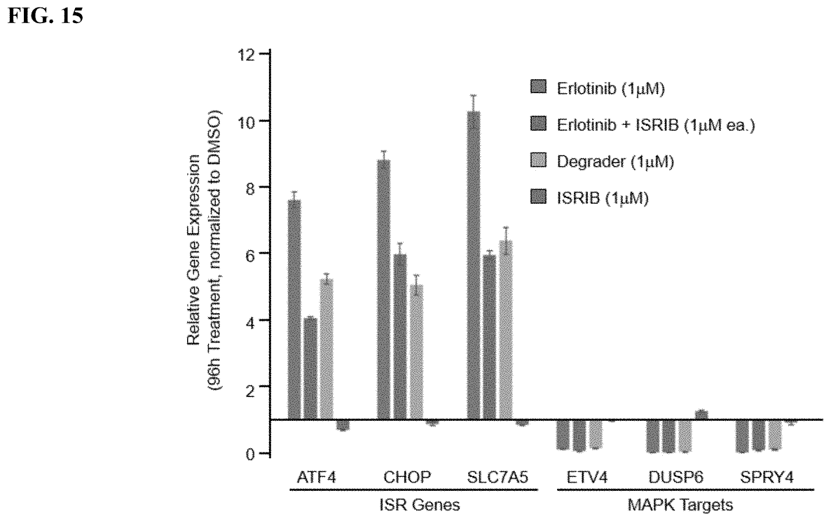

Pharmacological modulations of the ISR were tested alongside Degrader or Erlotinib, to further investigate MOAs as revealed by the scRNA-seq transcriptome data. FIG. 13A shows a schematic of proteins involved in ISR induction and pharmacological modulations of the proteins. Tunicamycin and Thapsigargin were used as inducers of ER stress, and Integrated Stress Response Inhibitor (ISRIB) was used to inhibit ISR activation. Cells were treated with Degrader, Degrader+Tunicamycin, or Degrader+Thapsigargin to assess the effect of ER stress on Degrader efficacy. In another experimental group, cells were treated either with Degrader, Erlotinib, or Erlotinib and ISRIB. Results illustrated in FIG. 13B show that the combination of Degrader with induction of mild ER stress results in enhanced efficacy of Degrader. This confirms results from scRNA-seq data analysis that Degrader by itself does not activate the ISR pathway. As shown in FIG. 13C, addition of ISRIB to Erlotinib results in loss of efficacy of Erlotinib, demonstrated by increase in cell viability. Inhibition of Erlotinib ISR gene activation by ISRIB was further confirmed by qPCR analysis, as shown in FIG. 15.

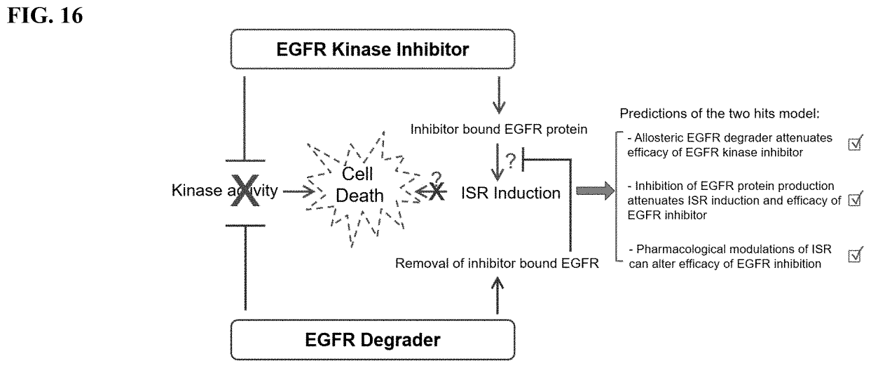

The above-described experiments underscore the power of tracking barcoded cells using the scRNA-seq platform to reveal both drug MOAs and resistance mechanisms. Using the methods described herein, EGFR kinase inhibitors were shown to function by both inhibiting EGFR and activating the ISR pathway, thereby exerting cytotoxic effects with a two-prong pathway, as shown in FIG. 16.

These data show that the responses of a clonal population over time to treatment of interest can be tracked, and pre-existing features that confer sensitivity vs insensitivity, programs that facilitate or prevent adaptations, as well as eventual adaptive states and outcomes can be connected. This can be done at single cell resolution. At the same time, this technology also takes advantage of high-throughput technologies, such that transcription programs can be more accurately captured by aggregating scRNA-seq data collected from cells within the same clone and/or clones with similar adaptive trajectories. In contrast, previous models only compare the starting population with the population after selection, thus failing to resolve pre-existing traits vs adaptive changes.

### Example 4: Identification of Resistance Mechanisms of KRAS Cancers and Development of Combination Therapeutics

Although the G12C mutation in KRAS is known to play a crucial role in aggressive cancers, therapeutics to inhibit the mutant KRAS have proved challenging. The above-described workflow will be applied to reveal resistance mechanisms to mutant KRAS inhibitors and inform development of protocols for administration of KRAS inhibitors in combination with other therapeutic agents. The major KRAS G12C mutant inhibitor resistance mechanisms in preclinical models include non-genetic features and intrinsic and/or inherent properties that determine KRAS dependency. The above-described methods will be used to address how resistant cells respond differently to KRAS inhibitors compared to sensitive cells in a heterogeneous population, and will help identify which genes and/or pathways enable initial cell survival. Further, the methods will assist in identifying mechanisms surviving cells use to adapt to KRAS inhibition and become fully resistant. Collectively, these results will assist in determining which drug combinations are most effective in eliminating the pre-existing resistant cells.

### Example 5: TraCe-Seq Reveals Pre-Existing and Adaptive Features that Underlie the Unexpected Inferior Efficacy of Targeted EGFR Degradation Compared to Inhibition

Genetic and non-genetic heterogeneity within cancer cell populations represents a major challenge to anti-cancer therapies. There is a current lack of robust paradigms that address how pre-existing and adaptive features impact cellular responses to therapies. Here, a method has been developed, TraCe-seq, by combining clonal tracking and single-cell RNA-sequencing to capture the origin, fate, and differential early adaptive programs of cells within a complex population in response to distinct treatments at clonal resolution.

TraCe-seq was used to benchmark how next-generation dual EGFR inhibitors-degraders compare to standard EGFR kinase inhibitors in EGFR-mutant lung cancer cells. A paradoxical loss of anti-growth activity associated with targeted degradation of EGFR protein and an unexpected and essential role of the ER protein processing pathway in anti-EGFR therapeutic efficacy were identified. This example study challenges the assumption that targeted degradation would be universally superior to enzymatic inhibition, and demonstrates TraCe-seq as a broadly applicable approach to study how pre-existing transcriptional programs impact treatment response.

Targeted therapies against oncogenic driver mutations have provided significant clinical benefit to cancer patients and hold great promise for precision medicine. However, not all patients harboring such mutations respond equally. While resistance mechanisms like secondary-site alterations have been reported, other pre-existing and acquired resistance-conferring mechanisms pose a great challenge to the overall response and durability in the clinic even among the best-studied therapies.

Anti-cancer therapeutic options have continued to evolve to enhance response and overcome resistance. While direct inhibition of the oncogenic driver (perhaps as best exemplified by the success of small molecule inhibitors of kinases) remains an integral part of precision therapy, new therapy modalities are also being explored. Recently, there is growing interest in a novel mechanism of action (MOA), that of targeted protein degradation, over conventional occupancy-based target inhibition. Heterobifunctional targeted protein degraders, molecules that can simultaneously recruit an E3 ubiquitin ligase to the target of interest and induce targeted degradation through ubiquitin-mediated proteolysis, have gained tremendous interest and have been shown to be superior compared to enzymatic inhibition alone in specific contexts. However, it remains unknown whether dual action inhibitor/degraders will present a universal advantage.

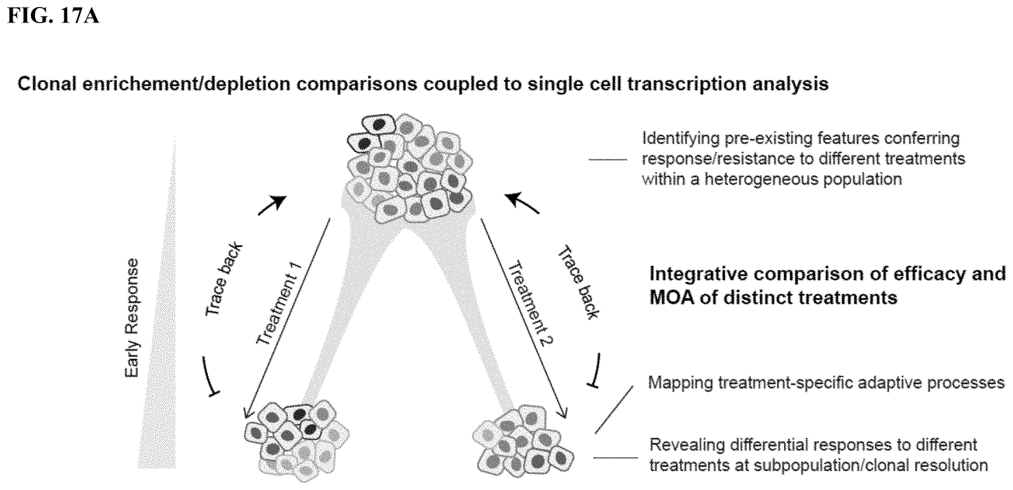

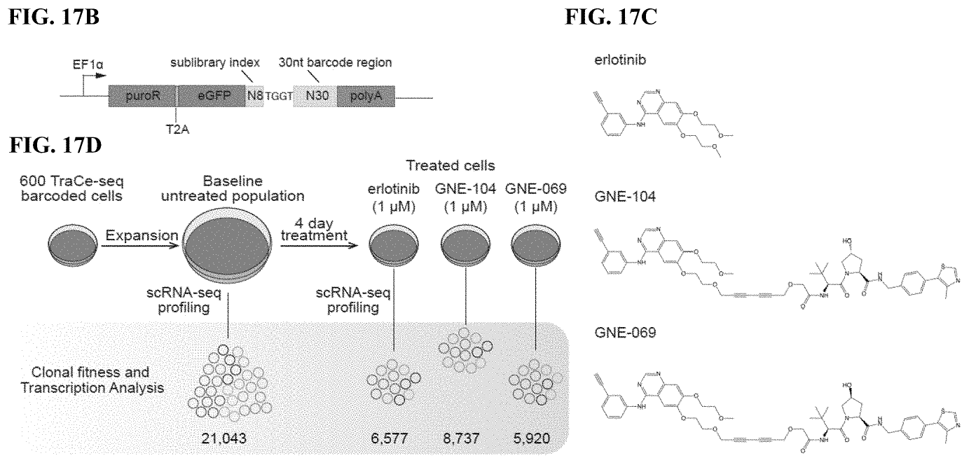

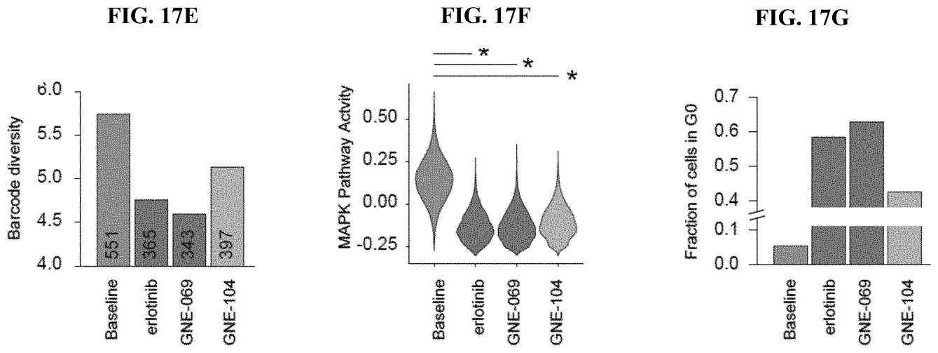

Current paradigms to characterize drug response and resistance depend heavily on endpoint assessment after prolonged drug exposure. While this process can be effective to identify mechanisms of resistance, it is limited in its ability to uncover pre-existing features and early adaptive changes that enable specific subpopulations to persist. There is a need for a system that simultaneously tracks the origin and compares the immediate responses/fate of tumor cells to different therapies could greatly accelerate drug response/resistance studies. This would allow direct comparisons of efficacy and MOAs of different therapies, thus further accelerating development of future treatments. To this end, the method TraCe-seq (Tracking Differential Clonal Response by scRNA-sequencing) was developed to rapidly capture the origins and early adaptive processes underlying therapy response by simultaneously measuring clonal fitness and their transcriptional trajectories in a heterogeneous population subject to anti-cancer treatments (FIG. 17A). As demonstrated below, this method enables direct and comprehensive comparisons of different therapeutic modalities at subpopulation and single cell resolution and provides novel insights into pre-existing transcriptional features that dictate drug response and subsequent resistance.

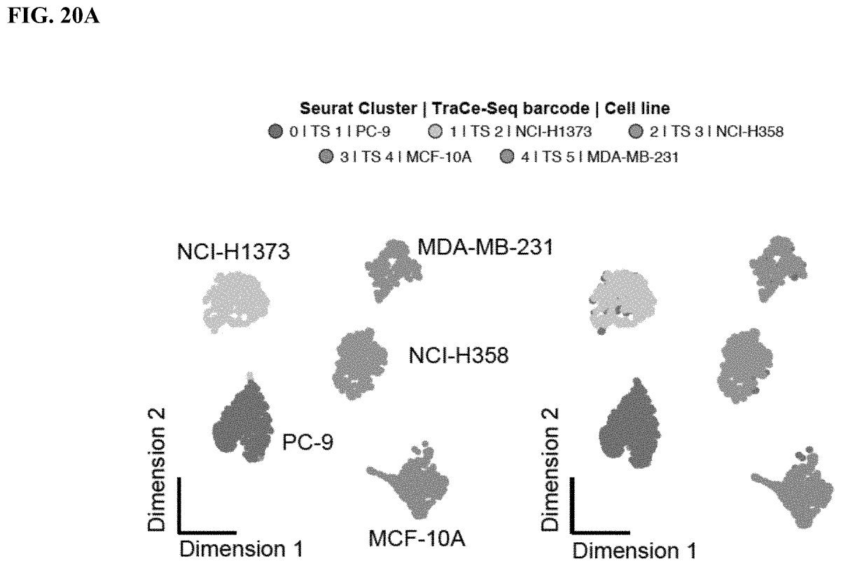

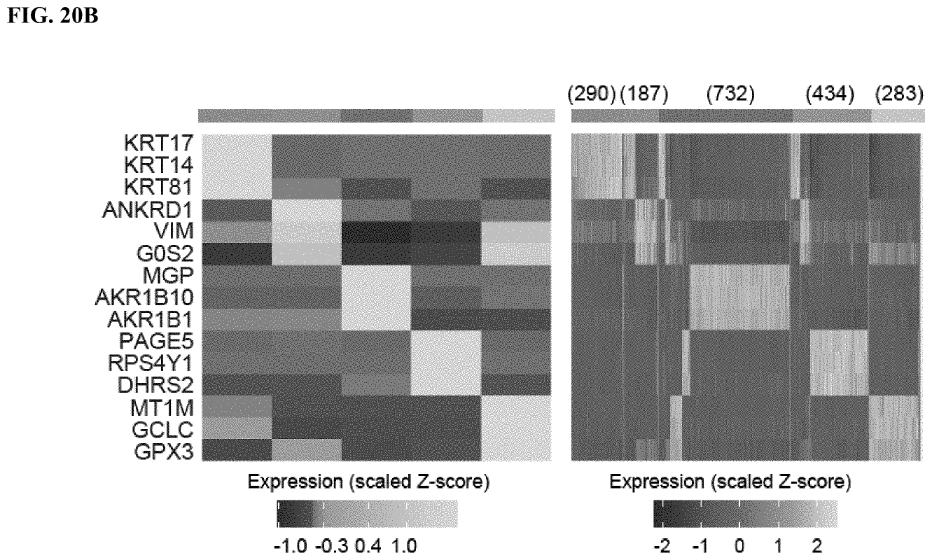

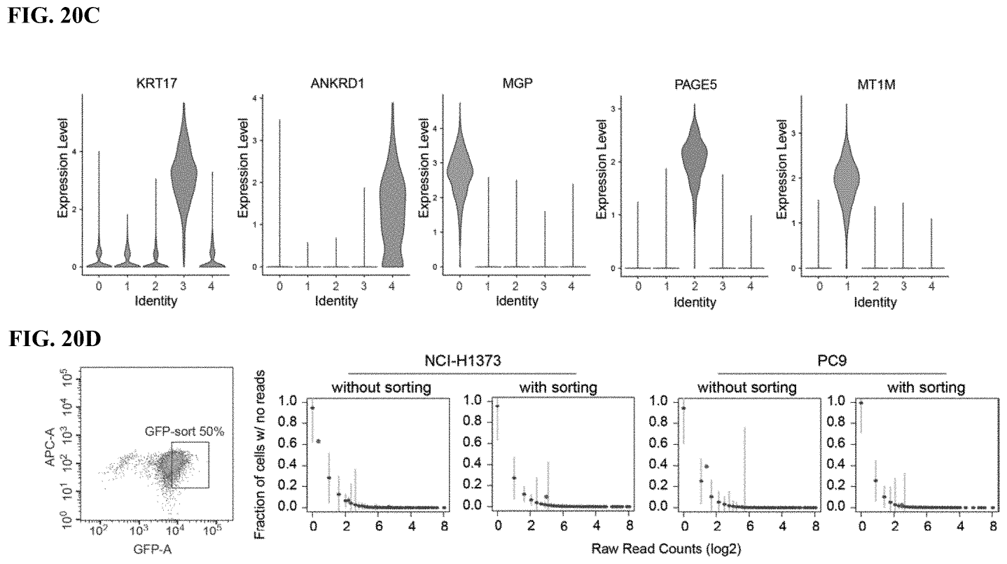

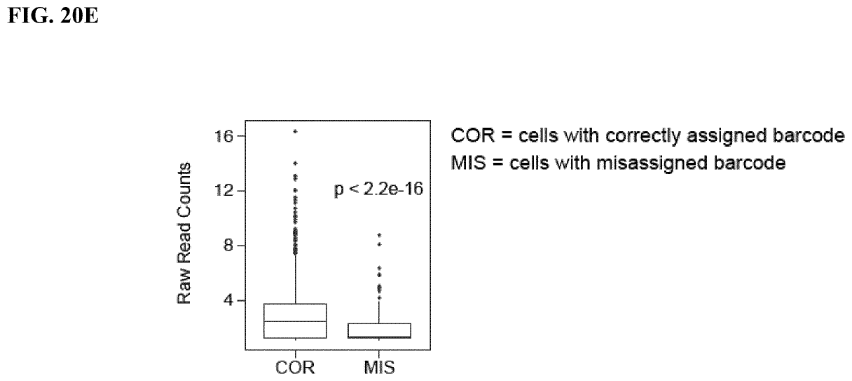

To set up TraCe-seq, a 3′ scRNA-seq compatible lentivirus based barcoding library was constructed, similar to recent reports. Each barcode is composed of a 30-nt region with optimal GC content at 100,000×diversity. Also incorporated an 8-nt sub-library index to allow flexible control of total library size. Upon successful transduction, the lentiviral vector will be stably integrated into the genome, resulting in constitutive expression of selection markers, puromycin-resistance and eGFP, and a barcode embedded in the 3′-untranslated regions of the reporter cassette (FIG. 17B). To confirm our ability to recover these transduced barcodes, to ensure the system can differentiate underlying biology, and to assess whether dual puromycin and eGFP selection is needed, a unique barcode was transduced into 5 different cell lines (PC9, MCF-10A, MDA-MB-231, NCI-H358, and NCI-H1373). Transduced cells were selected with puromycin only, mixed together, and the complex mixture of the 5 cell lines was profiled by 10× scRNA-seq. Individual cell expression profiles were clustered and projected in a two-dimensional space using UMAP. TraCe-seq barcodes were independently mapped and annotated on a per-cell basis. Unsupervised clustering of the five cell lines resulted in five distinct transcriptomic clusters, and 81% of TraCe-seq barcodes were correctly assigned to the correct transcriptomic cluster (FIG. 20A). Marker genes from an unsupervised scRNA-seq differential gene expression analysis of each TraCe-seq barcode population were in line with gene expression using bulk RNA-seq of respective cell lines (FIGS. 20B, 20C). Overall, barcode detection and mapping varied as a function of its expression (FIGS. 20D, 20E). Cells with lower average barcode expression had higher barcode dropout rate, which followed the same dropout pattern as other endogenously expressed genes (FIG. 20D). To overcome this limitation, fluorescence-activated cell sorting (FACS) for the top 50% eGFP-expressing cells greatly enhanced barcode expression and boosted barcode recovery rate to above 90% (FIG. 20D). Taken together, the dual selection marker system of the TraCe-seq vector is necessary to ensure optimal barcode detection and assignments.

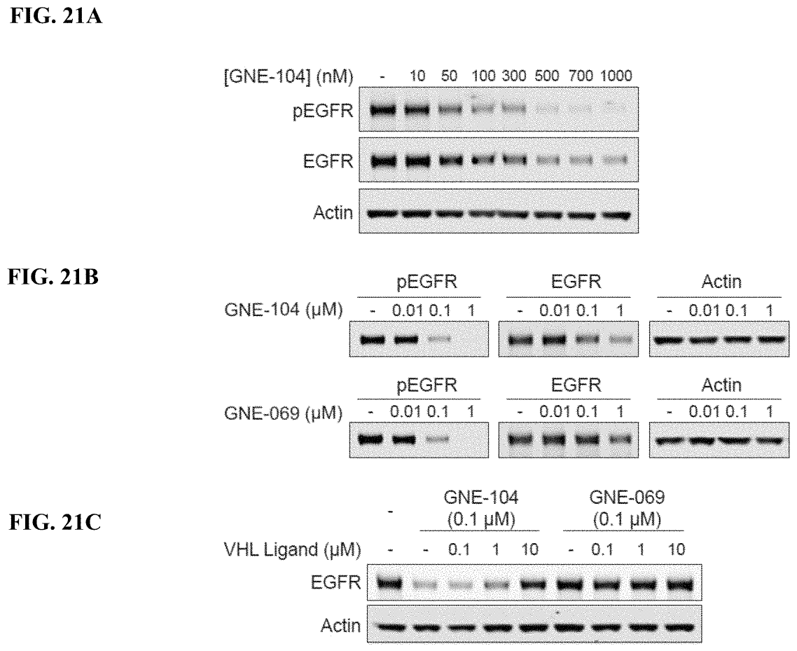

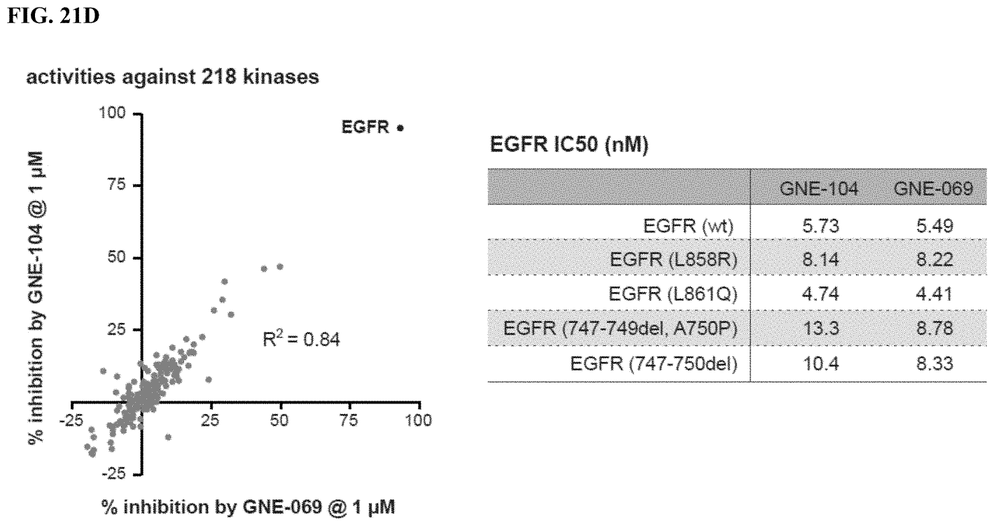

As EGFR-directed therapies are a mainstay of precision oncology therapy, TraCe-seq was used to better understand response to EGFR therapies with unique MOAs. Despite initial therapeutic success, most patients harboring common EGFR inhibitor sensitizing mutations (L858R or Exon19 deletions) ultimately develop resistance to EGFR-directed therapies, with the majority of patients lacking additional secondary-site EGFR mutations when treated with the current standard-of-care EGFR inhibitor osimertinib. While small molecule strategies to target EGFR have focused on inhibiting its enzymatic activity, little is known of the potential outcome of EGFR degradation in contrast to enzymatic inhibition. To this end, GNE-104 was developed, a heterobifunctional degrader composed of the EGFR inhibitor erlotinib linked to a VHL binding moiety, the substrate-binding component of the CRL4-VHL E3 ligase complex. GNE-104 induced significant dose-dependent degradation of EGFR as well as potently suppressed phospho-EGFR levels (FIG. 17C and FIG. 21A). A non-degrader control GNE-069 was generated (FIG. 17C), which fails to engage VHL (due the specific stereochemistry in the VHL-binding moiety) and degrade EGFR (FIGS. 21B, 21C), whilst retaining a comparable potency and selectivity profile as GNE-104 in in vitro kinase inhibition assays (FIG. 21D).

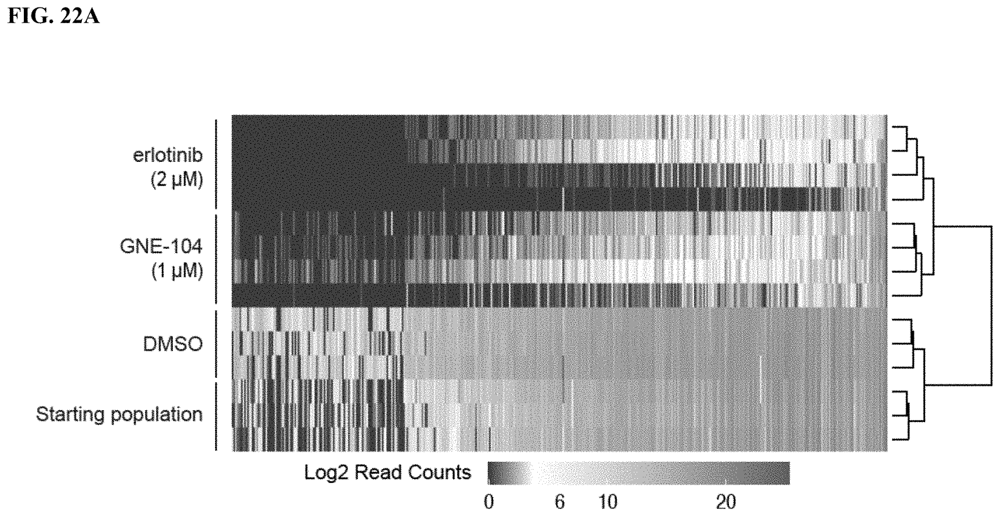

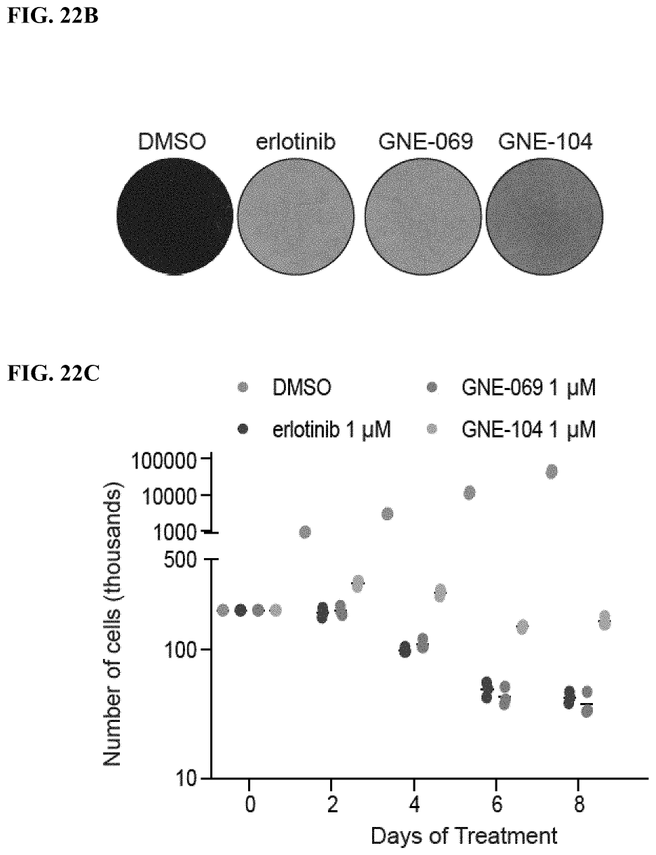

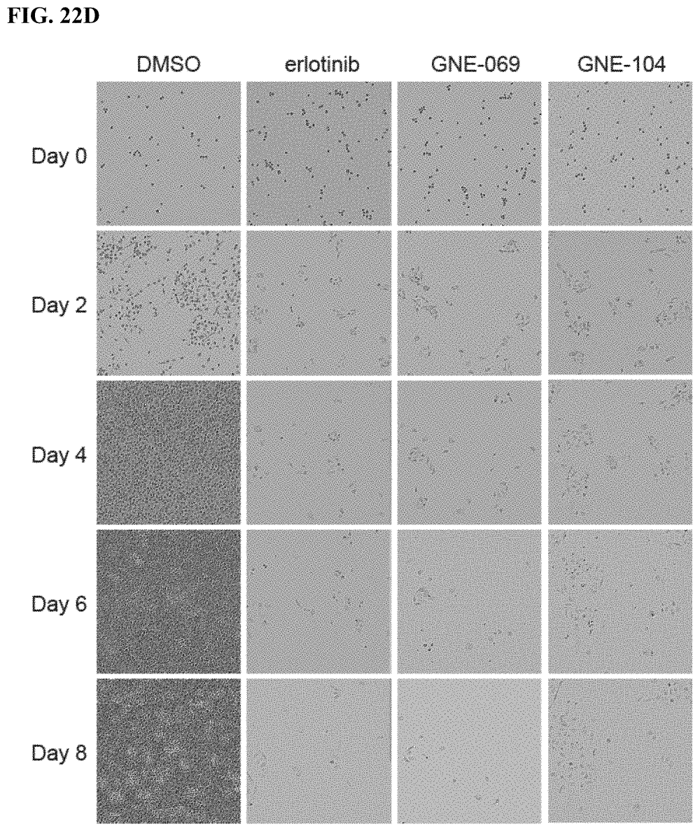

Next, we proceeded to establish whether TraCe-seq can: (1) distinguish drug sensitive versus resistant clones upon treatment with a specific molecule; (2) capture known genes associated with EGFR inhibitor resistance; and (3) differentiate response and resistance mechanisms of EGFR degraders versus conventional EGFR kinase inhibitors. PC9 cells were chosen as a model system as they responds robustly to EGFR inhibition and contain a well-documented subpopulation of resistant cells that is driven by non-genetic mechanisms. To determine whether it was possible to effectively and reproducibly capture the spectrum of EGFR inhibitor resistance/sensitivity phenotype in PC9 cells, we first conducted a pilot study on 500 TraCe-seq barcoded clones. These clones were expanded for 12 doublings and treated 15,000 cells from the resulting population with erlotinib or GNE-104 for two months in replicates and determined barcode enrichment patterns from genomic DNA of the surviving populations. Overall, clonal survival was highly consistent under individual treatment condition and differed between erlotinib and GNE-104 treatment (FIG. 22A). Thus, it was concluded that this system faithfully captures the spectrum of response and resistance to EGFR inhibition in PC9 cells under similar conditions.

To determine the suitable time point for tracking and comparing immediate responses to EGFR kinase inhibitor versus degrader, the kinetics of the treatment response were characterized over eight days. Paradoxically and intriguingly, despite promoting both kinase inhibition and EGFR degradation, it was observed that GNE-104 was far less efficient in inhibiting cell growth compared to erlotinib and GNE-069 using multiple assay formats (FIGS. 22B-22D). Based on the kinetics of the response to the drugs, it was decided to sample the population at day 4, when significant growth inhibition had occurred but before sensitive cells had fully vanished (FIG. 22C), so that it could be captured and the transcriptional responses of the drug sensitive versus the drug resistant populations could be compared.

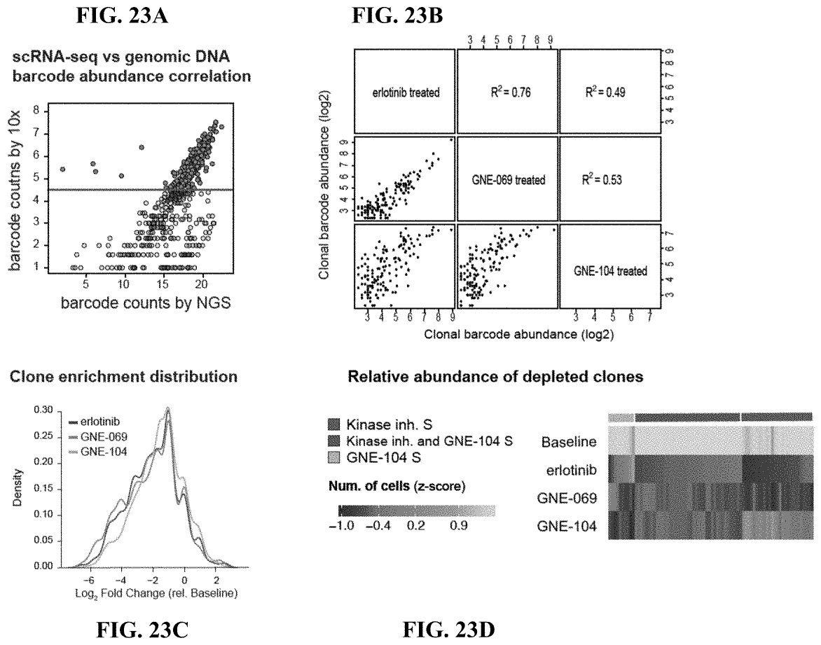

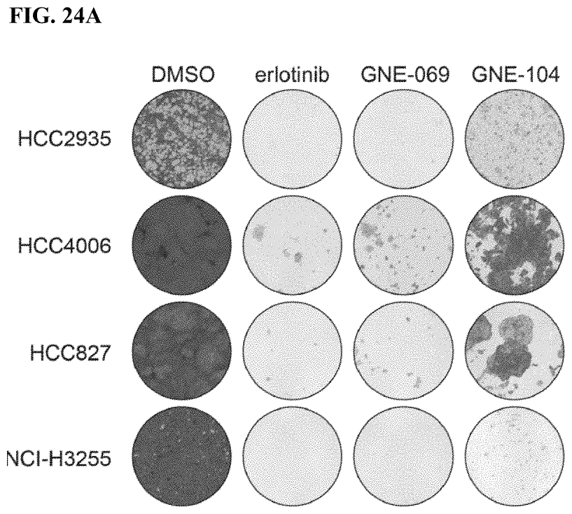

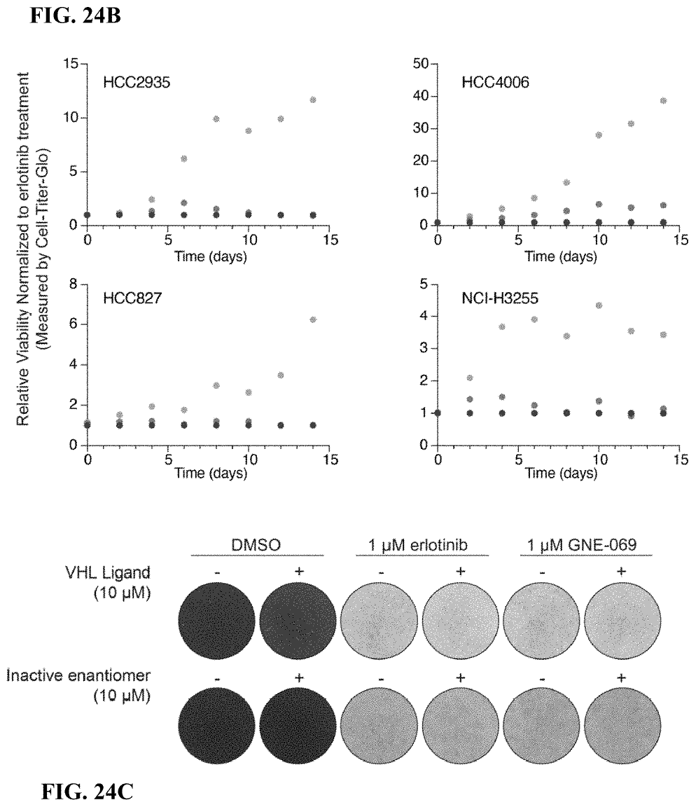

To conduct the TraCe-seq experiment, 600 PC9 cells carrying unique barcodes were randomly selected and these cells were expanded for ˜12 doublings to establish the starting population. Single cell transcriptional profiling on a fraction of this population at ˜30× coverage of the barcodes was performed to record the baseline transcriptional programs and relative clonal abundance. 200K of these cells were seeded and were treated with either erlotinib, GNE-104, or GNE-069. Cells were harvested on day four and profiled by 10×scRNA-seq to determine the relative fitness of each clone and capture treatment-induced transcriptional changes (FIG. 17D). To confirm that the TraCe-seq approach accurately measures relative clonal sizes at baseline (inferred from TraCe-seq barcode abundance), 20K barcoded cells were expanded in DMSO for seven days and profiled these cells by saturating barcode analysis from genomic DNA (>3000×coverage). Indeed, clonal abundance determined by scRNA-seq versus genomic DNA analysis were highly correlated (FIG. 23A). 551 barcodes at baseline (FIG. 17E) prior to treatment were recovered. All treatment conditions resulted in decreased barcode diversity (−16.0±5%). Relative clonal abundance was highly similar under erlotinib and GNE-069 treatment, reflecting their highly comparable activities (and moving forward will be grouped together under the term kinase inhibitor), whereas GNE-104 treatment resulted in a more divergent pattern (FIG. 23B). Consistent with the observed reduced growth inhibitory activity of GNE-104 (FIGS. 22B-22D), GNE-104 treatment was less effective in reducing both the absolute number and the diversity of TraCe-seq barcodes (FIG. 17E). Furthermore, although comparable MAPK pathway suppression was observed across all treatment conditions (FIG. 17F), erlotinib and GNE-069 induced far greater cell cycle arrest (GO) than GNE-104 (p<10¹⁴⁵) (FIG. 17G), suggesting that GNE-104 treatment allowed for more cells to persist in the presence of MAPK pathway inhibition. The reduced anti-growth effect of GNE-104 was confirmed in multiple EGFR-mutant lung cancer cell lines (FIGS. 24A and 24B). This loss of efficacy was not due to the presence of the active VHL ligand, as ten times higher concentration of free VHL ligand did not affect the response to erlotinib or GNE-069 (FIG. 24C).

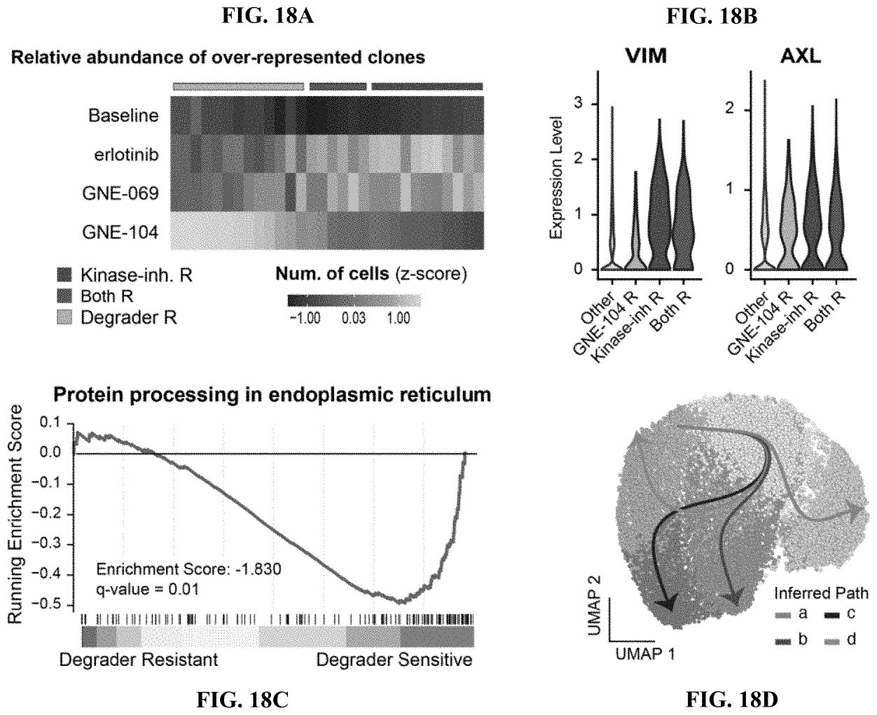

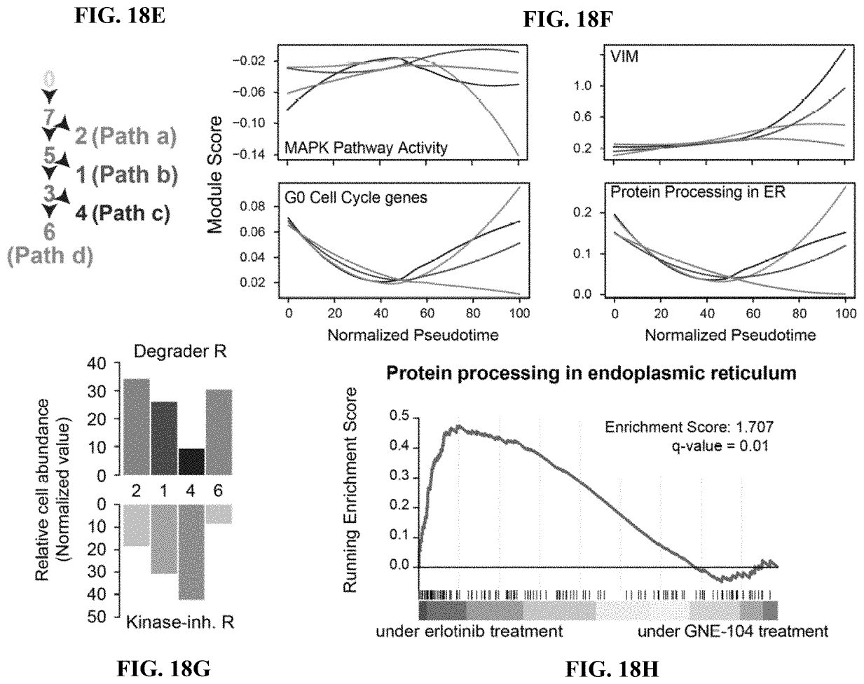

To further understand the differential response of PC9 cells to these treatments at the clonal level, it was sought to categorize Trace-seq barcodes that were over- or underrepresented in the treated population compared to the starting population (FIG. 23C). TraCe-seq barcodes that were overrepresented under treatments compared to the untreated population were categorized into three classes of resistant clones (FIG. 18A), while barcodes that were underrepresented under treatments were categorized into three classes of sensitive clones based on their responses to kinase inhibitors versus degraders (FIG. 23D). In particular, GNE-104 treatment resulted in overrepresentation of a unique set of Trace-seq barcodes compared to kinase inhibitors that were categorized as degrader resistant (FIG. 18A). It was hypothesized that over- and underrepresented clones possessed differential baseline features that commit cells to respond or persist under subsequent treatments. To explore this, it was sought to identify gene expression differences between the drug-sensitive versus drug-resistant clones at baseline under the untreated condition. Unbiased differential gene expression analysis was performed to look for pre-existing transcriptional features that may confer kinase-inhibitor (erlotinib and GNE-069) resistance by comparing baseline gene expression between kinase-inhibitor resistant versus kinase-inhibitor sensitive clones. Consistent with previous reports, it was found that VIM and AXL were significantly upregulated in the kinase-inhibitor resistant cells prior to treatment (FIG. 18B), confirming that TraCe-seq successfully captured the expected resistant subpopulation and associated molecular markers. Importantly, it was found that degrader-resistant clones only displayed elevated AXL expression, but showed no upregulation of VIM, suggesting that they indeed differed transcriptionally from kinase inhibitor resistant clones (FIG. 18B). To uncover what transcriptional programs may enable survival of cells under degrader treatment, unbiased gene set expression analyses were performed comparing degrader-resistant clones versus degrader sensitive clones (Table 1). Intriguingly, “protein processing in ER” activity was found to be the most significant and most enriched pathway that differed between degrader-resistant clones from degrader sensitive clones (FIG. 18C), suggesting that cells with lower expression of ER protein processing genes may be protected from GNE-104.

TABLE 1

| | | | Enrichment | | |
|---|---|---|---|---|---|
| ID | Description | setSize | Score | NES | qvalues |
| hsa04141 | Protein processing in | 138 | −0.49301 | −1.84556 | 0.01431 |
| | endoplasmic reticulum | | | | |
| hsa05170 | Human immunodeficiency virus 1 | 146 | −0.46475 | −1.75633 | 0.01431 |
| | infection | | | | |
| hsa05169 | Epstein-Barr virus infection | 126 | −0.41751 | −1.55222 | 0.02807 |
| hsa05165 | Human papillomavirus infection | 205 | −0.37507 | −1.46386 | 0.02807 |
| hsa05163 | Human cytomegalovirus infection | 143 | −0.37616 | −1.41159 | 0.05127 |
| hsa04810 | Regulation of actin cytoskeleton | 128 | −0.33162 | −1.23338 | 0.14735 |
| hsa04144 | Endocytosis | 184 | −0.32130 | −1.23313 | 0.11786 |
| hsa05168 | Herpes simplex virus 1 infection | 201 | −0.31448 | −1.22139 | 0.11786 |
| hsa05166 | Human T-cell leukemia virus 1 | 149 | −0.31046 | −1.17647 | 0.14875 |
| | infection | | | | |
| hsa04218 | Cellular senescence | 123 | −0.31551 | −1.16577 | 0.17887 |
| hsa05203 | Viral carcinogenesis | 131 | −0.27862 | −1.04160 | 0.32119 |
| hsa05131 | Shigellosis | 161 | 0.24781 | 1.01503 | 0.33097 |
| hsa05010 | Alzheimer disease | 213 | 0.27092 | 1.14389 | 0.14875 |
| hsa05205 | Proteoglycans in cancer | 128 | 0.29407 | 1.16053 | 0.17887 |
| hsa04151 | PI3K-Akt signaling pathway | 175 | 0.28762 | 1.18789 | 0.14735 |
| hsa04714 | Thermogenesis | 155 | 0.29346 | 1.19684 | 0.14818 |
| hsa05016 | Huntington disease | 182 | 0.31100 | 1.29674 | 0.09092 |
| hsa05130 | Pathogenic Escherichia coli | 130 | 0.33522 | 1.32952 | 0.10588 |
| | infection | | | | |
| hsa05132 | Salmonella infection | 156 | 0.33116 | 1.35115 | 0.08214 |
| hsa03010 | Ribosome | 128 | 0.36210 | 1.42903 | 0.05127 |
| hsa05012 | Parkinson disease | 175 | 0.34665 | 1.43170 | 0.04819 |
| hsa03013 | RNA transport | 136 | 0.37526 | 1.48667 | 0.02820 |
| hsa03040 | Spliceosome | 124 | 0.52705 | 2.05634 | 0.01869 |

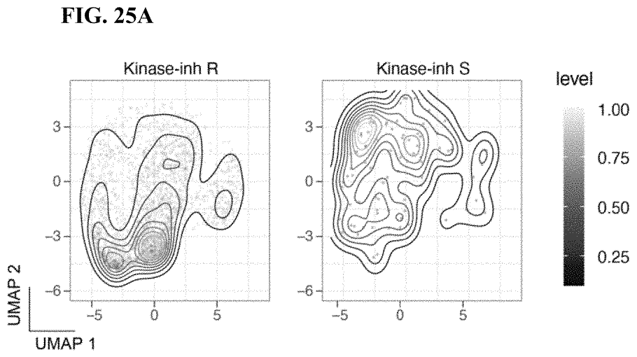

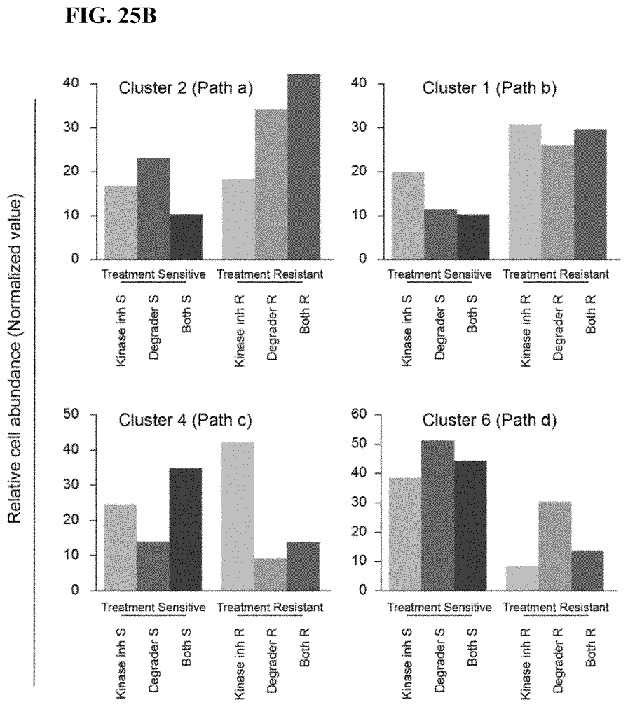

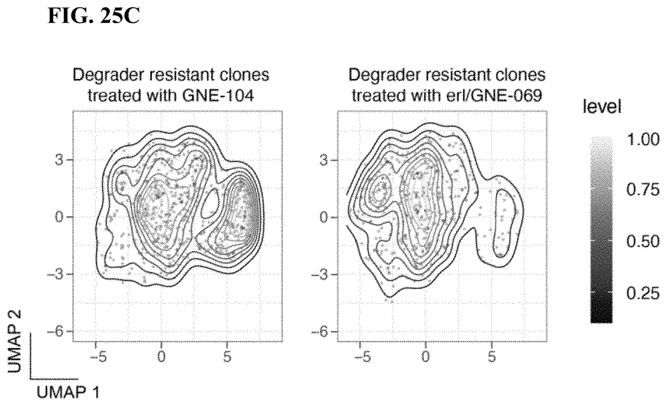

To further explore the adaptive transcriptional programs associated with kinase inhibitor- or degrader-treatment resistance, trajectory inference analysis was performed by systemically ordering treated cells through pseudotime to track cell state evolution in an unsupervised manner. This analysis identified 4 paths (a-d) (FIGS. 18D-18F) with distinct processes and fates. The distribution of kinase inhibitor-sensitive versus kinase inhibitor-resistant clones suggested that paths (b) and (c) are associated with kinase inhibitor resistance, while path (d) represented sensitivity (FIGS. 25A-25B). Consistently, it was found that VIM expression was elevated along paths (b) and (c), representing general resistance to EGFR inhibition (FIG. 18F), while MAPK pathway activity scores were most strongly suppressed and GO/quiescent cell state were most elevated along path (d), consistent with sensitivity. In contrast to the distribution of kinase-inhibitor resistant clones, it was found that degrader-resistant clones were most prevalent along path (a) instead (FIG. 18G). Moreover, while these clones are prominently distributed to at the end of path (a) under to GNE-104 treatment, they had an increased presence in the sensitive trajectory (d) under kinase inhibitor treatment (FIG. 25C). Intriguingly, genes associated with protein processing in ER pathway were uniquely downregulated along path (a) (FIG. 18F), suggesting that reduced expression of ER protein processing genes was uniquely associated with treatment adaptation and survival of these cells under EGFR degrader treatment.

To validate whether reduced expression of ER protein processing genes is a major feature of the adaptive transcriptional responses of degrader resistance clones subject to GNE-104 treatment compared to kinase inhibitors, unbiased gene set expression analysis was conducted. Indeed, it was uncovered that protein processing in ER pathway was the most enriched and most significant pathway induced by erlotinib treatment compared to GNE-104 treatment in these degrader resistant clones (FIG. 18H, Table 2). Taken together, this suggests that cells with pre-existing lower expression of ER protein processing genes are poised to resist EGFR degrader; and a failure to activate these ER protein processing genes by the degrader to the levels observed with the kinase inhibitor may underlie the paradoxical loss of efficacy with our dual inhibitor-degrader EGFR targeted agent.

TABLE 2

| | | | Enrichment | | |
|---|---|---|---|---|---|
| ID | Description | setSize | Score | NES | qvalues |
| hsa04141 | Protein processing in endoplasmic | 140 | 0.47473 | 1.72523 | 0.00730 |
| | reticulum | | | | |
| hsa05167 | Kaposi sarcoma-associated | 131 | 0.47422 | 1.70629 | 0.00730 |
| | herpesvirus infection | | | | |
| hsa05163 | Human cytomegalovirus infection | 153 | 0.44438 | 1.64358 | 0.00928 |
| hsa05169 | Epstein-Barr virus infection | 144 | 0.44086 | 1.60791 | 0.00928 |
| hsa05168 | Herpes simplex virus 1 infection | 319 | 0.35672 | 1.44647 | 0.01114 |
| hsa05165 | Human papillomavirus infection | 228 | 0.37182 | 1.44290 | 0.01114 |
| hsa05132 | Salmonella infection | 172 | 0.37948 | 1.42646 | 0.00928 |
| hsa04810 | Regulation of actin cytoskeleton | 140 | 0.38674 | 1.40545 | 0.02576 |
| hsa04510 | Focal adhesion | 134 | 0.38165 | 1.38228 | 0.03071 |
| hsa05131 | Shigellosis | 173 | 0.36673 | 1.37960 | 0.02576 |
| hsa04218 | Cellular senescence | 131 | 0.37038 | 1.33267 | 0.03945 |
| hsa04144 | Endocytosis | 191 | 0.35072 | 1.33255 | 0.03872 |
| hsa05203 | Viral carcinogenesis | 140 | 0.36510 | 1.32682 | 0.03945 |
| hsa05200 | Pathways in cancer | 323 | 0.32067 | 1.30155 | 0.02663 |
| hsa05170 | Human immunodeficiency virus 1 | 154 | 0.34770 | 1.28844 | 0.04653 |
| | infection | | | | |
| hsa05130 | Pathogenic Escherichia coli | 138 | 0.35236 | 1.28485 | 0.05029 |
| | infection | | | | |
| hsa05166 | Human T-cell leukemia virus 1 | 159 | 0.33354 | 1.24244 | 0.05531 |
| | infection | | | | |
| hsa04140 | Autophagy - animal | 120 | 0.33411 | 1.18894 | 0.10189 |
| hsa05205 | Proteoglycans in cancer | 143 | 0.30636 | 1.11702 | 0.14699 |
| hsa04151 | PI3K-Akt signaling pathway | 192 | 0.27513 | 1.04527 | 0.21371 |
| hsa05010 | Alzheimer disease | 223 | −0.29259 | −1.13529 | 0.10714 |
| hsa05206 | MicroRNAs in cancer | 129 | −0.31502 | −1.14077 | 0.13886 |
| hsa03010 | Ribosome | 129 | −0.35221 | −1.27544 | 0.05531 |
| hsa05016 | Huntington disease | 185 | −0.37296 | −1.41352 | 0.01573 |
| hsa05012 | Parkinson disease | 180 | −0.40388 | −1.52622 | 0.00730 |
| hsa03013 | RNA transport | 139 | −0.51049 | −1.86033 | 0.00730 |
| hsa03040 | Spliceosome | 125 | −0.64427 | −2.33602 | 0.00730 |

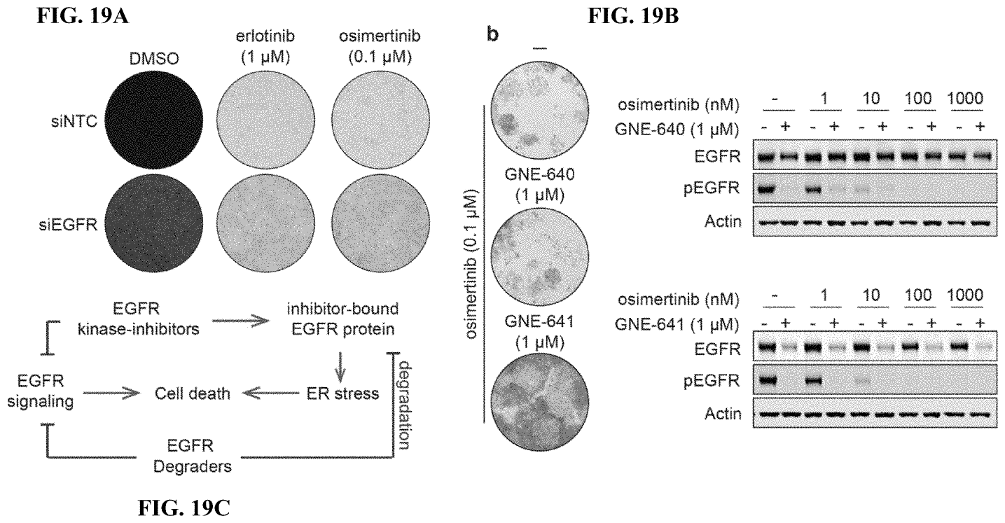

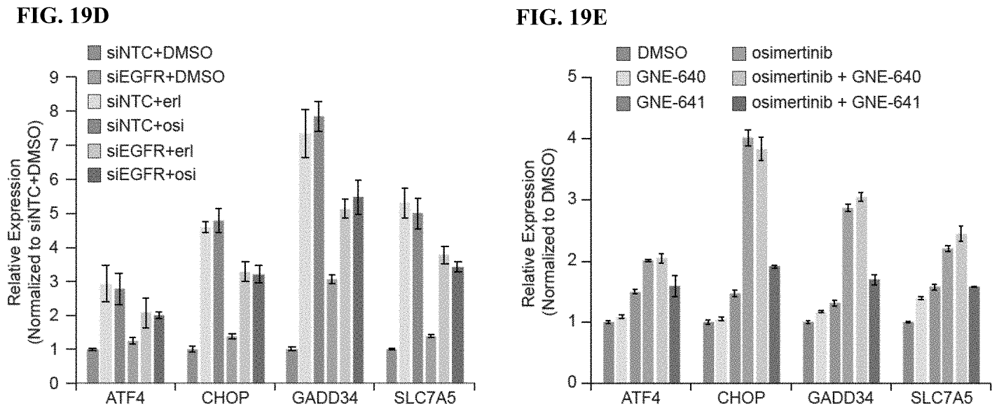

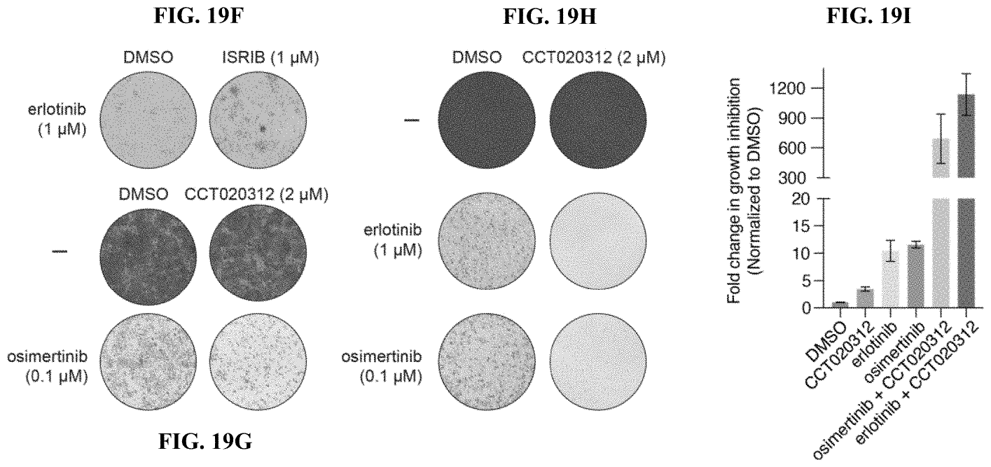

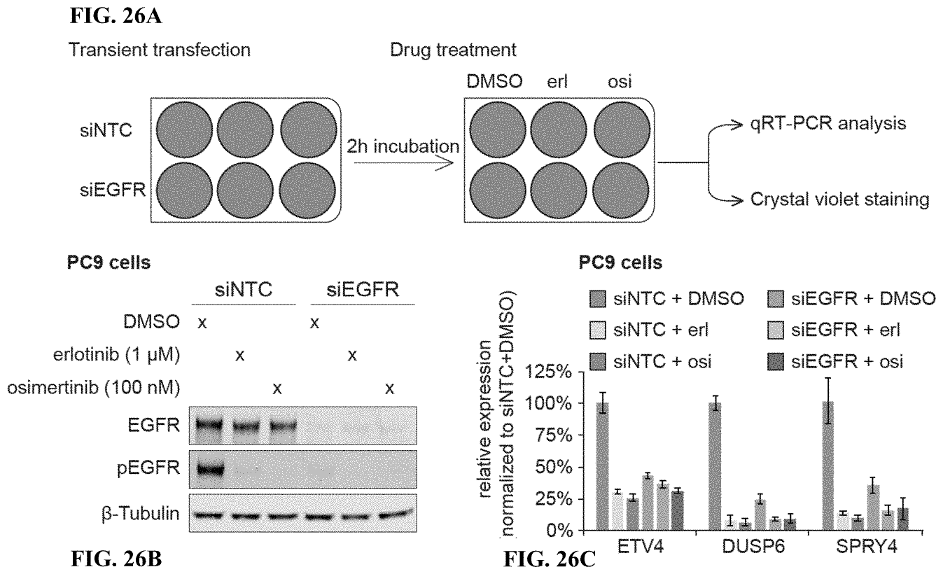

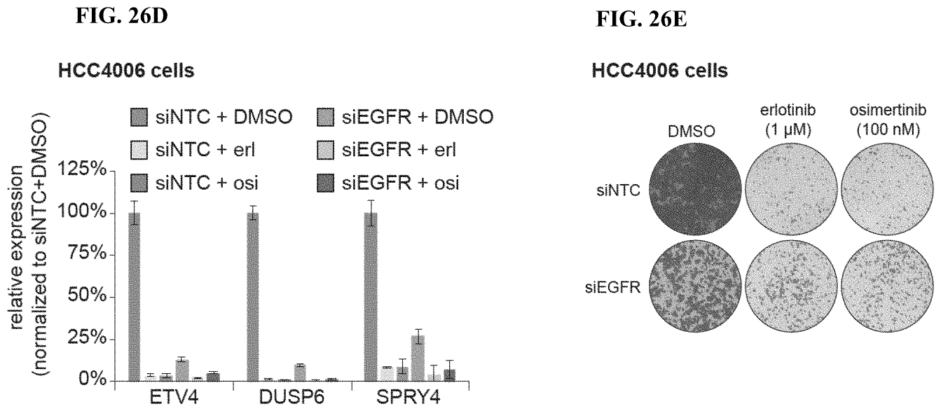

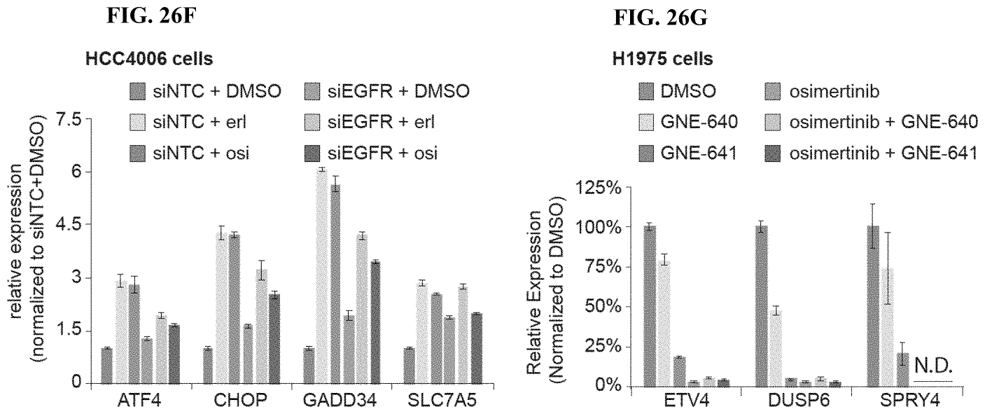

A clear difference between kinase inhibitor versus degrader treatment is the sustained presence of EGFR protein. To test whether the presence of EGFR protein explains these differences, production of EGFR protein was abolished using siRNA in conjunction with kinase inhibitor treatment (FIG. 26A). Although EGFR knockdown alone resulted in substantial loss of EGFR protein (FIG. 26B) as well as strong MAPK pathway suppression (FIGS. 26C, 26D), siRNA treatment was far less effective in killing PC9 and HCC4006 cells (FIGS. 19A, 26E). Moreover, cells treated with siEGFR were paradoxically protected against treatment by kinase inhibitors erlotinib and osimertinib (FIGS. 19A, 26D). These results further suggest that reduction of EGFR protein levels may paradoxically impede cellular efficacy of an EGFR kinase inhibitor.

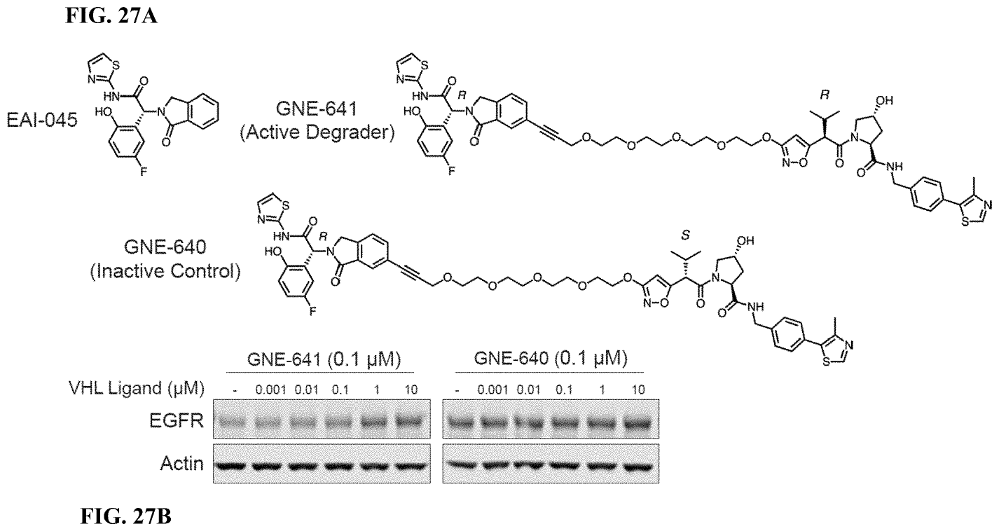

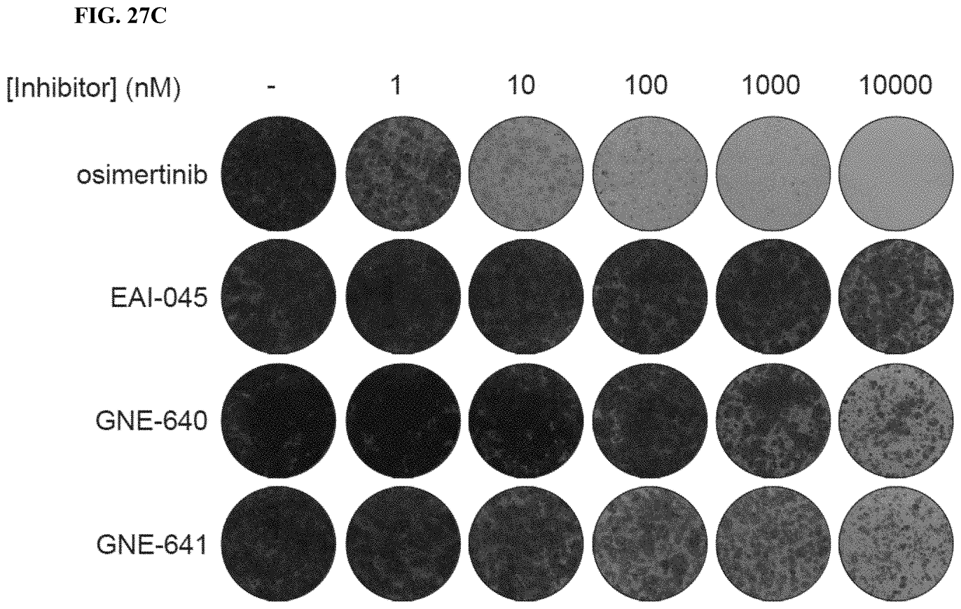

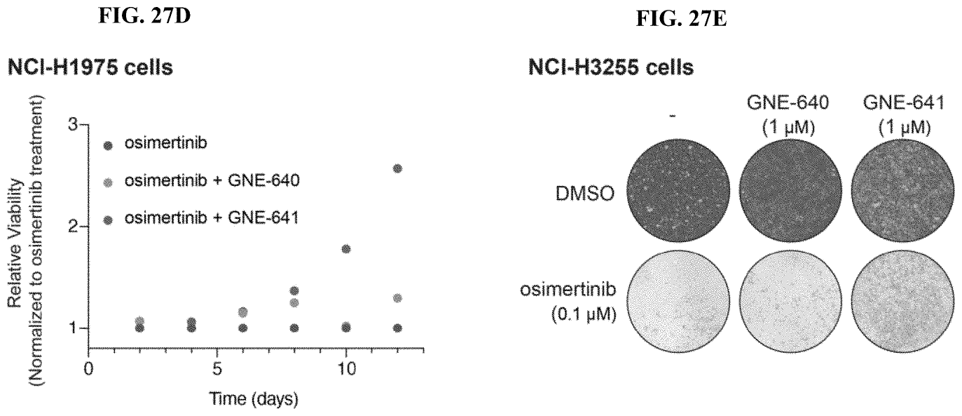

To further test this hypothesis, the EGFR degrader GNE-641 was generated based upon the allosteric EGFR ligand EAI-045 linked to a VHL binding ligand (FIGS. 27A, 27B). EAI-045 was predicted to be capable of binding to EGFR L858R simultaneously with osimertinib based upon molecular modelling (data not shown) and on a recent report. Consistent with modeling predictions, EGFR was efficiently degraded in cells were co-treated with osimertinib & GNE-641 (FIG. 19B). As before, an inactive degrader control was also generated (GNE-640, FIGS. 27A, 27B) which showed no effects on EGFR protein levels (FIG. 19B). Despite its ability to degrade EGFR, GNE-641 exhibited very modest anti-growth activity on its own (FIG. 27C). Yet strikingly, co-treatment of GNE-641 with osimertinib promoted survival of both NCI-1975 and NCI-H3255 cells (relative to osimertinib alone or co-treatment of osimertinib with inactive GNE-640, FIGS. 27D, 27E) despite robust biochemical activity and obvious effect of MAPK pathway target genes (FIGS. 19B, 26G). Taken together, these results strongly indicate that the EGFR protein itself plays a crucial role in mediating cellular efficacy of EGFR kinase inhibitors.

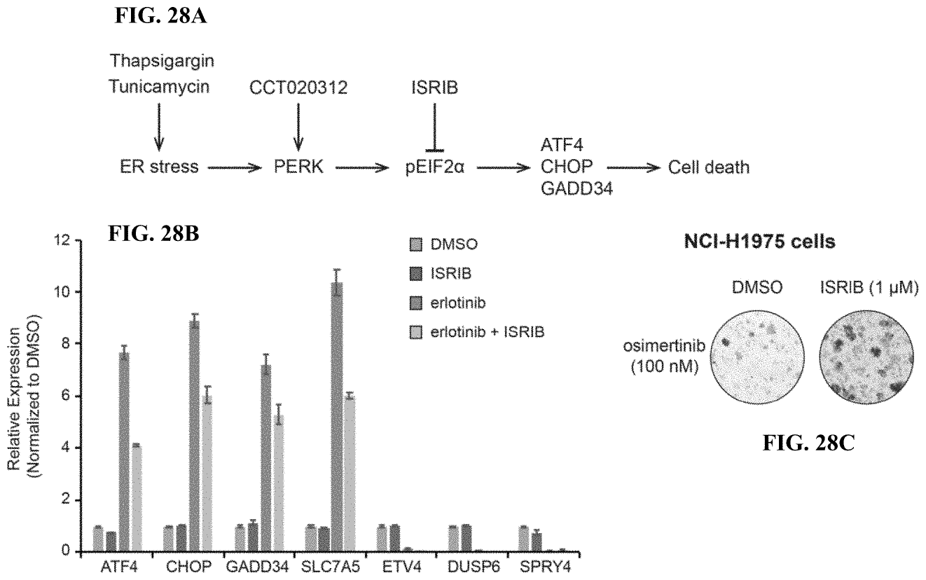

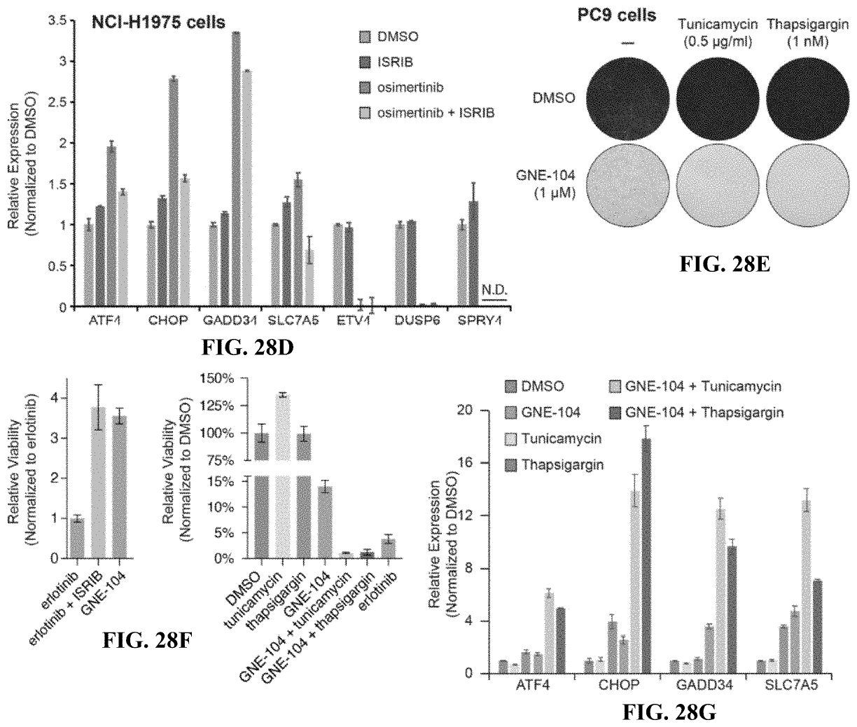

EGFR is a transmembrane protein that traffics through the vesicular system (including ER) through its life cycle. Given that both heightened expression of protein processing genes in ER and sustained presence of EGFR proteins are strongly associated with full efficacy of EGFR kinase inhibitors, we reasoned that pro-death signals initiating from the ER compartment may be responsible for the enhanced efficacy of EGFR inhibitors (relative to degraders). Specifically, disruption of ER proteostasis, such as an increase in protein misfolding and secretion burden, is known to increase ER stress. When ER stress surpasses a certain threshold, it elicits an effective pro-death signal through the ISR PERK-ATF4-CHOP axis (FIG. 28A). We hypothesized that the persistent presence of kinase inhibitor-bound EGFR protein could lead to activation of the PERK-ATF4-CHOP pro-death signaling cascade downstream of ER stress, therefore contributing to cytotoxicity. In contrast, EGFR degraders actively remove inhibitor-bound EGFR protein, thus minimizing the ER stress induced by inhibitor-bound EGFR (FIG. 19C). Indeed, it was found that erlotinib and osimertinib significantly induced expression of key pro-death ISR genes downstream of ER stress (ATF4, CHOP, GADD34) in both PC9, HCC4006 and NCI-H1975 cells, and a major transcriptional target of ATF4 (SLC7A5) PC9 and NCI-H1975 cells (FIGS. 19D, 19E, 26F). Furthermore, such induction was blunted upon acute depletion of EGFR protein production by siEGFR (FIGS. 19D, 26F) or allosteric degrader (FIG. 19E).

To further validate the role of ER stress in EGFR kinase inhibitor efficacy, two questions were asked: 1) Does blocking induction of ATF4 confer resistance to EGFR kinase inhibitors? 2) Does pharmacological induction of ER stress make up for the lost efficacy of an EGFR degrader? To specifically block excessive activation of the pro-death genes downstream of ER stress, cells were sequentially treated with the ISR inhibitor (ISRIB) for 24 hours before adding an EGFR kinase inhibitor. ISRIB treatment indeed blunted activation of ATF4, CHOP, and GADD34 induced by EGFR kinase inhibitors (FIGS. 28A, 28B, 28D), and decreased cell killing in both PC9 and H1975 cells (FIG. 19F, FIGS. 28C, 28F). Conversely, addition of ER stress inducers tunicamycin or thapsigargin (FIG. 28A) at very low non-toxic concentrations greatly induced pro-death ER stress genes and enhanced the cytotoxic activity of the degrader GNE-104 in PC9 cells (FIGS. 28E-28G).

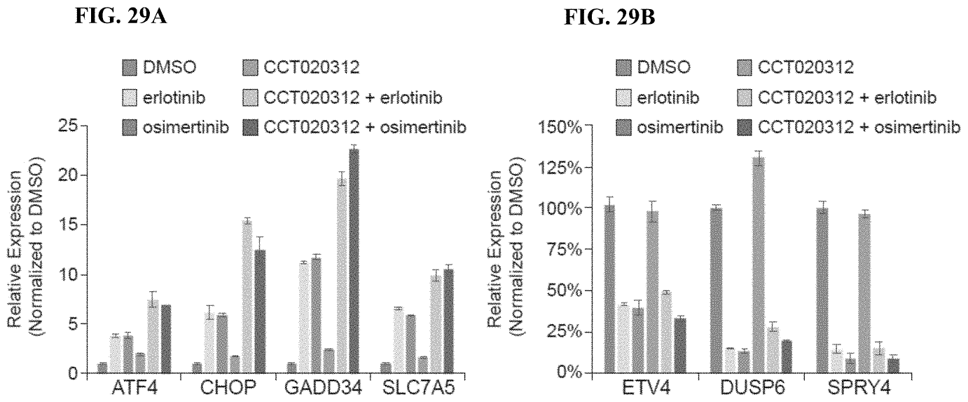

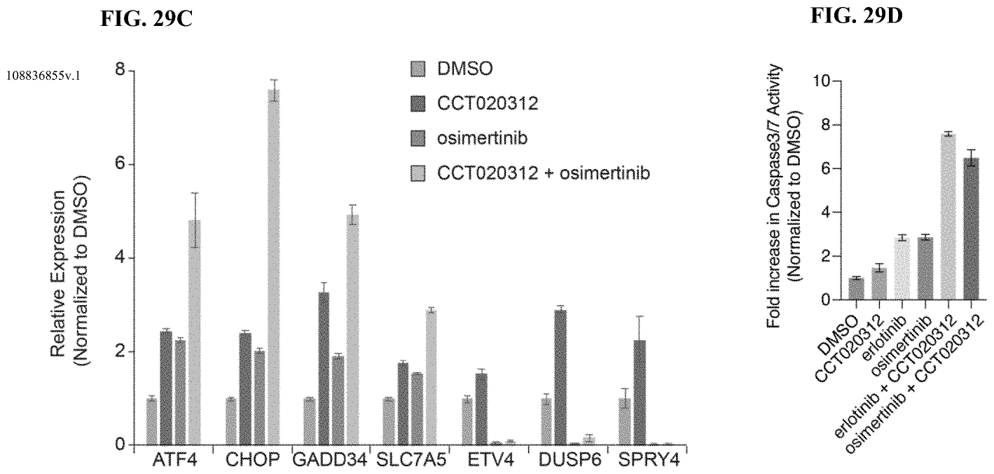

Inspired by these findings, it was explored whether direct activation of the ISR could further enhance the efficacies of clinically approved EGFR inhibitors. The PERK activator CCT020312 was employed here, which specifically activates the PERK-ISR branch downstream of ER stress compared to the more broadly acting ER stress inducers tunicamycin and thapsigargin. When applied in combination with erlotinib or osimertinib, CCT020312 further boosted induction of ISR genes by erlotinib and osimertinib (FIGS. 29A-29C) and enhanced cell death induction (FIG. 29D) resulting in effectively eliminated residual cells that commonly remain after acute EGFR inhibitor treatment (FIGS. 19G-19I). These results highlighted a previously unappreciated avenue for boosting response to anti-EGFR therapies that could be of clinical interest.

In summary, the TraCe-seq platform enables identification of pre-existing and adaptive transcriptional features that impact the outcome of different therapeutic treatments. By tracing the cells that responded and persisted after exposure to different EGFR targeted therapies and conducting differential gene analyses coupled with pseudotime trajectory comparisons, unexpected differences were uncovered between the cellular mechanisms of EGFR targeted agents with differential MOAs. This revealed an exciting and previously unappreciated component underlying EGFR inhibitor response: the induction of ER stress due to inhibitor-bound EGFR is essential for achieving full efficacy. Detailed understanding of the biochemical mechanisms underlying inhibitor-bound EGFR and ER stress induction could aid future development of small molecules against EGFR and perhaps other membrane associated proteins. Further, the results challenge a wide-spread assumption that target degradation will be universally superior to occupancy-based inhibition, suggesting underlying biology needs to be carefully considered when selecting compound MOA.

The TraCe-seq approach will guide development of future therapies by revealing unknown features that predict response and resistance to different molecular modalities or treatment combinations. In addition, as the barcodes are stably integrated and constitutively expressed, the TraCe-seq barcoded population could be sampled at multiple time points to gather transcriptional information with clonal resolution over time, thus providing a generally applicable approach for studying evolution of heterogeneous population under a variety of contexts. In addition, additional modification of the method to allow direct isolation of live cells within pre-existing subpopulations of interest could further broaden its usage across diverse systems.

### Methods

Cell lines and tissue culture: Cell lines were obtained, characterized, and quality controlled as described. Cell lines were maintained using standard tissue culture technics. All cell lines (except for MCF-10A) were cultured in RPMI with 10% heat inactivated fetal bovine serum and 2 mM L-Glutamine. MCF10-A cells were cultured in DMEM-F12 (Thermo Fisher Scientific, 11330-032), 5% Horse serum (Sigma Aldrich, H1270), 10 μg/ml insulin (Sigma Aldrich, I-1882), 500 ng/ml hydrocortisone (Sigma Aldrich, H-0888), 100 ng/ml cholera toxin (Sigma Aldrich, C-8052), and 20 ng/ml EGF (Peprotech, 100-15R).

Lentivirus production and cell transduction: TraCe-Seq lentivirus barcode library was synthesized by Cellecta Inc. Lentivirus was produced in 293T cells by co-transfecting the barcode library with pCMVdR8.9 (expressing gag, pol, and rev genes) and pCMV-VSV-g (expressing envelope protein). Virus were concentrated using Lenti-X-Concentrator and stored at −80° C. For virus transduction, 5×10⁶ cells were seeded in T75 flask and infected overnight with 8 μg/ml polybrene (TR-1003-G, EMD Millipore) at MOI (multiplexity of infection) at 0.05-0.1. Cells were first split at two days after virus infection. To determine the MOI, a subfraction of cells were plated to a 96-well plate at 5,000 cells/well in 100 μl media. Cells were then incubated with or without 2 μg/ml puromycin, and the proportion of infected, puromycin-resistant cells was determined by a viability assay using the CellTiter-Glo Luminescent Cell Viability Assay (Promega Cat. No. G7572) to confirm the infection MOI was within range. The rest of the cells were expanded in 2 μg/ml puromycin.

Enrichment of eGFP-expressing cells by FACS TraCe-seq library transduced, puromycin-selected cells were dissociated into single cells and resuspended at 10×10⁶/ml in PBS. Top 50% eGFP expressing cells were collected on a BD Aria Fusion cell sorter expanded in cell culture media with 2 μg/ml puromycin.

Single cell RNA sequencing: Cultured cells were trypsinized into single cell suspensions and processed using Chromium Single Cell Gene Expression 3′ Library and Gel Bead Kit following the manufacturer's instructions (10×Genomics, Pleasanton, Calif.). Cells were counted and checked for viability using Vi-CELL XR cell counter (Beckman Coulter, Brea, Calif.), and then injected into microfluidic chips to form Gel Beads-in-Emulsion (GEMs) in the 10×Chromium instrument. Reverse transcription was performed on the GEMs, and RT products were purified and amplified. Expression libraries were made from the cDNA and profiled using the Bioanalyzer High Sensitivity DNA kit (Agilent Technologies, Santa Clara, Calif.) and quantified with Kapa Library Quantification Kit (Kapa Biosystems, Wilmington, Mass.). Illumina HiSeq2500 or HiSeq4000 (Illumina, San Diego, Calif.) was used to sequence the libraries.

### Chemical Synthesis

VHL Ligands: VHL ligand (GNE-429) used in the competition assay in FIG. 21B was synthesized as previously described. Active and inactive VHL ligands (GNE-128 and GNE-127, respectively) used in FIG. 17C and FIG. 27A were synthesized as previously described. Note, the stereochemistry of these two compounds was assigned based on biochemical potency (vs literature) with GNE-128 as the active isomer.

Erlotinib-Derived Degraders:

### 4-((3-Ethynylphenyl)amino)-7-(2-methoxyethoxy) quinazolin-6-ol

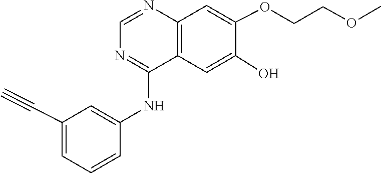 [SMILES: C#Cc1cccc(Nc2ncnc3cc(OCCOC)c(O)cc23)c1]

4-((3-Ethynylphenyl)amino)-7-(2-methoxyethoxy) quinazolin-6-ol was prepared similar to as described previously (4). As a final step, a solution of 7-(2-methoxyethoxy)-4-((3-((trimethylsilyl) ethynyl)phenyl)amino)quinazolin-6-ol (1.23 g, 3.02 mmol) and TBAF (6 mL, 1 M in THF) in THF (10 mL) was stirred at room temperature for 20 mins. Concentrated under vacuum. The residue was purified by flash chromatography on silica gel eluting with ethyl acetate/petroleum ether (0-100%) to afford 560 mg (55% yield) of the title compound as a light yellow solid. LCMS (ESI): [M+H]⁺=336. ¹H NMR (300 MHz, DMSO-d6) δ 9.65 (s, 1H), 9.43 (s, 1H), 8.47 (s, 1H), 8.08 (t, J=1.9 Hz, 1H), 7.92-7.85 (m, 1H), 7.80 (s, 1H), 7.37 (t, J=7.9 Hz, 1H), 7.20 (s, 1H), 7.18-7.12 (m, 1H), 4.36-4.26 (m, 2H), 4.19 (s, 1H), 3.83-3.73 (m, 2H), 3.35 (s, 3H).

### (2S,4S)-1-((S)-2-Amino-3,3-dimethylbutanoyl)-4-hydroxy-N-(4-(4-methylthiazol-5-yl)benzyl)pyrrolidine-2-carboxamide hydrogen chloride

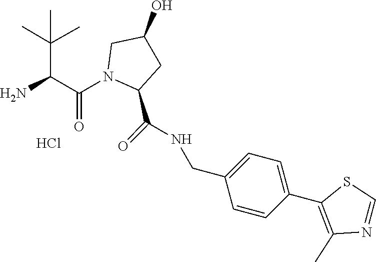 [SMILES: Cc1ncsc1-c1ccc(CNC(=O)[C@@H]2C[C@H](O)CN2C(=O)[C@@H](N)C(C)(C)C)cc1 ; Cl]

(2S,4S)-1-((S)-2-Amino-3,3-dimethylbutanoyl)-4-hydroxy-N-(4-(4-methylthiazol-5-yl)benzyl)pyrrolidine-2-carboxamide hydrogen chloride was prepared similar to as described for (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide using 1-tert-butyl 2-methyl (2S,4S)-4-hydroxypyrrolidine-1,2-dicarboxylate as starting material (2). In a final step, a solution of tert-butyl N-[(2S)-1-[(2S,4S)-4-hydroxy-2-([[4-(4-methyl-1,3-thiazol yl)phenyl]methyl]carbamoyl)pyrrolidin-1-yl]-3,3-dimethyl-1-oxobutan-2-yl]carbamate (718 mg, 1.35 mmol) in dichloromethane (6 mL) and HCl/1,4-dioxane (3 mL, 4M) was stirred for 0.5 h at 25° C. The resulting mixture was concentrated under vacuum. The residue was stirred with ethyl ether (5 mL). The solid was collected by filtration and dried under vacuum to afford 700 mg (crude) of the title compound as a white solid. LCMS (ESI): [M+H]+=431.3. 1H NMR (300 MHz, DMSO-d6) δ 9.04 (s, 1H), 8.79 (t, J=6.0 Hz, 1H), 8.16 (s, 3H), 7.41 (s, 4H), 4.52-4.18 (m, 5H), 4.02 (dd, J=10.3, 5.9 Hz, 1H), 3.94 (d, J=5.5 Hz, 1H), 3.31 (dd, J=10.2, 6.6 Hz, 1H), 2.45 (s, 3H), 2.42-2.30 (m, 1H), 1.74 (dt, J=12.5, 7.3 Hz, 1H), 1.03 (s, 9H).

### Synthetic Scheme for Preparation of Ethyl 2-((6-(2-bromoethoxy)hexa-2,4-diyn-1-yl)oxy)acetate

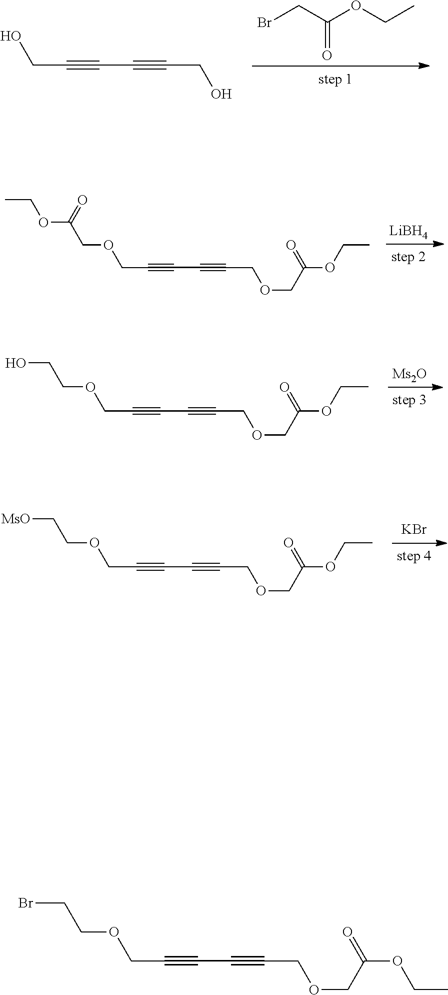

### Diethyl 2,2′-(hexa-2,4-diyne-1,6-diylbis(oxy))diacetate

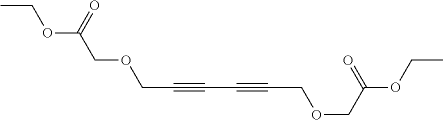 [SMILES: CCOC(=O)COCC#CC#CCOCC(=O)OCC]

To a solution of hexa-2,4-diyne-1,6-diol (1 g, 9.08 mmol) in tetrahydrofuran (20 mL) was added sodium hydride (1.08 g, 45.0 mmol) under nitrogen. Stirred for 1 h at 25° C. under nitrogen atmosphere. Then ethyl 2-bromoacetate (4.50 g, 26.9 mmol) was added and the resulting solution was stirred for 3 h at 25° C. Concentrated under vacuum. The residue was purified by flash chromatography on silica gel column eluting with ethyl acetate/petroleum ether (0%-50%) to afford 1.3 g (51%) of title compound as colorless oil. LCMS (ESI): [M+18]+=300.

### Ethyl 2-((6-(2-hydroxyethoxy)hexa-2,4-diyn-1-yl)oxy)acetate

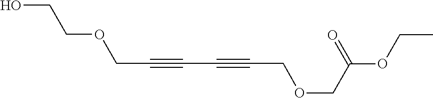 [SMILES: CCOC(=O)COCC#CC#CCOCCO]

To a solution of ethyl 2-[[6-(2-ethoxy-2-oxoethoxy)hexa-2,4-diyn-1-yl]oxy]acetate (1.10 g, 3.90 mmol) in tetrahydrofuran (10 mL) was added LiBH₄ (86.0 mg, 3.95 mmol) at 0° C. The resulting solution was stirred for 3 h at 25° C. The reaction was quenched by the addition of ethyl acetate. The resulting mixture was concentrated under vacuum. The residue was partitioned between ethyl acetate and water. The organic layers were dried over anhydrous sodium sulfate and concentrated under vacuum. The residue was purified by flash chromatography on silica gel column eluting with ethyl acetate/petroleum ether (0%-80%) to afford 237 mg (25%) of the title compound as colorless oil. LCMS (ESI): [M+H]⁺=241.

### Ethyl 2-((6-(2-((methylsulfonyl)oxy)ethoxy)hexa-2,4-diyn-1-yl)oxy)acetate

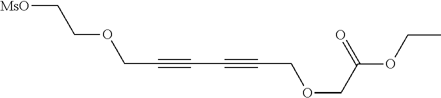 [SMILES: CCOC(=O)COCC#CC#CCOCCOS(C)(=O)=O]

A solution of ethyl 2-[[6-(2-hydroxyethoxy)hexa-2,4-diyn-1-yl]oxy]acetate (237 mg, 0.986 mmol), methanesulfonyl methanesulfonate (348 mg, 2.00 mmol) and DIPEA (516 mg, 3.99 mmol) in dichloromethane (4 mL) was stirred for 1 h at 25° C. The resulting mixture was washed with water, dried and concentrated. This resulted in 320 mg (crude) of title compound as brown oil. LCMS (ESI): [M+H]⁺=319. The crude was used for next step without further purification.

### Ethyl 2-((6-(2-bromoethoxy)hexa-2,4-diyn-1-yl)oxy)acetate

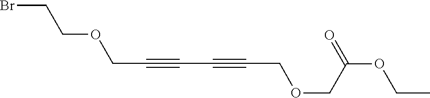 [SMILES: CCOC(=O)COCC#CC#CCOCCBr]

A solution of ethyl 2-([6-[2-(methanesulfonyloxy)ethoxy]hexa-2,4-diyn-1-yl]oxy)acetate (320 mg, 1.005 mmol), KBr (354 mg, 2.98 mmol) in N,N-dimethylformamide (3 mL) was stirred overnight at 60° C. Cooled to room temperature. The reaction mixture was partitioned between ethyl acetate and water. The organic layers were dried over anhydrous sodium sulfate and concentrated under vacuum. The residue was purified by flash chromatography on silica gel column with ethyl acetate/petroleum ether (0%-40%) to afford 222 mg (73%) the title compound as colorless oil. LCMS (ESI): [M+H]⁺=303/305. 1H NMR (300 MHz, DMSO-d6) δ 4.43-4.34 (m, 4H), 4.19-4.06 (m, 4H), 3.82-3.73 (m, 2H), 3.66-3.56 (m, 2H), 1.20 (t, J=7.1 Hz, 3H).

### Synthetic Scheme for the Preparation of GNE-104, (2R,4R)-1-((S)-2-(2-((6-(2-((4-((3-Ethynylphenyl)amino)-7-(2-methoxyethoxy)quinazolin-6-yl)oxy)ethoxy)hexa-2,4-diyn-1-yl)oxy)acetamido)-3,3-dimethylbutanoyl)-4-hydroxy-N-(4-(4-methylthiazol-5-yl)benzyl)pyrrolidine-2-carboxamide

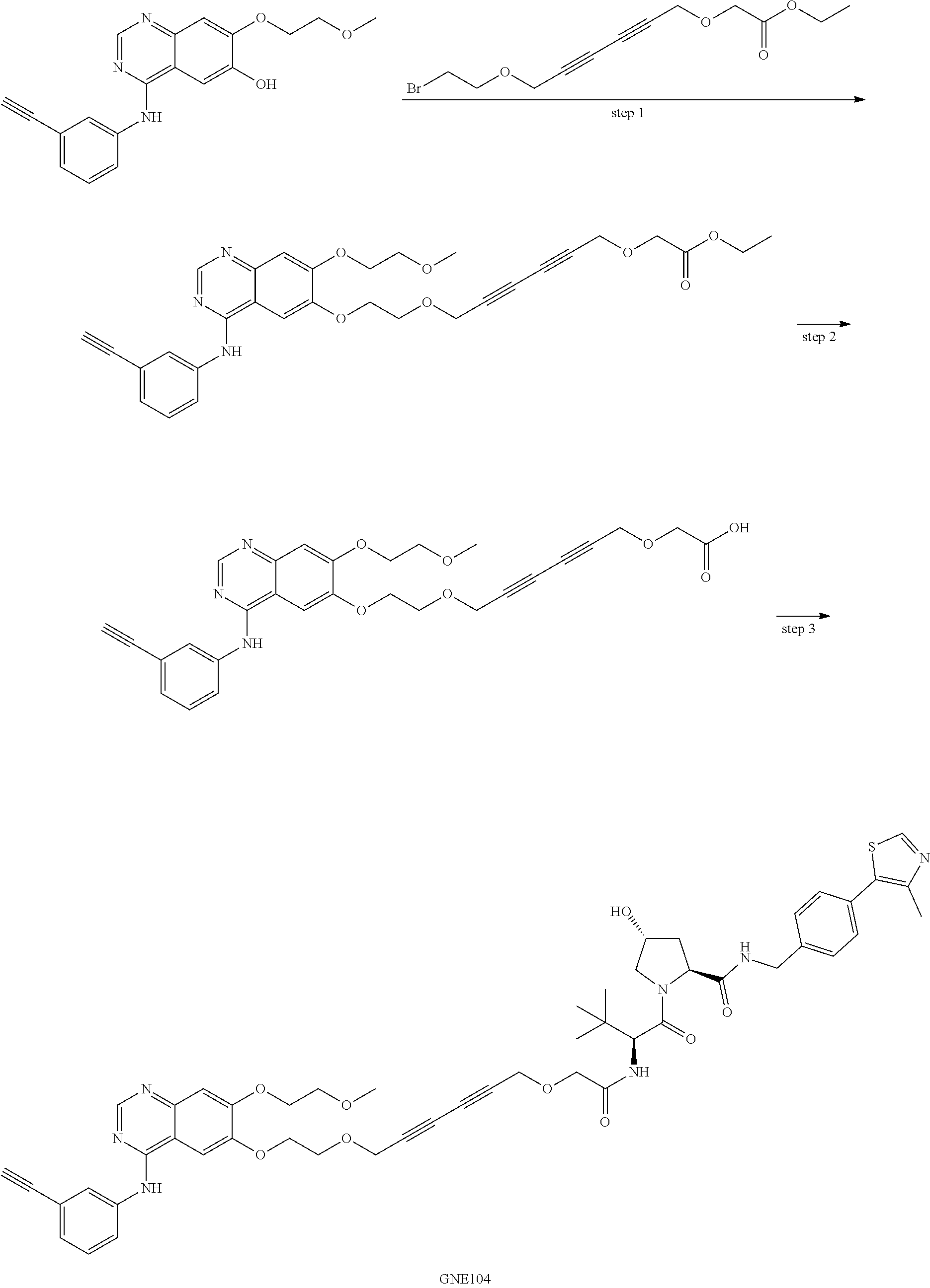

### Ethyl 2-((6-(2-((4-((3-ethynylphenyl)amino)-7-(2-methoxyethoxy)quinazolin-6-yl)oxy)ethoxy)hexa-2,4-diyn-1-yl)oxy)acetate

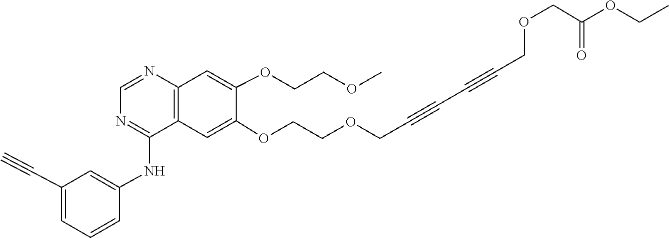 [SMILES: C#Cc1cccc(Nc2ncnc3cc(OCCOC)c(OCCOCC#CC#CCOCC(=O)OCC)cc23)c1]

A solution of 4-(3-ethynylanilino)-7-(2-methoxyethoxy)quinazolin-6-ol (50.0 mg, 0.150 mmol), ethyl 2-[6-(2-bromoethoxy)hexa-2,4-diynoxy]acetate (67.8 mg, 0.220 mmol) and K₂CO₃ (41.2 mg, 0.300 mmol) in N,N-dimethylformamide (2 mL) was stirred at 60° C. for 2 hours. The reaction solution was loaded onto reverse phase column directly eluting with CH₃CN/H₂O (0.5% NH₄HCO₃) (0-100%) to afford title compound (46 mg, 55.3% yield) as a white solid. LCMS (ESI): [M+H]⁺=558

### 2-((6-(2-((4-((3-Ethynylphenyl)amino)-7-(2-methoxyethoxy)quinazolin-6-yl)oxy)ethoxy)hexa-2,4-diyn-1-yl)oxy)acetic acid

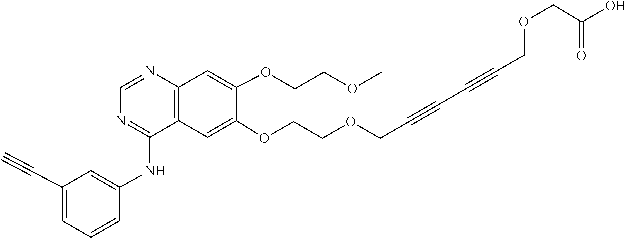 [SMILES: C#Cc1cccc(Nc2ncnc3cc(OCCOC)c(OCCOCC#CC#CCOCC(=O)O)cc23)c1]

A solution of ethyl 2-[6-[2-[4-(3-ethynylanilino)-7-(2-methoxyethoxy)quinazolin-6-yl]oxyethoxy]hexa-2,4-diynoxy]acetate (46.0 mg, 0.0800 mmol) and LiOH (5.94 mg, 0.250 mmol) in tetrahydrofuran (2 mL) and water (1 mL) was stirred at 25° C. for 1 hours. Concentrated under vacuum. The residue was purified by reverse phase column eluting with CH₃CN/H₂O (0.5% NH₄HCO₃) (0-100%) to afford the title compound (40 mg, 91.6% yield) as a white solid. LCMS (ESI): [M+H]+=530

### GNE-104, (2R,4R)-1-((S)-2-(2-((6-(2-((4-((3-Ethynylphenyl)amino)-7-(2-methoxyethoxy)quinazolin-6-yl)oxy)ethoxy)hexa-2,4-diyn-1-yl)oxy)acetamido)-3,3-dimethylbutanoyl)-4-hydroxy-N-(4-(4-methylthiazol-5-yl)benzyl)pyrrolidine-2-carboxamide

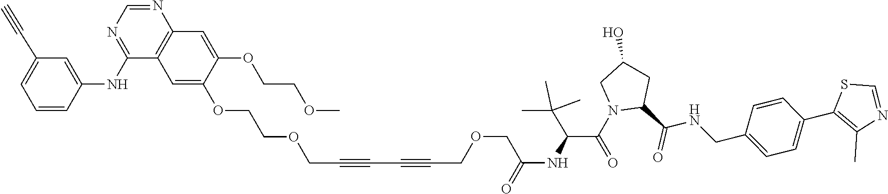

To a solution of 2-[6-[2-[4-(3-ethynylanilino)-7-(2-methoxyethoxy)quinazolin-6-yl]oxyethoxy]hexa-2,4-diynoxy]acetic acid (40.0 mg, 0.080 mmol), (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (41.1 mg, 0.080 mmol) and DIPEA (10.3 mg, 0.080 mmol) in N,N-Dimethylformamide (2 mL) was added HATU (28.7 mg, 0.080 mmol). The resulting solution was stirred at 25° C. for 1 hours. The solution was loaded onto reverse phase column directly eluting with CH₃CN/H₂O (0.1% FA) (5-100%) to afford the title compound (29 mg, 40.8% yield) as a white solid. LCMS (ESI): [M+H]⁺=942. ¹H NMR (400 MHz, DMSO-d₆) δ 9.47 (s, 1H), 8.98 (s, 1H), 8.60 (t, J=6.1 Hz, 1H), 8.50 (s, 1H), 7.99 (t, J=1.9 Hz, 1H), 7.93-7.84 (m, 2H), 7.56 (d, J=9.4 Hz, 1H), 7.48-7.36 (m, 5H), 7.26-7.17 (m, 2H), 5.15 (d, J=3.6 Hz, 1H), 4.56-4.18 (m, 14H), 4.03 (s, 2H), 3.95-3.88 (m, 2H), 3.78-3.71 (m, 2H), 3.70-3.56 (m, 2H), 3.36 (s, 3H), 2.44 (s, 3H), 2.06 (d, J=8.9 Hz, 1H), 1.96-1.84 (m, 1H), 0.94 (s, 9H).

### GNE-069, (2R,4S)-1-((S)-2-(2-((6-(2-((4-((3-Ethynylphenyl)amino)-7-(2-methoxyethoxy)quinazolin-6-yl)oxy)ethoxy)hexa-2,4-diyn-1-yl)oxy)acetamido)-3,3-dimethylbutanoyl)-4-hydroxy-N-(4-(4-methylthiazol-5-yl)benzyl)pyrrolidine-2-carboxamide

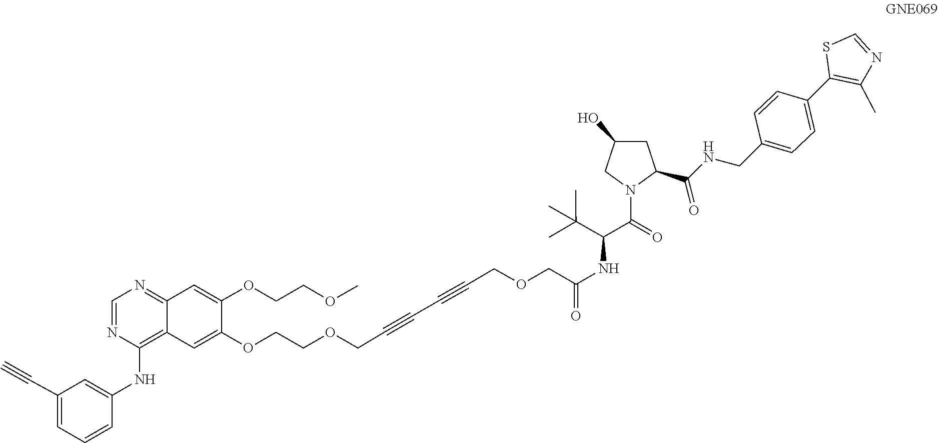 [SMILES: C#Cc1cccc(Nc2ncnc3cc(OCCOC)c(OCCOCC#CC#CCOCC(=O)N[C@H](C(=O)N4C[C@@H](O)C[C@H]4C(=O)NCc4ccc(-c5scnc5C)cc4)C(C)(C)C)cc23)c1]

To a solution of 2-[6-[2-[4-(3-ethynylanilino)-7-(2-methoxyethoxy)quinazolin-6-yl]oxyethoxy]hexa-2,4-diynoxy]acetic acid (60.0 mg, 0.110 mmol), (4S)-1-[(2S)-2-amino-3,3-dimethyl-butanoyl]-4-hydroxy-N-[[4-(4-methylthiazol-5-yl)phenyl]methyl]pyrrolidine-2-carboxamide hydrochloride (52.9 mg, 0.110 mmol) and DIPEA (43.9 mg, 0.340 mmol) in N,N-dimethylformamide (2 mL) was added HATU (47.4 mg, 0.120 mmol). The resulting solution was stirred at room temperature for 1 h. The reaction solution was loaded onto reverse phase column eluting with CH₃CN/H₂O (0.5% NH₄HCO₃) (0-100%) to afford the title compound (59.6 mg 55.8% yield) as a light yellow solid. LCMS (ESI): [M+H]+=942. ¹H NMR (400 MHz, DMSO-d₆) δ 9.47 (s, 1H), 8.98 (s, 1H), 8.66 (t, J=6.0 Hz, 1H), 8.50 (s, 1H), 7.99 (t, J=1.9 Hz, 1H), 7.93-7.85 (m, 2H), 7.58 (d, J=9.0 Hz, 1H), 7.40 (d, J=3.8 Hz, 5H), 7.26-7.17 (m, 2H), 5.44 (d, J=7.2 Hz, 1H), 4.54-4.17 (m, 14H), 4.08-3.95 (m, 2H), 3.90 (dt, J=16.9, 5.2 Hz, 3H), 3.75 (t, J=4.4 Hz, 2H), 3.45 (dd, J=10.1, 5.4 Hz, 1H), 3.35 (s, 3H), 2.44 (s, 3H), 2.35 (m, 1H), 1.74 (dt, J=12.5, 6.1 Hz, 1H), 0.95 (s, 9H).

Allosteric-derived degraders: Synthetic procedures for GNE-640 and GNE-641 are detailed in patent application WO2019183523 (identified as 1002.3 and 1002.4, respectively). Briefly, the desired stereochemistry of the EGFR allosteric ligand was established from the crystal structure of the related ligand EAI-001 bound to EGFR(T790M/V948R). The assigned (R) stereochemistry for GNE-640 and GNE-641 (FIG. 27A) was inferred from relative affinities.

Compound dose matching: GNE-069 and GNE-104 were applied at an equimolar concentration of 1 μM as the two compounds are matched in terms of their anti-EGFR specificity and potency as well as their physicochemical properties. The dose for erlotinib was empirically determined by matching the killing efficiency and kinetics to that induced by 1 μM GNE-069 (FIG. 22C).

Clonogenic assay: Cells were seeded at desired densities in two to three replicates in 6-well plates and allowed to attach overnight. The following day cells were treated with the indicated compounds. Media containing the appropriate compounds was replenished every 48-72 hours. At the end of the assay, the cells were washed once with PBS, fixed and stained with crystal violet solution (Sigma Aldrich, HT90132) for 20 minutes, washed with water, and allowed to dry before scanning. For experiments shown in FIG. 18E erlotinib concentration were determined to match the cell viability effect to 1 μM GNE-069 in each cell line, so that 1 μM erlotinib was applied to PC-9 and HCC2935, and 100 nM erlotinib was applied to HCC4006 and HCC827. For experiments shown in FIG. 27B, cells were treated with either DMSO or 1 μM ISRIB (Tocris Cat. No. 5284) for 24 h, and then co-treated with EGFR inhibitors for 8 days. For other experiments cells were treated with 1 μM GNE-104 for 24 h, and then co-treated with 0.5 μg/ml Tunicamycin (Sigma Aldrich, T7765), or 1 nM Thapsigargin (Sigma Aldrich, T9033) for three days.

Cell viability and cell death quantification: For experiments shown in FIGS. 24C, 27D, the indicated cells were seeded at desired densities in replicates in 24-well plates and allowed to attach overnight. The following day cells were treated with the indicated compounds. Media containing the appropriate compounds was replenished every 48 hours. At each media change, cell viabilities were quantified using CellTiter-Glo Luminescent Cell Viability Assay (Promega, G7572). For experiments shown in FIG. 191 and FIG. 29D, PC9 cells were seeded in replicates in 96-well plates and allowed to attach overnight. The following day cells were treated with the indicated compounds or compound combinations at the indicated concentrations. Induction of cell death was quantified 24 h post-treatment using Caspase-Glo 3/7 Assay System (Promega, G8093) and cell survival were measured 72 h post-treatment using CellTiter-Glo Luminescent Cell Viability Assay (Promega, G7572). CCT020312 was obtained from EMD Millipore (324879).

Immunoblotting: Immunoblotting performed using standard methods. Cells were briefly washed in ice-cold PBS and lysed in the following lysis buffer (1% NP40, 50 mM Tris, pH 7.8, 150 mM NaCl, 5 mM EDTA) plus protease inhibitor mixture (Complete mini tablets, Roche Applied Science, 11836170001) and phosphatase inhibitor mix (Thermo Fisher Scientific, 78420). Lysates centrifuged at 15,000 rpm for 10 minutes at 4° C. and the protein concentration determined by BCA (Thermo Fisher Scientific, 23227). Equal amounts of protein were resolved by SDS-PAGE on NuPAGE, 4-12% Bis-Tris Gels (Thermo Fisher Scientific, WG-1403) and transferred to nitrocellulose membrane (Bio-Rad, 170-4159). Membranes were blocked in blocking buffer (Li-Cor, 927-40000), incubated overnight with the indicated primary antibodies and analyzed by the addition of secondary antibodies IRDye 680LT Goat anti-Mouse IgG (Li-Cor, 926-68050) or IRDye 800CW Goat anti-Rabbit IgG (Li-Cor, 926-32211). The membranes were visualized on a Li-Cor Odyssey CLx Scanner. Anti-EGFR (MI-12-1) purchased from Medical and Biological Labs, anti-pEGFR (3777), anti β-Tubulin (2146) and anti-β-Actin (4970) purchased from Cell Signaling Technology. IR-conjugated secondary antibodies Goat anti-Mouse 680LT (926-68020), and Goat anti-Rabbit 800CW (926-32211) purchased from Li-Cor.

RNA extraction and qRT-PCR: RNA was extracted using Qiagen RNeasy Plus kit following manufacturer's instruction. RNA was reverse transcribed using High-Capacity cDNA Reverse Transcription Kit (Thermo Fisher Scientific Cat. No. 4368814). qPCRs were performed using TaqMan™ Fast Advanced Master Mix (Thermo Fisher Scientific Cat. No. 4444557) on ABI QuantStudio 7 Flex Real-Time PCR Systems. HPRT1 was used as the internal control to quantify relative gene expression levels. Taqman probes were obtained from Thermo Fisher Scientific (Cat. No. 431182, 4448490). These probes were used in the study: ATF4 Hs00909569_g1, DDIT3/CHOP Hs00358796_g1, PPP1R15A/GADD34 Hs00169585_m1, SLC7A5 Hs01001183_m1, ETV4 Hs00385910_m1, SPRY4 Hs00540086_m1, DUSP6 Hs04329643_s1, HPRT1 Hs02800695_m1.

siRNA transfection: PC9 cells were seeded at 200K/well into 6-well plate and allowed to attach overnight. The following day the cells were transfected with siNTC pool (Dharmacon, Inc. Cat. No. D-001810-10-50) or siEGFR (Dharmacon, Inc. equal mix of Cat. No. J-003114-12-0050 and J-003114-13-0050) at a final concentration of 50 nM with Lipofectamin RNAiMAX transfection reagent (Thermo Fisher Scientific, Cat. No. 13778030). DMSO, erlotinib (1 μM), or osimertinib (100 nM) were added two hours after transfection. Media was replenished every 48-72 h afterwards.

scRNA-Seq data processing and analysis: scRNA-Seq data were processed with CellRanger 2.1.0 using mkfastq and count commands. Expression data were processed on the pre-built human reference GRCh38. Cell Ranger performed default filtering for quality control and the data from all samples were combined using Seurat package v.3.0.0 to form an aggregate Seurat object using the Seurat best-practices workflow. Cells were combined into a single Seurat object followed by ScaleData using UMI counts and G2M cell cycle score (Seurat best practices). FindNeighbors, followed by FindClusters and RunUMAP, was performed on the top 10 PCA components using 2,304 genes identified by Find VariableFeatures. Cell clustering was performed using Louvarin with resolution set to 0.5. Differential expression was performed using FindMarkers and Wilcoxon Rank Sum test. Module scores of a given gene set or pathway were calculated on a per-cell basis using Seurat AddModuleScore function.

Long-term resistance assay: An experimental population of ˜500 cells with unique TraCe-seq barcodes was used for the initiating population. This population was expanded for 12 doublings, and then seeded 15,000 cells from this expanded population (representing a 30×coverage of the barcodes) per replicates into long-term resistance studies in replicates. Erlotinib was applied at a highly stringent dose reported in the literature and applied GNE-104 at half of the concentration of erlotinib to represent a less stringent treatment condition. Media was replenished every 48 h. After two months of culture, cell pellets were sent to Cellecta Inc for genomic DNA extraction, library preparation, NGS-703 sequencing, and barcode abundance quantification.

TraCe-Seq barcode assignment: TraCe-Seq barcode recovery from scRNA-Seq FASTQs was performed using Salmon (v. 0.1.1). Briefly, custom augmented Salmon index was created using human reference GRCh38 and transgene GFP fused to one of 100,000 30-nt GC-optimized barcode in library. Transcript expression of demultiplexed per-cell FASTQs were then quantified using custom Salmon index. Cells with single GFP-barcode expression were assigned to corresponding TraCe-Seq barcode. Cells with multiple TraCe-Seq barcodes expressed were assigned to a single TraCe-Seq barcode if expression one TraCe-Seq was 3-fold higher than other detected TraCe-Seq barcodes.

TraCe-Seq barcode enrichment/depletion analyses: Non-parametric local regression was applied to TraCe-Seq barcode prevalence in baseline sample compared to each treatment population. Enriched TraCe-Seq barcodes after treatment were those barcodes greater than 6 from local regression. Depleted TraCe-Seq barcodes were determined as those TraCe-Seq barcodes whose log 2 fold-change was greater the mean plus standard deviation of TraCe-Seq population.

Bulk RNA-seq data processing: For RNA-seq data analysis, RNA-seq reads were first aligned to ribosomal RNA sequences to remove ribosomal reads. The remaining reads were aligned to the human reference genome (GRCh38) using GSNAP version “2013-10-10,” allowing a maximum of two mismatches per 75 base sequence (parameters: ‘-M 2 -n 10 -B 2 -i 1-N 1-w 200000 -E 1--pairmax-rna=200000 --clip-overlap). Transcript annotation was based on the Ensembl genes database (release 77). To quantify gene expression levels, the number of reads mapped to the exons of each RefSeq gene was calculated.

Gene set enrichment analysis: KEGG gene signatures enrichment was performed using clusterProfiler (v.3.14.3). Gene set enrichment of KEGG signatures was calculated using nPerm=1000 and minGSSIze=120.

Trajectory inference analysis: Erlotinib-, GNE-104-, and GNE-069-treated cells were subset and re-clustered using Seurat (see “scRNA-Seq data processing and analysis”). Slingshot 1.5.2 was used to order and project cells into principal curves representing distinct trajectories. Default Slingshot parameters were used to identify starting cluster and number/path of trajectories. Length of each trajectory represents pseudotime, a computational parameter denoting progress along a process. For visualizations, pseudotime was normalized across entire length of trajectory to enable comparisons for expression trends.

### REFERENCES

1. M. Yu et al., A resource for cell line authentication, annotation and quality control. Nature 520, 307-311 (2015).
2. C. Galdeano et al., Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J Med Chem 57, 8657-8663 (2014).
3. N. Blaquiere et al., Preparation of (4-hydroxypyrrolodin-2-yl)-hydroxamate compounds. And methods of use thereof as modulators of targeted ubiquitination. WO2019084026, May 2, 2019.
4. R. C. Schnur et al. Preparation of N-phenylquinazoline-4-amines as neoplasm inhibitors. WO9630347, Oct. 3, 1996.
5. N. Blaquiere et al., HETERO-BIFUNCTIONAL DEGRADER COMPOUNDS AND THEIR USE AS MODULATORS OF TARGETED UBIQUINATION (VHL) WO2019183523, Sep. 26, 2019
6. Y. Jia et al., Overcoming EGFR(T790M) and EGFR(C797S) resistance with mutant-selective allosteric inhibitors. Nature 534, 129-132 (2016).
7. G. Yu, L. G. Wang, Y. Han, Q. Y. He, clusterProfiler: an R package for comparing biological themes among gene clusters. OMICS 16, 28 (2012)
8. K. Street et al., Slingshot: cell lineage and pseudotime inference for single-cell transcriptomics. BMC Genomics 19, 477 (2018).
9. P. B. Chapman et al., Improved survival with vemurafenib in melanoma with BRAF V600E mutation. N Engl J Med 364, 2507-2516 (2011).
10. P. Lito, N. Rosen, D. B. Solit, Tumor adaptation and resistance to RAF inhibitors. Nat Med 19, 1401-1409 (2013).
11. Y. L. Choi et al., EML4-ALK mutations in lung cancer that confer resistance to ALK inhibitors. N Engl J Med 363, 1734-1739 (2010).
12. M. E. Gorre et al., Clinical resistance to STI-571 cancer therapy caused by BCR-ABL gene mutation or amplification. Science 293, 876-880 (2001).
13. S. Kobayashi et al., EGFR mutation and resistance of non-small-cell lung cancer to gefitinib. N Engl J Med 352, 786-792 (2005).
14. W. Pao et al., Acquired resistance of lung adenocarcinomas to gefitinib or erlotinib is associated with a second mutation in the EGFR kinase domain. PLoS Med 2, e73 (2005).
15. R. L. Yauch et al., Epithelial versus mesenchymal phenotype determines in vitro sensitivity and predicts clinical activity of erlotinib in lung cancer patients. Clin Cancer Res 11, 8686-8698 (2005).
16. A. H. Davies, H. Beltran, A. Zoubeidi, Cellular plasticity and the neuroendocrine phenotype in prostate cancer. Nat Rev Urol 15, 271-286 (2018).
17. H. Kitai et al., Epithelial-to-Mesenchymal Transition Defines Feedback Activation of Receptor Tyrosine Kinase Signaling Induced by MEK Inhibition in KRAS-Mutant Lung Cancer. Cancer Discov 6, 754-769 (2016).
18. H. Uramoto et al., Epithelial-mesenchymal transition in EGFR-TKI acquired resistant lung adenocarcinoma. Anticancer Res 30, 2513-2517 (2010).
19. G. M. Burslem, C. M. Crews, Proteolysis-Targeting Chimeras as Therapeutics and Tools for Biological Discovery. Cell 181, 102-114 (2020).
20. R. Verma, D. Mohl, R. J. Deshaies, Harnessing the Power of Proteolysis for Targeted Protein Inactivation. Mol Cell 77, 446-460 (2020).
21. C. M. Olson et al., Pharmacological perturbation of CDK9 using selective CDK9 inhibition or degradation. Nat Chem Biol 14, 163-170 (2018).
22. I. You et al., Discovery of an AKT Degrader with Prolonged Inhibition of Downstream Signaling. Cell Chem Biol 27, 66-73 e67 (2020).
23. A. Dixit et al., Perturb-Seq: Dissecting Molecular Circuits with Scalable Single-Cell RNA Profiling of Pooled Genetic Screens. Cell 167, 1853-1866 e1817 (2016).
24. C. Weinreb, A. Rodriguez-Fraticelli, F. D. Camargo, A. M. Klein, Lineage tracing on transcriptional landscapes links state to fate during differentiation. Science 367, (2020).
25. T. J. Lynch et al., Activating mutations in the epidermal growth factor receptor underlying responsiveness of non-small-cell lung cancer to gefitinib. N Engl J Med 350, 2129-2139 (2004).
26. J. G. Paez et al., EGFR mutations in lung cancer: correlation with clinical response to gefitinib therapy. Science 304, 1497-1500 (2004).
27. K. Sankar, S. M. Gadgeel, A. Qin, Molecular therapeutic targets in non-small cell lung cancer. Expert Rev Anticancer Ther, (2020).
28. J. C. Soria et al., Osimertinib in Untreated EGFR-Mutated Advanced Non-Small-Cell Lung Cancer. N Engl J Med 378, 113-125 (2018).
29. A. Leonetti et al., Resistance mechanisms to osimertinib in EGFR-mutated non-small cell lung cancer. Br J Cancer 121, 725-737 (2019).
30. D. P. Bondeson et al., Catalytic in vivo protein knockdown by small-molecule PROTACs. Nat Chem Biol 11, 611-617 (2015).
31. K. N. Shah et al., Aurora kinase A drives the evolution of resistance to third-generation EGFR inhibitors in lung cancer. Nat Med 25, 111-118 (2019).
32. S. V. Sharma et al., A chromatin-mediated reversible drug-tolerant state in cancer cell subpopulations. Cell 141, 69-80 (2010).
33. L. A. Byers et al., An epithelial-mesenchymal transition gene signature predicts resistance to EGFR and PI3K inhibitors and identifies Axl as a therapeutic target for overcoming EGFR inhibitor resistance. Clin Cancer Res 19, 279-290 (2013).
34. N. Okura et al., ONO-7475, a Novel AXL Inhibitor, Suppresses the Adaptive Resistance to Initial EGFR-TKI Treatment in EGFR-Mutated Non-Small Cell Lung Cancer. Clin Cancer Res 26, 2244-2256 (2020).
35. H. Taniguchi et al., AXL confers intrinsic resistance to osimertinib and advances the emergence of tolerant cells. Nat Commun 10, 259 (2019).
36. Z. Zhang et al., Activation of the AXL kinase causes resistance to EGFR-targeted therapy in lung cancer. Nat Genet 44, 852-860 (2012).
37. A. N. Hata et al., Tumor cells can follow distinct evolutionary paths to become resistant to epidermal growth factor receptor inhibition. Nat Med 22, 262-269 (2016).
38. M. C. Wagle et al., A transcriptional MAPK Pathway Activity Score (MPAS) is a clinically relevant biomarker in multiple cancer types. NPJ Precis Oncol 2, 7 (2018).
39. Y. Jia et al., Overcoming EGFR(T790M) and EGFR(C797S) resistance with mutant-selective allosteric inhibitors. Nature 534, 129-132 (2016).
40. C. To et al., Single and Dual Targeting of Mutant EGFR with an Allosteric Inhibitor. Cancer Discov 9, 926-943 (2019).
41. S. J. Marciniak et al., CHOP induces death by promoting protein synthesis and oxidation in the stressed endoplasmic reticulum. Genes Dev 18, 3066-3077 (2004).
42. P. Walter, D. Ron, The unfolded protein response: from stress pathway to homeostatic regulation. Science 334, 1081-1086 (2011).
43. E. Timosenko et al., Nutritional Stress Induced by Tryptophan-Degrading Enzymes Results in ATF4-Dependent Reprogramming of the Amino Acid Transporter Profile in Tumor Cells. Cancer Res 76, 6193-6204 (2016).
44. H. H. Rabouw et al., Small molecule ISRIB suppresses the integrated stress response within a defined window of activation. Proc Natl Acad Sci USA 116, 2097-2102 (2019).
45. D. Buckley, et al. Targeting the von Hippel-Lindau E3 ubiquitin ligase using small molecules to disrupt the VHL/HIF-la interaction. J Am Chem Soc 134, 4465-8 (2012).
46. C. Hetz, K. Zhang, R. J. Kaufman, Mechanisms, regulation and functions of the unfolded protein resonse. Nat Rev Mol Cell Biol 21, 421-438 (2020).
47. S. Stockwell, et al., Mechanism-based screen for G1-S checkpoint acitvators identifies a selective activator of EIFAK3/PERK signaling PLoS One 7, e28568 (2012).
48. J. Bruch, et al., PERK activation mitigates tau pathology in vitro and in vivo. EMBO Mol Med 9, 371-384 (2017).

1. A method for screening cells for a trait, the method comprising:
(a) obtaining a plurality of barcoded cells, wherein each barcoded cell comprises a single, unique barcode;
(b) performing a first sequencing of RNA and/or DNA on a subset of the plurality of barcoded cells;
(c) culturing the plurality of barcoded cells in the presence of a selection pressure for a first period of time, thereby forming a first plurality of cells;
(d) performing a second sequencing of RNA and/or DNA on a subset of the first plurality of cells;
(e) culturing the first plurality of cells in the presence of the selection pressure for a second period of time, thereby forming a second plurality of cells;
(f) performing a third sequencing of RNA and/or DNA on at least a subset of the second plurality of cells; and
(g) determining a level of a barcode sequenced in the first sequencing, second sequencing, and/or third sequencing.

2. A method for screening cells for a response trait to a therapeutic agent, the method comprising:
(a) obtaining a plurality of barcoded cells, wherein each barcoded cell comprises a single, unique barcode;
(b) performing a first sequencing of RNA and/or DNA on a subset of the plurality of barcoded cells;
(c) culturing the plurality of barcoded cells in the presence of the therapeutic agent for a first period of time, thereby forming a first plurality of cells;
(d) performing a second sequencing of RNA and/or DNA on a subset of the first plurality of cells;
(e) culturing the first plurality of cells with the therapeutic agent for a second period of time, thereby forming a second plurality of cells;
(f) performing a third sequencing of RNA and/or DNA on a subset of the second plurality of cells; and
(g) determining a level of a barcode sequenced in the first sequencing, second sequencing, and/or third sequencing.

3.-41. (canceled)

42. A method for comparing responses to selective pressures, the method comprising:
(a) obtaining a first plurality of barcoded cells, wherein each barcoded cell comprises a single, unique barcode;
(b) obtaining a second plurality of barcoded cells that is substantially similar to the first plurality of barcoded cells;
(c) performing a first sequencing of RNA and/or DNA from the first plurality of barcoded cells and/or the second plurality of barcoded cells;
(d) culturing the first plurality of barcoded cells in the presence of a first selection pressure, thereby forming a first plurality of cells;
(e) culturing the second plurality of barcoded cells in the presence of a second selection pressure, thereby forming a second plurality of cells;
(f) performing a second sequencing of RNA and/or DNA from the first plurality of cells and/or the second plurality of cells;
(g) culturing the first plurality of cells in the presence of the first selection pressure, thereby forming a third plurality of cells;
(h) culturing the second plurality of cells in the presence of the second selection pressure, thereby forming a fourth plurality of cells;
(i) performing a third sequencing of RNA and/or DNA from the third plurality of cells and/or the fourth plurality of cells; and
(j) determining a level of one or more barcodes sequenced in the first sequencing, second sequencing, and/or third sequencing
wherein steps (g) to (i) are repeated for one or more iterations, thereby forming one or more subsequent pluralities of cells and one or more subsequent sequencings.

43. (canceled)

44. The method of claim 42, wherein step (j) further comprises determining a level of one or more barcodes in the one or more subsequent sequencing steps.

45. The method of claim 42, wherein the first plurality of barcoded cells and the second plurality of barcoded cells comprise a plurality of clonal populations, wherein each cell within a single clonal population comprises the same single, unique barcode.

46. The method of claim 42, wherein the single, unique barcode is a unique combination of barcodes.

47. The method of claim 45, wherein the relative abundance of cells in each clonal population is approximately equal to the number of cells in each other clonal population in steps (a) and (b).

48. The method of claim 47, wherein from the first sequencing step the relative abundance of cells in a clonal population is determined relative to the number of cells comprising the barcode(s).

49. The method of claim 42, further comprising identifying a barcode(s) that is enriched in the first plurality of cells and/or the second plurality of cells.

50. The method of claim 49, further comprising identifying one or more genes having higher levels of expression in cells comprising the enriched barcode(s).

51. The method of claim 42, further comprising determining a first level of expression of a gene in cells having a barcode(s) enriched in the first plurality of cells and/or third plurality of cells and/or one or more subsequent pluralities of cells based on the second sequencing and/or third sequencing and/or one or more subsequent sequencings, and a second level of expression of the gene in the second plurality of barcoded cells, and/or fourth plurality of cells and/or one or more subsequent pluralities of cells based on the first sequencing, second sequencing, and/or third sequencing and/or one or more subsequent sequencings.

52. The method of claim 51, further comprising comparing the first level of expression of the gene to the second level of expression of the gene.

53. A method of screening cells for a trait in a cell, comprising:
(a) providing a mixture of cells comprising multiple clonal populations wherein each clonal population comprises an identifier that is unique to the respective clonal populations, and wherein initial genetic, transcriptomic, and/or proteomic information of at least one representative member of each clonal population is known;
(b) culturing the mixture of cells in the presence of a first selective pressure for a first period of time, and at the end of the first period of time, obtaining second genetic, transcriptomic, and/or proteomic information for at least one member of a surviving clonal population from within the mixture of cells;
(c) subjecting the mixture of cells that were subjected to the first selective pressure to a second selective pressure for a second period of time, and at the end of the second period of time, obtaining third genetic, transcriptomic, and/or proteomic information of at least one member of a surviving clonal population from within the mixture of cells; and
(d) determining the level of a clonal population present in the final mixture of cells based upon the unique identifier for the clonal population.

54. The method of claim 53, wherein step (b) or (c) is repeated for one or more iterations.

55. (canceled)

56. The method of claim 53, wherein steps (b) and (c) are repeated for one or more iterations.

57. The method of claim 53, further comprising identifying an adaptive trait, wherein the adaptive trait is a genetic and/or proteomic trait present in or absent from a clonal population in the final mixture of cells.

58. The method of claim 57, wherein the adaptive trait is a presence or absence of a gene, allele, genetic modification, transcript, or protein; or a change in a gene, allele, transcript, or protein when comparing the first, second and/or third and/or one or more subsequent genetic, transcriptomic, and/or proteomic information obtained.

59. The method of claim 53, wherein step (b) comprises obtaining fourth genetic, transcriptomic, and/or proteomic information of at least one member of a second surviving clonal population from within the mixture of cells.

60. The method of claim 53, wherein step (c) comprises obtaining fifth genetic, transcriptomic, and/or proteomic information of at least one member of a second surviving clonal population from within the mixture of cells.

61. The method of claim 53, further comprising comparing information from the initial genetic, transcriptomic, and/or proteomic information, second genetic, transcriptomic, and/or proteomic information, and/or third genetic, transcriptomic, and/or proteomic information, and/or one or more subsequent genetic, transcriptomic, and/or proteomic information.

62.-64. (canceled)

65. The method of claim 53, wherein obtaining the genetic, transcriptomic, and/or proteomic information comprises RNA-seq, single cell RNA-seq, DNA sequencing, epigenetic sequencing, or protein sequencing.

66.-71. (canceled)

72. The method of claim 53, wherein the first selective pressure and/or the second selective pressure comprises treatment with a therapeutic agent, contact with a contaminant, genomic engineering, engraftment into a host, a culture condition, a growth condition, contact with a stimulus, or contact with other cells.

73. A method of identifying a cellular program that facilitates adaptation to a pressure, comprising:
(a) transducing cells with a plurality of barcodes such that each cell contains a single, unique barcode;
(b) expanding the cells in culture to create a starting cell pool of clones of cells containing each barcode;
(c) obtaining first genetic, transcriptomic, and/or proteomic information from a first subset of the starting cell pool;
(d) culturing a second subset of the starting cell pool in the presence of a selective pressure to expand the starting cell pool and form an intermediate cell pool;
(e) obtaining second genetic, transcriptomic, and/or proteomic information from a first subset of the intermediate cell pool;
(f) continuing to culture a second subset of the intermediate cell pool in the presence of the selective pressure to expand the intermediate cell pool and form a final cell pool;
(g) obtaining third genetic, transcriptomic, and/or proteomic information from at least a subset of the final cell pool;
(h) quantifying a level of each barcode in the final cell pool, intermediate cell pool, and/or starting cell pool;
(i) assigning cells with barcodes enriched in the final cell pool as winning clones and/or assigning cells with barcodes depleted in the final cell pool as losing clones; and
(j) determining a genetic mutation, transcription program, and/or protein expression associated with at least one winning clone and/or at least one losing clone
wherein approximately equal numbers of clones of cells containing each barcode are used to create the starting cell pool.

74. (canceled)

75. The method of claim 74, wherein the numbers of clones of cells are normalized relative to the numbers of cells comprising each barcode as obtained in step (c).

76. The method of claim 73, wherein steps (d) and (e) are repeated for one or more iterations thereby obtaining one or more additional intermediate cell pools and one or more additional intermediate genetic, transcriptomic, and/or proteomic information.

77. The method of claim 76, wherein step (h) further comprises quantifying a level of each barcode in the one or more additional intermediate cell pools.

78. The method of claim 73, wherein the single, unique barcode is a unique combination of barcodes.

79. The method of claim 73, wherein the selective pressure comprises treatment with a therapeutic agent, contact with a contaminant, genomic engineering, engraftment into a host, a culture condition, a growth condition, contact with a stimulus, or contact with other cells.

80. The method of claim 73, wherein obtaining the genetic, transcriptomic, and/or proteomic information comprises RNA-seq, single cell RNA-seq, DNA sequencing, epigenetic sequencing, or protein sequencing.

81.-86. (canceled)
